(19) United States
(12) Patent Application Publication
(10) Pub. No.: US 2024/0287071 A1
(43) Pub. Date: Aug. 29, 2024

(54) CLASS OF IMIDAZOLIDINOPYRIMIDONE COMPOUNDS AND USE THEREOF IN TREATMENT OF HSCLPP-MEDIATED DISEASES

(71) Applicant: SICHUAN UNIVERSITY, Chengdu, Sichuan (CN)

(72) Inventors: Youfu LUO, Chengdu, Sichuan (CN); Tao YANG, Chengdu, Sichuan (CN)

(21) Appl. No.: 18/568,523

(22) PCT Filed: Apr. 6, 2022
(86) PCT No.: PCT/CN2022/085402

§ 371 Date: Dec. 8, 2023

(30) Foreign Application Priority Data
Jun. 8, 2021 (CN) 202110640077.5

(51) Int. Cl. C07D 471/14; A61K 31/519; A61P 35/00

(52) U.S. Cl. CPC C07D 471/14; A61K 31/519; A61P 35/00

(57) ABSTRACT

Imidazolidinopyrimidone compounds, or a pharmaceutically acceptable salt, a hydrate, or a crystal form thereof can be used in the treatment of human caseinolytic protease P(HsClpP) mediated diseases. The compounds have a significant activity in regulating and controlling HsClpP and can be used for treating related diseases mediated by HsClpP.

### TECHNICAL FIELD

The present invention relates to a class of imidazolidinopyrimidone compounds, or pharmaceutically acceptable salts, hydrates, or crystal forms thereof in the treatment of HsClpP-mediated diseases, and belongs to the field of chemical medicine.

### BACKGROUND

HsClpP is an ATP-dependent unfoldase peptidase protein complex that exists in mitochondrial matrix. HsClpP maintains dynamic balance of organelles, controls protein quality, regulates mitochondria metabolism, and plays an important role in mitochondrial unfolded protein response and oxidative phosphorylation integrity. Studies have shown that HsClpP is upregulated in various types of cancer. Compared to normal hematopoietic cells from healthy individuals, the expression of HsClpP is increased in 45% of primary acute myeloid leukemia (AML) samples.

Immunohistochemical analysis has shown overexpression of HsClpP in solid tumors such as bladder, prostate, uterus, liver, colon, thyroid, lung, breast, ovary, testis, stomach, lymph nodes, and the central nervous system. By comparing the expression of the HsClpP encoding gene in cancer tissue samples and normal tissue samples from TCGA data, it can be observed that HsClpP is highly expressed in almost all cancer cells. The role of HsClpP in maintaining mitochondrial function is reflected in the timely and accurate degradation of misfolded proteins. When HsClpP is overly activated, this precise degradation pathway coonverts to disorder and non-selective degradation, leading to incorrect degradation of functional proteins, reduced level of respiratory chain proteins and damage of oxidative phosphorylation within the mitochondria. Conversely, the inhibition of HsClpP activity leads to the accumulation of misfolded proteins, damaged proteins, and short lifespan proteins, which destroys oxidative phosphorylation and causes tumor cell impair, or even death. Therefore, both inhibition and activation of HsClpP function can interfere with its normal functioning, resulting in tumor cell damage and even death. Targeting HsClpP for tumor treatment provides a new strategy for the treatment of human mitochondrial-related diseases and the screening and optimization of small molecules.

Some small molecules have been reported to regulate HsClpP protein, including HsClpP inhibitors, such as β-lactones, phenyl esters, and boronic acid peptidomimetics. HsClpP activators include ADEP compounds, D9, and imipridone compounds. The structure of imipridone compounds is characterized by a core structure of imidazolino dihydropyrimidinone, wherein the imidazolineis connected to other substituents through the nitrogen ring atom of the imidazoline. Among them, ONC201 and ONC206 have been approved for clinical trials in tumor treatment. Clinical trials of ONC201 targeting multiple tumors are currently entering Phase II, and clinical trials targeting H3K27M mutant gliomas have entered Phase III. ONC206 was approved in 2020 for Phase I clinical research on recurrent central nervous system tumors.

### SUMMARY

The purpose of the present invention is to provide a class of imidazolidinopyrimidonef compounds. Another purpose of the present invention is to provide a use of the compound. Specifically, the first aspect of the present invention provides a compound as represented by Formula I, or a pharmaceutically acceptable salt, a hydrate, or a crystalline form thereof:

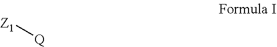

wherein Z₁ is independently selected from H, alkyl, cycloalkyl, cycloalkylalkyl, heterocycloalkyl, heterocycloalkylalkyl, aryl, heteroaryl, arylalkyl, heteroarylalkyl, alkoxyalkyl, alkoxycarbonyl, arylalkoxy(aryloxy), arylalkylthio(arylthio), and acyl radical, Q is independently selected from the group consisting of:

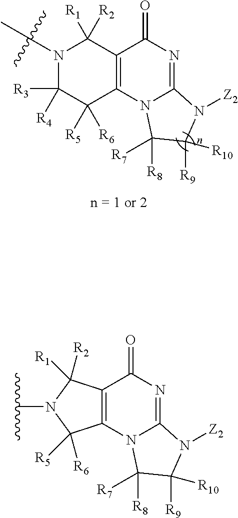

wherein, in each formula, R₁ to R₆ are each independently selected from hydrogen, halogen, C3-C6 cycloalkyl, C1-C6 substituted or unsubstituted alkyl; each of R7 to R10 is independently selected from hydrogen, halogen, C3-C6 cycloalkyl, C1-C6 substituted or unsubstituted alkyl; Z₂ is independently selected from alkyl, cycloalkyl, cycloalkylalkyl, heterocycloalkyl, heterocycloalkylalkyl, aryl, heteroaryl, arylalkyl, heteroarylalkyl, alkoxyalkyl, alkoxycarbonyl, arylalkoxy, arylalkylthio, and acyl; and n=1 or 2.

In another preferred embodiment, R₁ to R₆ are each independently selected from hydrogen, halogen, substituted or unsubstituted C1-C3 alkyl.

In another preferred embodiment, the substituted or unsubstituted C1-C6 alkyl is C1-C6 haloalkyl, preferably C1-C3 haloalkyl.

In another preferred embodiment, the compound is represented by Formula I-1:

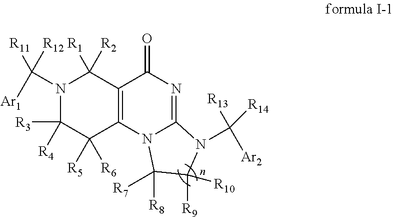

wherein, Ar₁ and Ar₂ are independently selected from aryl, heteroaryl, alkyl, alkenyl, alkynyl, arylalkyl, cycloalkyl, cycloalkylalkyl, and heterocycloalkyl; the aryl, heteroaryl, alkyl, alkenyl, alkynyl, arylalkyl, cycloalkyl, cycloalkylalkyl, or heterocycloalkyl independently have 0-5 (such as 1, 2, 3, or 4) R₁₅ substituents; each R₁₅ is independently selected from halogen, cyano, C1-C6 alkyl, C3-C9 substituted or unsubstituted cycloalkyl, C1-C6 haloalkyl, —CF₃, —NH₂, —NO₂, —SH, —SR₁₆, —OH, C1-C6 substituted or unsubstituted alkoxy, —NR₁₆R₁₇, (C3-C9) cycloalkyl, (C2-C6) alkynyl, (C4-C8) cycloalkenyl, (C4-C8) cycloalkenylalkyl, substituted or unsubstituted aryl, substituted or unsubstituted heteroaryl, -COOH, —COOR₁₆, —OCOOR₁₆, (C2-C8) alkenyl, —SO₂OR₁₆, —SO₂NR₁₆R₁₇, —SO₂R₁₆, —NR₁₆SO₂R₁₇, —CONR₁₆R₁₇, —COR₁₆, —NR₁₆COR₁₇; R₁ to R₆, R₇ to R₁₀ are each independently selected from hydrogen, halogen, C3-C6 cycloalkyl, C1-C6 substituted or unsubstituted alkyl; each of R₁₁ to R₁₇ is independently selected from hydrogen, halogen, C1-C3 substituted or unsubstituted alkyl, or R₁₁ and R₁₂ together with the connected C atom form a carbonyl group (C═O); and n=1 or 2.

Furthermore, the compound is represented by Formula I-1, wherein Ar₁ and Ar₂ are independently selected from aryl, heteroaryl; the aryl, and heteroaryl contain 0-5 R₁₁ substituents, where R₁₁ is selected from halogen, cyano, C1-C6 alkyl, C3-C9 substituted or unsubstituted cycloalkyl, C1-C6 haloalkyl, —CF₃, —NH₂, —NO₂, —SH, —SR₁₁, —OH, C1-C6 substituted or unsubstituted alkoxy, —NR₁₂R₁₃, (C3-C9) cycloalkyl, (C2-C6) alkynyl, (C4-C8) cycloalkenyl, (C4-C8) cycloalkenylalkyl, substituted or unsubstituted aryl, substituted or unsubstituted heteroaryl, -COOH, —COOR₁₆, —OCOOR₁₆, (C2-C6) alkynyl, (C2-C8) alkenyl, —SO₂OR₁₆, —SO₂NR₁₆R₁₇, —SO₂R₁₆, —NR₁₆SO₂R₁₇, —CONR₁₆R₁₇, —COR₁₆, —NR₁₆COR₁₇; R₁ to R₆, R₇ to R₁₀ are each independently selected from hydrogen, halogen, C3-C6 cycloalkyl, C1-C6 substituted or unsubstituted alkyl; R₁₁ to R₁₇ are each independently selected from hydrogen, halogen, C1-C3 substituted or unsubstituted alkyl.

In another preferred embodiment, Ar₁ and Ar₂ are independently selected from the group consisting of: phenyl, naphthyl, quinolinyl, indolyl, benzofuranyl, pyridyl, thiadiazolyl, thiazolyl, C1-C6 alkyl, C2-C6 alkenyl, C2-C6 alkynyl, C3-C8 cycloalkyl, C3-C8 cycloalkyl-C1-C3 alkylene, phenyl-C1-C3 alkylene, thienyl, and furanyl; preferably, the phenyl, naphthyl, quinolinyl, indolyl, benzofuranyl, pyridyl, thiadiazolyl, thiazolyl, C1-C6 alkyl, C2-C6 alkenyl, C2-C6 alkynyl, C3-C8 cycloalkyl, C3-C8 cycloalkyl-C1-C3 alkylene, phenyl-C1-C3 alkylene, thienyl, and furanyl are independently and optionally substituted with 0-5 R₁₅ substituents, and each R₁₅ is independently selected from the group consisting of: halogen, —OH, cyano, C1-C6 alkyl, C1-C6 alkoxy, C1-C6 haloalkyl, —SO₂C1-C3 alkyl, COOC1-C6 alkyl, COOH.

In another preferred embodiment, Ar₁ is selected from the group consisting of: phenyl, phenyl-C1-C3 alkylene, pyridyl, thiadiazolyl, C1-C6 alkyl, C2-C6 alkenyl, C2-C6 alkynyl, C3-C8 cycloalkyl, C3-C8 cycloalkyl-C1-C3 alkylene, phenyl-C1-C3 alkylene, thienyl, and furanyl; preferably, the phenyl, pyridyl, C1-C6 alkyl, C2-C6 alkenyl, C2-C6 alkynyl, C3-C8 cycloalkyl, C3-C8 cycloalkyl-C1-C3 alkylene, phenyl-C1-C3 alkylene, thienyl, and furanyl are each independently substituted with 0-5 R₁₅ substituents, and each R₁₅ is independently selected from the group consisting of: halogen, —OH, cyano, C1-C6 alkyl, C1-C6 alkoxy, C1-C6 haloalkyl.

In another preferred embodiment, Ar₂ is selected from the group consisting of: phenyl, pyridyl, C1-C6 alkyl, C2-C6 alkenyl, C2-C6 alkynyl, phenyl-C1-C3 alkylene, C3-C8 cycloalkyl, and thienyl; wherein the phenyl, pyridyl, C1-C6 alkyl, C2-C6 alkenyl, C2-C6 alkynyl, phenyl-C1-C3 alkylene, C3-C8 cycloalkyl, or thienyl are optionally and independently substituted with 0-5 R₁₅ substituents, and each R₁₅ is independently selected from the group consisting of: halogen, —OH, cyano, C1-C6 alkyl, C1-C6 alkoxy, C1-C6 haloalkyl, —SO₂C1-C3 alkyl, COOC1-C6 alkyl, COOH.

In another preferred embodiment, Ar₁ is selected from the group consisting of: phenyl, 4-cyanophenyl, 3-cyanophenyl, 2-cyanophenyl, 3-fluorophenyl, 4-fluorophenyl, 2-fluorophenyl, 3, 4-difluorophenyl, 2, 4-difluorophenyl, pyridine-2-yl, pyridine-3-yl, pyridine-4-yl, 2-methylphenyl, 3-methylphenyl, 4-methylphenyl, 2-methoxyphenyl, 3-methoxyphenyl, 4-methoxyphenyl, cyclopropyl, cyclobutyl, cyclopentyl, cyclohexyl, furan-3-yl, ethynyl, and C1-C6 alkyl.

In another preferred embodiment, Ar₂ is selected from the group consisting of: phenyl, 2-methoxyphenyl, 3-methoxyphenyl, 4-methoxyphenyl, 2-methylphenyl, 3-methylphenyl, 4-methylphenyl, 2-trifluoromethylphenyl, 3-trifluoromethylphenyl, 4-trifluoromethylphenyl, 2-fluorophenyl, 2-chlorophenyl, 2-bromophenyl, 3-fluorophenyl, 3-chlorophenyl, 3-bromophenyl, 4-fluorophenyl, 4-chlorophenyl, 4-bromophenyl, 3, 4-difluorophenyl, 3, 4-dichlorophenyl, 3, 4-dibromophenyl, 2, 4-difluorophenyl, 2, 4-dichlorophenyl, 2, 4-dibromophenyl, 2-bromo-3-fluorophenyl, 3-bromo-4-fluorophenyl, 2-chloro-4-fluorophenyl, 4-CH₃SO₂-phenyl, cyclopropyl, cyclobutyl, cyclopentyl, ethynyl, cyclohexyl, C1-C6 alkyl, phenyl-methylene, pyridin-2-yl, pyridin-3-yl, pyridin-4-yl, and 3-CH₃COO-phenyl.

In another preferred embodiment, R₁, R₂, R₃, R₄, R₅, R₆ are independently selected from hydrogen, halogen, C1-C3 halogenated alkyl.

In another preferred embodiment, R₇, R₈, R₉, R₁₀ are independently selected from hydrogen, halogen, C1-C3 halogenated alkyl.

In another preferred embodiment, R₁₁, R₁₂, R₁₃, R₁₄ are independently selected from hydrogen, halogen, C1-C3 halogenated alkyl.

In another preferred embodiment, R₁₁ and R₁₂ together with the attached C atom form a carbonyl group (C═O).

In another preferred embodiment, R₅ and R₆ are independently halogen, preferably, R₅ and R₆ are F.

In another preferred embodiment, R₁-R₁₄ are all H.

In another preferred embodiment, R₁-R₄ and R₇-R₁₄ are all H, and R₅ and R₆ are independently halogen.

In another preferred embodiment, R₁-R₁₄, Ar₁, and Ar₂ are each independently the corresponding groups in any one of compounds 1-81.

In another preferred embodiment, n=1.

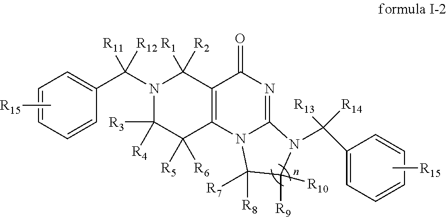

Further preferred structure is represented by Formula I-2, wherein Ar₁ and Ar₂ are independently selected from phenyl, and the phenyl each independently have 0-5 R₁₅ substituents; each R₁₅ is independently selected from halogen, cyano, C1-C6 alkyl, C3-C9 substituted or unsubstituted cycloalkyl, C1-C6 haloalkyl, —CF₃, —NH₂, —NO₂, —SH, —SR₁₁, —OH, C1-C6 substituted or unsubstituted alkoxy, —NR₁₂R₁₃, (C3-C9) cycloalkyl, (C2-C6) alkynyl, (C4-C8) cycloalkenyl, (C4-C8) cycloalkenylalkyl, substituted or unsubstituted aryl, substituted or unsubstituted heteroaryl, -COOH, —COOR₁₆, —OCOOR₁₆, (C2-C8) alkenyl, —SO₂OR₁₆, —SO₂NR₁₆R₁₇, —SO₂R₁₆, —NR₁₆SO₂R₁₇, —CONR₁₆R₁₇, —COR₁₆, —NR₁₆COR₁₇; R₁ to R₆, R₇ to R₁₀ are each independently selected from hydrogen, halogen, C3-C6 cycloalkyl, C1-C6 substituted or unsubstituted alkyl; R₁₁ to R₁₄, R₁₆ to R₁₇ are each independently selected from hydrogen, halogen, C1-C3 substituted or unsubstituted alkyl.

In another preferred embodiment, Ar₂ is phenyl and has 1, 2, 3, 4, or 5 R₁₅ substituents, wherein each R₁₅ is independently selected from halogen, C1-C6 alkyl, and C1-C6 haloalkyl; preferably, Ar₂ has 2 R₁₅ substituents.

In another preferred embodiment, Ar₁ is phenyl and has 1, 2, 3, 4, or 5 R₁₅ substituents, wherein each R₁₅ is independently selected from halogen, CN, C1-C6 alkyl, and C1-C6 haloalkyl; preferably, Ar₁ has 1 R₁₅ substituent.

Preferably, Ar₁ and Ar₂ are independently selected from phenyl with 0-5 R₁₅ substituents; each R₁₅ is independently selected from hydrogen, halogen, cyano, —CH₃, —CF₃; R₁-R₁₄ are independently selected from hydrogen, halogen, C1-C3 substituted or unsubstituted alkyl.

In another preferred embodiment, the compound is selected from:

[chemical structure]

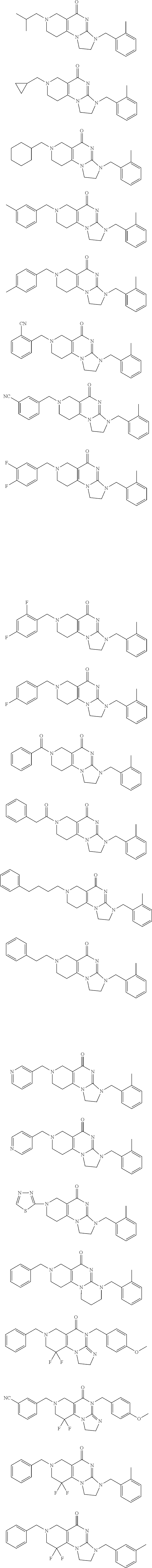

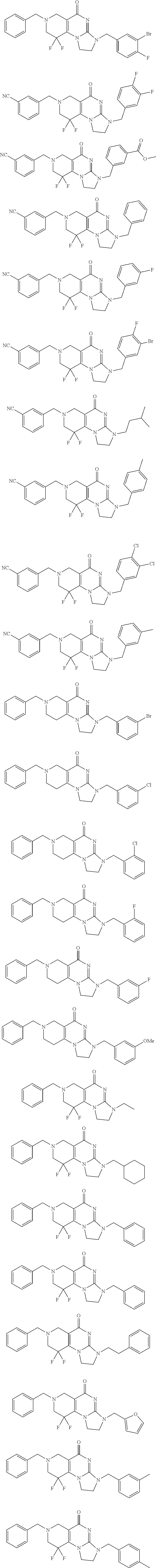

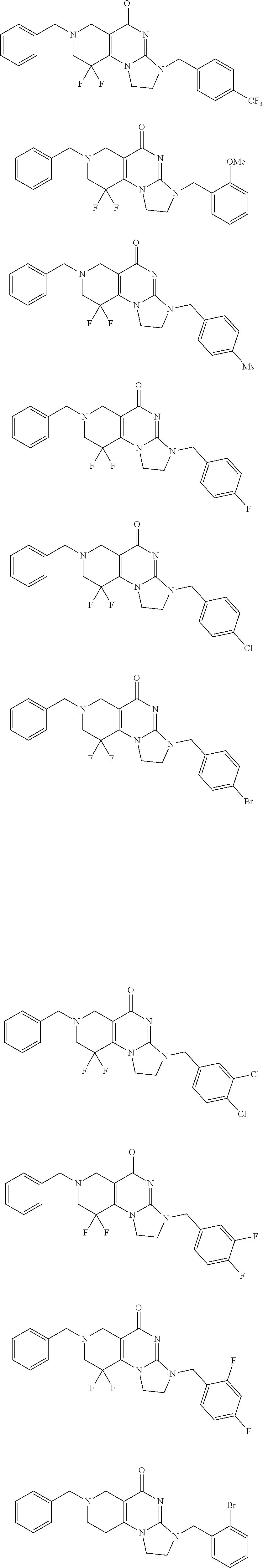

[chemical structure]

In another preferred embodiment, any one or more atoms of the compound are substituted with isotopes, preferably deuterium.

In another preferred embodiment, the compound further includes pharmaceutically acceptable salts, hydrates, solvates, or crystal forms of the compound, preferably, the pharmaceutically acceptable salts are the pharmaceutically acceptable salts formed by the compound in combination with hydrochloric acid, hydrobromic acid, hydrofluoric acid, sulfuric acid, phosphoric acid, nitric acid, formic acid, acetic acid, propionic acid, oxalic acid, malonic acid, succinic acid, fumaric acid, maleic acid, lactic acid, malic acid, tartaric acid, citric acid, picric acid methanesulfonic acid, ethanesulfonic acid, hydroxyethanesulfonic acid, p-toluenesulfonic acid, benzenesulfonic acid, naphthalenesulfonic acid, trifluoroacetic acid, or aspartic acid.

In a second aspect of the invention, provided is a pharmaceutical composition, comprising the compound according to the first aspect of the present invention, or a pharmaceutically acceptable salt, a hydrate, a solvate, or a crystal form thereof as active ingredients, and pharmaceutically acceptable excipients.

A pharmaceutical composition for treating HsClpP-mediated diseases is a preparation prepared by the compound according to the first aspect of the present invention, or a pharmaceutically acceptable salt, a hydrate, a solvate, or a crystal form thereof as active ingredients, and pharmaceutically acceptable excipients or/and auxiliary components.

In a third aspect of the invention, provided is a use of the compound according to the first aspect of the present invention, or a pharmaceutically acceptable salt, a hydrate, a solvate, or a crystal form thereof, or the pharmaceutical compositions containing it in the preparation of drugs for the prevention and/or treatment of HsClpP-mediated neurological diseases, metabolic syndrome and tumor-related diseases.

In a fourth aspect of the invention, provided is a use of the compound according to the first aspect of the present invention, or a pharmaceutically acceptable salt, a hydrate, a solvate, or a crystal form thereof, or the pharmaceutical compositions containing it in the preparation of drugs for the prevention and/or treatment of tumors.

Preferably, the tumors are selected from the group consisting of central nervous system tumors, brain tumors, peripheral nervous system tumors, chromaffin cell tumors, paragangliomas, neuroendocrine tumors, liver cancer, lung cancer, gastric cancer, colon cancer, rectal cancer, pancreatic cancer, breast cancer, prostate cancer, endometrial cancer, hematological malignancies, lymphocytic malignancy, glioblastomas, myeloid mononuclear leukemia, Burkitt's lymphoma, non-small cell lung cancer, glioma, colorectal cancer, melanoma, ovarian cancer, or combinations thereof.

In a fifth aspect of the invention, provided is a method for preventing and/or treating neurological disease, metabolic syndrome and tumor-related diseases mediated by HsClpP, comprising administering according to the first aspect of the present invention, or a pharmaceutically acceptable salt, a hydrate, a solvate, or a crystal form thereof, or the pharmaceutical compositions containing it to a subject in need thereof so as to treat the diseases.

In another preferred embodiment, the subject is a mammal, such as a human, rat, or mouse.

According to the above content of the present invention, various other forms of modifications, replacements, or changes can be made based on common technical knowledge and means in this field, without departing from the basic technical ideas of the present invention.

The above aspects of the present invention are described in further detail below in the form of specific embodiments. However, this should not be construed to mean that the orientation of the above-mentioned subject matter of the present invention is limited to the following embodiments. Any technology realised on the basis of the above contents of the present invention falls within the scope of the present invention.

### BRIEF DESCRIPTION OF THE DRAWINGS

FIG. 1 shows the Heteronuclear Multiple-Bond Correlation (HMBC) spectrum of Compound 1 of the present invention.

FIG. 2 shows the regulatory effects of Compound 1 and Compound 21 on HsClpP.

FIG. 3 displays the thermodynamic information of the interaction between Compound 1 and Compound 21 with HsClpP.

FIG. 4 demonstrates the impact of Compound 21 at a concentration of 100 uM on the thermodynamic stability of HsClpP.

FIG. 5 depicts the influence of the preferred Compound 21 at a concentration of 100 uM on the thermodynamic stability of HsClpP in HCT116 cells.

FIG. 6 illustrates the effects of the preferred Compound 21 at different concentrations on the mitochondrial membrane potential in HCT116 cells.

FIG. 7 shows the impact of the preferred Compound 21 at various concentrations on the levels of SDHB and ATF4 in HCT116 cells.

FIG. 8 indicates the influence of the preferred Compound 21 at different concentrations on the ROS content in HCT116 cells.

FIG. 9 demonstrates the induction of cell cycle retardant by the preferred Compound 21 at different concentrations in HCT116 cells.

FIG. 10 illustrates the induction of apoptosis in HCT116 cells by the preferred Compound 21 at different concentrations.

FIG. 11 displays the inhibition of the preferred Compound 21 at different concentrations on the clone of human colorectal cancer cell HCT116 .

FIG. 12 shows the inhibition of the preferred Compound 21 at different concentrations on the migration of human colorectal cancer cell HCT116.

FIG. 13 illustrates the inhibitory activity of the preferred Compound 1 and Compound 21 at different concentrations on the proliferation of human normal embryonic kidney cells (HEK293) and rat myocardial cells (H9C2).

FIG. 14 depicts the change in mouse body weight within 14 days after a single administration of the hydrochloride salt of Compound 21(compound 21·2HCl) at a dose of 100 mg/kg.

FIG. 15 shows the blood biochemical indicators of mice 14 days after a single administration of the hydrochloride salt of Compound 21(compound 21·2HCl) at a dose of 100 mg/kg.

FIG. 16 indicates the absence of significant pathological damage in mice 14 days after a single administration of the hydrochloride salt of Compound 21(compound 21·2HCl) at a dose of 100 mg/kg.

FIG. 17 shows the in vivo anti-tumor effects of oral administration of compound 21·2HCl and ONC201·2HCl. BALB/c nude mice with HCT116 CRC xenograft tumors were treated with ONC201·2HCl (100 mg/kg, p.o., twice a week) or 21·2HCl (5 and 10 mg/kg, p.o., twice a week) for 19 days; (A) Observation of tumor size in mice after treatment; (B) Photographs of excised tumor tissue after treatment; (C) Relative change in mouse body weight during treatment; (D) Relative volume change of tumors during treatment. Measurements were taken every other day; N=6 animals per group.

### DETAILED DESCRIPTION OF THE INVENTION

After extensive and in-depth research, the inventors, through extensive screening and testing, have provided a novel class of HsClpP agonist compounds with excellent activity. The compounds of the present invention, compared to existing clinical-stage anti-tumor compound ONC201, exhibit higher HsClpP agonist activity and a better therapeutic window, offering a better choice for the clinical treatment of HsClpP-mediated diseases. The present invention is completed based on this foundation.

### Terms

Unless otherwise defined, all technical and scientific terms used in this article have the same meanings as those commonly understood by those skilled in the art to which the present invention belongs.

As used herein, the terms “consisting of” or “including (comprising)” can be open, semi-closed, and closed. In other words, the terms also include “essentially composed of” or “composed of”.

When describing a substituent through a conventional chemical formula written from left to right, the substituent also includes chemically equivalent substituents obtained by writing the structural formula from right to left. For example, —CH₂O— is equivalent to —OCH₂—.

Unless otherwise stated, the term “alkyl” itself or as part of another substituent refers to straight or branched hydrocarbon groups, and the alkyl may have a specified number of carbon atoms, for example, C1-C6 represents 1-6 carbons, including alkyls with 1, 2, 3, 4, 5, and 6 carbons, or C1-C3 represents 1-3 carbons. Examples of alkyl groups include methyl, ethyl, n-propyl, isopropyl, n-butyl, t-butyl, isobutyl, sec-butyl, n-pentyl, n-hexyl, n-heptyl, n-octyl, and the like.

Unless otherwise stated, the term “alkenyl” refers to unsaturated hydrocarbon groups with one or more double bonds, for example, C2-C8 alkenyl represents alkenyl with 2-8 carbons, such as alkenyls with 3, 4, 5, or 6 carbons. Similarly, the term “alkynyl” refers to unsaturated hydrocarbon groups with one or more triple bonds, for example, C2-C6 alkynyl represents alkynyl with 2-6 carbons, such as alkynyls with 3, 4, or 5 carbons. Examples of such unsaturated hydrocarbon groups include ethenyl, 2-propenyl, crotyl, 2-isopropenyl, 2-(butadienyl), 2, 4-pentadienyl, 3-(1, 4-pentadienyl), ethynyl, 1-and 3-propynyl, 3-butynyl, and higher homologs and isomers.

Unless otherwise stated, the term “cycloalkyl” refers to completely saturated cyclic hydrocarbon rings, and cycloalkyl may have a specified number of ring carbon atoms, for example, C3-C9 cycloalkyl represents cycloalkyls with 3-9 ring carbon atoms, including cycloalkyls with 3, 4, 5, 6, 7, and 8 carbon atoms, such as cyclopropyl, cyclobutyl, cyclopentyl, or cyclohexyl, and “cycloalkyl” also refers to bicyclic and polycyclic hydrocarbon rings, such as bicyclo[2.2.1]heptyl, bicyclo[2.2.2]octyl, and the like. Similarly, “cycloalkenyl” refers tocycloalkenyl having one or two double bonds between ring vertices. For example, C4-C8 cycloalkenyl refers to cycloalkenyls with 4-8 ring carbons, such as cycloalkenyl with 5 or 6 carbons.

Unless otherwise stated, the term “heterocycloalkyl” refers to cyclic hydrocarbon rings containing 1 to 5 (preferably 1, 2, and 3) heteroatoms selected from N, O, and S, where nitrogen and sulfur atoms may optionally be oxidized, and nitrogen atoms may optionally be quaternized. Heterocycloalkyls can be mono-cyclic, bicyclic, or polycyclic systems. Non-limiting examples of heterocycloalkyls include pyrrolidine, imidazolidine, pyrazolidine, butyrolactam, valerolactam, imidazolidinone, hydantoin, dioxolame, phthalimide, piperidine, 1, 4-dioxane, morpholine, thiomorpholine, thiomorpholine-S oxide, thiomorpholine-S, S-oxide, piperazine, pyran, pyridinone, 3-pyrroline, thiapyran, pyranone, tetrahydrofuran, tetrahydrothiophene, quinine ring, and the like. Heterocycloalkyls may be connected to the rest of the molecule through ring carbons or heteroatoms. For terms such as cycloalkyl and heterocycloalkyl, they refer to cycloalkyl or heterocycloalkyl connected to the rest of the molecule through alkyl or alkylene linkers. For example, cyclobutyl methyl(ene)-is a cyclobutyl ring attached to the methylene linker of the remaining part of the molecule.

Unless otherwise stated, the term “alkylene” itself or as part of another substituent refers to a divalent group derived from an alkane, which typically has 1-6 carbon atoms, such as C1-C3 alkylene, such as —CH₂CH₂CH₂CH₂—, —CH₂CH₂CH₂—, —CH₂CH₂—, and —CH₂—. Similarly, “alkenylene” or “alkynylene” refer to unsaturated forms of “alkylene” with double or triple bonds, respectively.

Unless otherwise stated, the terms “alkoxy” or “alkyloxy”, “alkylamine group” or “alkylamine”, in its conventional sense, refers to those alkyl groups that are connected to the rest of the molecule via oxygen atoms or nitrogen atoms, respectively. In addition, the alkylamine group can be monosubstituted or bisubstituted. For example, methylamine, ethylamine, propylamine, isopropylamine, butylamine, isobutylamine, tert butylamine, dimethylamine, diethylamine, dipropylamine, diisopropylamine, dibutylamine, diisobutylamine, di tert butylamine, etc. When bisubstituted, the alkyl portion can be the same or different, and can also be combined with nitrogen atoms connecting to each alkyl to form 3-7-membered rings. Therefore, the groups shown in —NR^{a}R^{b} include pyridine, pyrrolidine, morpholine, azetidinyl, etc.

Unless otherwise stated, the terms “halogenated(halo)” or “halogen” itself or as part of another substituent refer to fluorine, chlorine, bromine, or iodine atoms. In addition, terms such as “haloalkyl” refers to either monohaloalkyl or polyhaloalkyl groups. For example, the term “C1-C3 haloalkyl” includes trifluoromethyl, 2, 2, 2-trifluoroethyl, 4-chlorobutyl, 3-bromopropyl, etc. Unless otherwise stated, the term “aryl” refers to a polyunsaturated (usually aromatic) hydrocarbon group, which can be a mono-cyclic ring, or fused, or covalently connected poly-cyclic ring (three rings at most). The term “heteroaryl” refers to an aryl group (or ring) containing 1 to 5 (preferably 1, 2, and 3) heteroatoms selected from N, O, and S, where nitrogen and sulfur atoms are optionally oxidized and nitrogen atoms are optionally quaternized. Heteroaromatic groups can be connected to the rest of the molecule through heteroatoms. Non limiting examples of aryl groups include phenyl, naphthyl, and biphenyl groups, while non limiting examples of heteroaryl groups include pyridinyl, pyridazinyl, pyrazinyl, pyrimidinyl, triazinyl, quinolyl, quinoxalinyl, quinazolinyl, cinnolinyl, phthalazinyl, benzotriazinyl, purinyl, benzimidazolyl, benzopyrazolyl, benzotriazolyl, benzoisoxazolyl, isobenzofuryl, isoindolyl, indolizinyl, benzotriazinyl, thienopyridinyl, thienopyrimidinyl, pyrazolopyrimidinyl, imidazopyridinyl, benzothiazolyl, benzofuranyl, benzothiophenyl, indolyl, quinolinyl, isoquinolinyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furanyl, thienyl, and so on. The substituents of the aromatic and heteroaromatic ring systems mentioned above are selected from the following acceptable groups of substituents.

For the sake of simplicity, when the term “aryl” is used in combination with other terms (such as aryloxy, aryl thio, aryl alkyl-S-(aryl alkyl-S-), aryl alkoxy (aryl alkyl-O—), aralkyl), it includes aryl and heteroaryl rings as defined above. Therefore, the term “aralkyl” or “heteroaralkyl” refers to those functional groups (such as benzyl, phenylethyl, pyridinyl methyl, etc.) that the aryl or heteroaryl groups connected to the alkyl groups that attached to the rest of the molecule. Unless otherwise stated, the term “alkoxycarbonyl” refers to alkyl-O—C(═O)-—, such as C1-C6 alkyl-O—C(═O)—, more specifically CH₃-O—C(═O)—, CH₃CH₂-O—C(═O)—, etc.

Unless otherwise stated, the term “acyl” refers to alkyl-C(═O)—, such as C1-C6 alkyl-C(═O)—, more specifically CH₃—C(═O)—, CH₃CH₂—C(═O)—, etc.

In some embodiments, the above terms (such as “alkyl, ” “cycloalkyl, ” “heterocycloalkyl, ” “aryl, ” and “heteroaryl, ” etc.) will include substituted forms of designated functional groups and unsubstituted forms. Unless otherwise specified, each functional group independently and optionally has one or more substituents (such as 0, 1, 2, 3, 4, or 5) selected from the group consisting of: halogen, cyano, C1-C6 alkyl, C3-C9 substituted or unsubstituted cycloalkyl, C1-C6 haloalkyl, —CF₃, —NH₂, —NO₂, —SH, —SR₁₆, —OH, C1-C6 substituted or unsubstituted alkoxy, —NR₁₆R₁₇, (C3-C9) cycloalkyl, (C2-C6) alkynyl, (C4-C8) cycloalkenyl, (C4-C8) cycloalkenyl alkyl, substituted or unsubstituted aryl, substituted or unsubstituted heteroaryl, —COOH, —COOR₁₆, —OCOOR₁₆, C2-C8 alkenyl, —SO₂OR₁₆, —SO₂NR₁₆R₁₇, —SO₂R₁₆, —NR₁₅SO₂R₁₆, —CONR₁₆R₁₇, —COR₁₆, —NR₁₆COR₁₇; each R₁₆ and R₁₇ is independently selected from hydrogen, halogen, C1-C3 alkyl or C1-C3 halogenated alkyl groups.

Unless otherwise stated, the term “substituted” refers to one or more (e.g., 1, 2, 3, 4, or 5) hydrogen atoms on a functional group being independently replaced by substituents independently selected from the group consisting of hydrogen, halogen, CN, C1-C6 alkyl, C2-C6 alkenyl, C2-C6 alkynyl, C1-C6 haloalkyl, C1-C6 alkoxy, C3-C6 cycloalkyl, aryl, and benzyl.

### Active Ingredient

The present invention provides a compound of formula I,

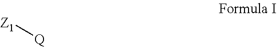

wherein, Z₁, Q are as defined above. Preferably, the compound is represented by formula I-1:

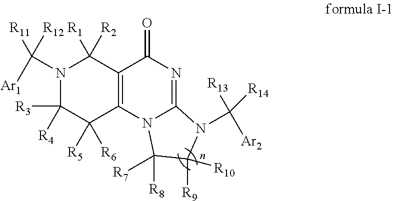

wherein, n, R₁, R₂, R₃, R₄, R₅, R₆, R₇, R₈, R₉, R₁₀, R₁₁, R₁₂, R₁₃, R₁₄, Ar₁ and Ar₂ are as defined above.

Furthermore, when both Ar₁ and Ar₂ are substituted or unsubstituted phenyl, the compound is represented by formula I-2:

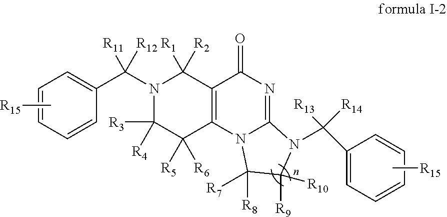

Wherein, R₁, R₂, R₃, R₄, R₅, R₆, R₇, R₈, R₉, R₁₀, R₁₁, R₁₂, R₁₃, R₁₄, R₁₅ are as defined above, wherein, each R₁₅ is independently selected from 0 to 5.

Additionally, the active ingredient of the present invention further includes pharmaceutically acceptable salts, hydrates, solvates, crystal forms, isotope-labeled compounds thereof, or combinations thereof.

Typically, the pharmaceutically acceptable salts may include (but are not limited to): hydrochloride, hydrobromide, hydrofluoride, sulfate, phosphate, nitrate, formate, acetate, propionate, oxalate, malonate, succinate, fumarate, maleate, lactate, malate, tartarate, citrate, picrate, methanesulfonate, ethanesulfonate, hydroxyethanesulfonate n_toluenesulfonate, benzenesulfonate, naphthalene sulfonate, trifluoroacetate, glutamate, aspartate, or generated pharmaceutically acceptable salts.

As used herein, the term “solvate” refers to a complex formed by the coordination of solvent molecules with the compound of the present invention in specific proportions.

As used herein, the term “hydrate” refers to a complex formed by the coordination of water molecules with the compound of the present invention.

Certain compounds of the present invention possess asymmetric carbon atoms (chiral centers) or double bonds; racemates, diastereomers, geometric isomers, regional isomers, and individual isomers (e.g., isolated enantiomers) are all encompassed within the scope of the present invention. When compounds provided herein have a defined stereochemistry (indicated as R or S, or indicated with dashed or wedge-shaped bonds), those compounds are understood by those skilled in the art to be substantially free of other isomers (e.g., at least 80%, 90%, 95%, 98%, 99%, or up to 100% free of other isomers).

The isotope-labeled compounds of the present invention refer to compounds identical to those listed herein, but wherein one or more atoms are replaced by another atom with a mass or atomic number different from the naturally occurring atom. Isotopes that can be introduced into the compound include hydrogen, carbon, nitrogen, oxygen, sulfur, such as ²H, ³H, ¹³C, ¹⁴C, ¹⁵N, ¹⁷O, ¹⁸O, ³⁵S, and the like. Compounds and their stereoisomers containing the above isotopes and/or other atomic isotopes, as well as pharmaceutically acceptable salts of the compound and stereoisomers thereof, are encompassed within the scope of the present invention. Preferably, the compound is deuterated.

### Pharmaceutical Compositions and Uses

The present invention provides a pharmaceutical composition, comprising the above compounds of Formula I, or its pharmaceutically acceptable salts, hydrates, solvates, crystal forms, and/or isotope-labeled compounds; and pharmaceutically acceptable excipients.

The compounds of the present invention have the effect of exciting, promoting, and enhancing HsClpP activity, thereby activating HsClpP to a greater extent, causing functional proteins in the mitochondria of diseased cells to be improperly degraded, reducing the level of respiratory chain proteins, and impairing oxidative phosphorylation. Therefore, the compounds of the present invention can treat corresponding diseases by acting on HsClpP to damage or kill, or regulate diseased cells.

The present invention also provides the use of the above active ingredient in the treatment of HsClpP-mediated diseases. As used herein, the term “HsClpP-mediated diseases” refers to diseases that can be treated by regulating (such as exciting and/or inhibiting) HsClpP protein. Preferably, the diseased cells in the disease exhibit abnormal expression of HsClpP (such as tumors, etc.) or mutations (such as neurological diseases, etc.).

Typical HsClpP-mediated diseases include, but are not limited to: neurological diseases, metabolic syndrome, and tumor-related diseases. Furthermore, neurological diseases include Huntington's disease, Parkinson's disease, Perrault syndrome, Alzheimer's disease, hereditary spastic paraplegia, Friedreich's ataxia, and the like. Metabolic syndrome includes diabetes, obesity, and the like. Tumors include central nervous system tumors (such as H3 K27M mutant gliomas), brain tumors, peripheral nervous system tumors, chromaffin cell tumors, paragangliomas, neuroendocrine tumors, liver cancer, lung cancer, gastric cancer, colon cancer, rectal cancer, pancreatic cancer, breast cancer, prostate cancer, endometrial cancer, hematologic malignancy, and lymphocytic malignancy, etc.

The compounds of the present invention can be administered alone or in combination with other pharmaceutically acceptable compounds. For example, the compound can be used in combination with one or more anticancer agents and/or immunosuppressants, preferably, the anticancer agents and/or immunosuppressants are selected from the group consisting of olaparib, rucaparib, niraparib, methotrexate, capecitabine, gemcitabine, doxifluridine, pemetrexed disodium, pazopanib, imatinib, erlotinib, lapatinib, gefitinib, vandetanib, herceptin, bevacizumab, rituximab, trastuzumab, paclitaxel, vinorelbine, docetaxel, doxorubicin, hydroxycamptothecin, mitomycin, epirubicin, pirarubicin, bleomycin, letrozole, tamoxifen, fulvestrant, triptorelin, flutamide, leuprorelin, anastrozole, ifosfamide, busulfan, cyclophosphamide, carmustine, nimustine, meccnu, nitrogen mustard, melphalan, chlorambucil, carboplatin, cisplatin, oxaliplatin, enloplatin, topotecan, camptothecin, topotecan, everolimus, sirolimus, temsirolimus, 6-mercaptopurine, 6-thioguanine, azathioprine, mycomycin D, daunorubicin, doxorubicin, mitoxantrone, bleomycin, mithramycin or aminoblastin, sorafenib, regorafenib; such as nivolumab, pembrolizumab, ipilimumab, avelumab, durvalumab, atezolizuma, pidilizumab, or combinations thereof.

As used herein, the term “pharmaceutically acceptable” refers to a substance that is suitable for use in humans and/or animals without excessive adverse effects (such as toxicity, irritation, and hypersensitivity), i.e., a substance with a reasonable benefit/risk ratio.

Pharmaceutically acceptable excipients, carriers, or adjuvants, which may or may not have certain physiological activity, but the addition of the ingredient does not change the dominant position of the pharmaceutical composition in the treatment of the disease, and only plays an auxiliary effect. These auxiliary effects are merely the use of the known activity of the component and are commonly-used adjuvant therapy in the field of pharmaceutical. If the above adjuvant component is used in combination with the pharmaceutical composition of the present invention, it is still within the scope of protection of the present invention.

The administration of the compounds or pharmaceutical compositions of the present invention is not particularly limited, and representative administration methods include but are not limited to oral, gastrointestinal (intravenous, intramuscular, or subcutaneous), and local administration.

Solid dosage forms for oral administration include capsules, tablets, pills, powders, and granules. In these solid dosage forms, the active compound is mixed with at least one conventional inert carrier or vehicle, such as sodium citrate or calcium phosphate, or with the following ingredients: (a) fillers or solubilizers, such as starch, lactose, sucrose, glucose, mannitol, and silicic acid; (b) binders, such as strong methylcellulose, alginate, gelatin, polyvinylpyrrolidone, sucrose and arabic gum; (c) humectants, such as glycerol; (d) disintegrants, such as agar, calcium carbonate, potato or tapioca starch, alginic acid, certain composite silicates, and sodium carbonate; (e) retarding solvent , such as carbonate, paraffin wax; (f) absorption accelerator, such as quaternary ammonium compounds; (g) wetting agents, such as cetacanol and glyceryl monostearate; (h) adsorbents, such as kaolin; (i) lubricants, such as talc, calcium stearate, magnesium stearate, solid polyethylene glycol, sodium dodecyl sulfate, or combinations thereof. Capsule, tablet, and pill formulations may also include buffering agents.

Solid dosage forms such as tablets, sugar pills, capsules, pills, and granules can be prepared using coating and shell materials, such as casings and other materials well-known in the art. They can contain opaque agents, and the release of active compounds or compounds in this composition can be delayed in a certain part of the digestive tract. Examples of embedding components that can be used are polymeric substances and waxy substances. When necessary, the active compound can also form microcapsules with one or more of the above-mentioned excipients.

Liquid dosage forms for oral administration include pharmaceutically acceptable emulsions, solutions, suspensions, syrups, or elixirs. In addition to the active compound, liquid dosage forms may contain conventional pharmaceutically acceptable inert diluents, such as water or other solvents, solubilizers, and emulsifiers, such as ethanol, isopropanol, ethyl carbonate, ethyl acetate, propylene glycol, 1,3-butanediol, dimethylformamide, and oils, especially cottonseed oil, peanut oil, corn germ oil, olive oil, castor oil, and sesame oil or mixtures of these substances.

In addition to theseinert diluents, liquid dosage forms can also contain adjuvants, such as wetting agents, emulsifying agents, suspending agents, sweetening agents, flavoring agents, and spices.

In addition to the active compounds, suspensions may contain suspending agents such as ethoxylated isooctadecanol, polyoxyethylene sorbitol and dehydrated sorbitol esters, microcrystalline cellulose, aluminum methoxide, and agar, or combinations thereof, and the like.

Compositions for parenteral injection may include physiologically acceptable sterile aqueous or anhydrous solutions, dispersions, suspensions or lotions, and sterile powders for re-dissolution into sterile injectable solutions or dispersions. Suitable aqueous and non-aqueous carriers, diluents, solvents or excipients include water, ethanol, polyols, and their suitable mixtures.

The dosage forms of the compounds of the present invention for local administration include ointments, powders, patches, sprays, and inhalers. The active ingredients are mixed with physiologically acceptable carriers and any preservatives, buffering agents, or propellants that may be necessary under sterile conditions.

The precise amount of the compound to be administered will depend on the mode of administration, the type of disease and/or condition, its severity, and the characteristics of the individual, such as general health, age, sex, weight, and tolerance to the drug. Those skilled in the art will be able to determine an appropriate dosage based on these and other factors. When administered in combination with other therapeutic agents, the “therapeutically effective amount” of any other therapeutic agent will depend on the type of drug used. Suitable doses are known for approved therapeutic agents and can be adjusted by those skilled in the art based on the condition of the individual, the type of disease, and the severity of the disease. Preferably, the compositions are formulated so that a dose of 0.01-100 mg/kg body weight/day of the compound can be administered to patients receiving these compositions. In some embodiments, the compound of the present invention provides a dose of 0.01 mg to 50 mg. In other embodiments, doses of 0.1 mg to 25 mg or 5 mg to 40 mg are provided.

Examples of subjects for administration of the pharmaceutical compositions or therapeutic agents of the present invention include mammals (e.g., humans, mice, rats, hamsters, rabbits, cats, dogs, cattle, sheep, monkeys, etc.).

The present invention also provides a therapeutic method, comprising the step of administering the compound of the present invention, or a crystal form, a pharmaceutically acceptable salt, a hydrate, a solvate, or a solvent thereof, or administering the pharmaceutical composition of the present invention to the subject in need thereof, for activating HsClpP, promoting the stability of HsClpP, and/or preventing/treating HsClpP-mediated diseases. In particular, the HsClpP-mediated related-diseases involve diseases with up-regulated/overexpressed HsClpP expression, such as cancers.

The main advantages of the present invention include:
1. The present invention provides a novel HsClpP agonist compound.
2 The compounds of the present invention exhibit excellent HsClpP agonist activity and anti-tumor activity.
3. The compounds of the present invention possess high biocompatibility and good drug forming properties.

The present invention is further described below in conjunction with specific embodiments. It is to be understood that these examples are intended to illustrate the invention only and not to limit the scope of the invention. The experimental methods in the following examples that do not specify specific conditions are usually based on conventional conditions or conditions recommended by the manufacturer. Unless otherwise specified, percentages and portions are calculated by weight.

### Preparation Method

The synthetic route for the preparation of imidazolidinopyrimidone compounds is as follows:

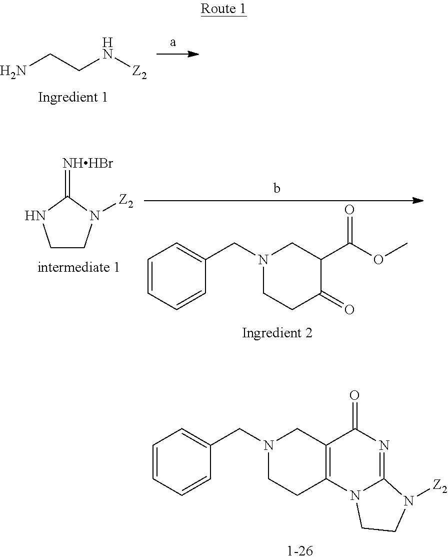

a. Intermediate 1 was obtained by reacting ingredient ingredient 1, which contains different substituents, with cyanogen bromide. IngredientIngredient 1 was dissolved in anhydrous ethanol, and cyanogen bromide was added at room temperature. After 1-24 hours of reaction, the ethanol was removed by concentration under reduced pressure, resulting in a white solid, which was intermediate 1.b. Compounds 1˜26 were obtained by cyclization of intermediate 1 and ingredient 2 under alkaline conditions. The reaction temperature was 60-100° C. The solvent used was methanol, ethanol, or n-butanol. The alkali used is sodium methoxide, sodium ethoxide, or potassium carbonate. The molar ratio of ingredient 2 to intermediate 1 and alkali was 1:1:3, and the reaction time was 3-24 hours.

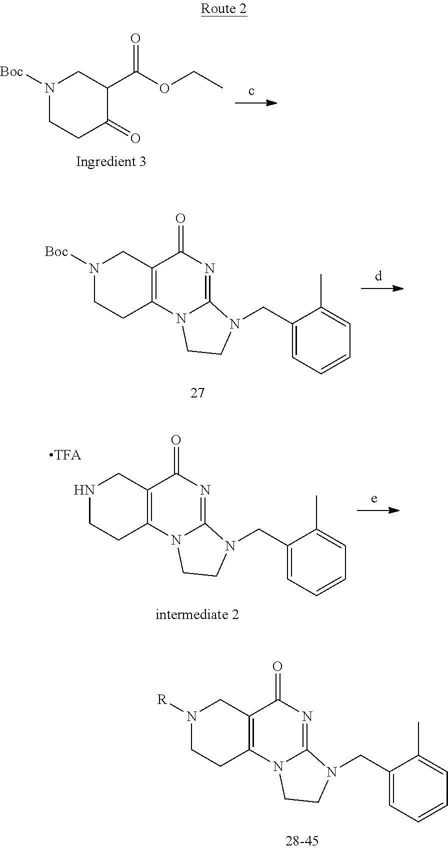

c. Compound 27 was obtained by cyclization of intermediate la and ingredient 3 under alkaline conditions. The reaction temperature was the reflux temperature of methanol, and the alkali used was sodium methoxide. The molar ratio of ingredient 3 to intermediate la and sodium methoxide was 1:1:3, and the reaction time was 3 hours.

d. Intermediate 2 was the product obtained after removing the protecting group from compound 27. The operation was carried out at room temperature by adding trifluoroacetic acid to a dichloromethane solution of intermediate 27. The reaction took 1-3 hours, then concentrated under reduced pressure to remove trifluoroacetic acid and dichloromethane, to afford the product with the protecting group removed. The volume ratio of dichloromethane to trifluoroacetic acid was 2:1.

e. Compounds 28-45 were obtained by functionalizing the nitrogen atom of intermediate 2. For compounds 27-44, intermediate 2 was dissolved in acetonitrile, and dry potassium carbonate (3 eq) and halide (1.1-1.5 eq) were sequentially added. The mixture was heated to 40-60° C. overnight. After filtration, the filtrate was concentrated under reduced pressure and went through silica gel column chromatography, eluted with methanol:dichloromethane=0:100 to 10:100, to obtain the desired product. For compound 45, intermediate 2 was dissolved in dry 1, 4-dioxane, and under nitrogen protection, aryl halide (1.5 eq) and cesium carbonate (3 eq) were added, along with a catalytic amount of Pd(dppf)Cl₂. After refluxing for 4 hours, the solvent was removed under reduced pressure, and the residue was purified by silica gel column chromatography to obtain the target product.

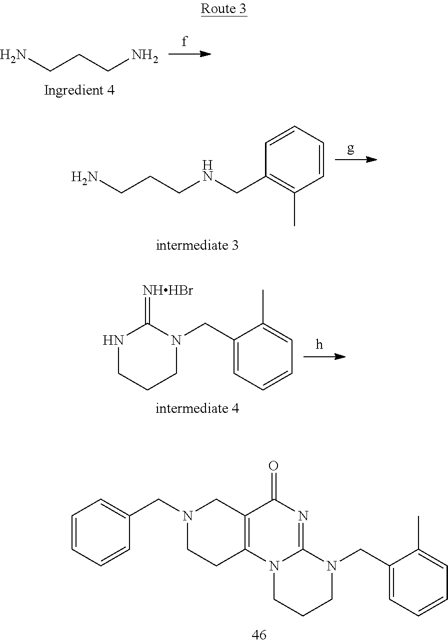

f. Intermediate 3 is prepared by functionalizing the nitrogen atom on ingredient 4. Ingredient 4 and 2-methylbenzaldehyde were dissolved in anhydrous methanol. After adding a catalytic amount of acetic acid and reacting for 1 hour, the mixture was placed under 0° C., and sodium borohydride was added. The reaction proceeded overnight at room temperature, resulting in a colorless and transparent intermediate 3.

g. Intermediate 3 was dissolved in ethanol, and cyanogen bromide was added at room temperature. After 1-24 hours of reaction, ethanol was removed under reduced pressure, resulting in a white solid, which was intermediate 4. The ratio of intermediate 3 to cyanogen bromide was 1:1.2. Compound 46 was obtained by cyclization of intermediate 4 and ingredient 2 under alkaline conditions. The reaction temperature was the reflux temperature of methanol, and the alkali used is sodium methoxide. The molar ratio of ingredient 2 to intermediate 4 and sodium methoxide was 1:1:3, and the reaction time was 3 hours.

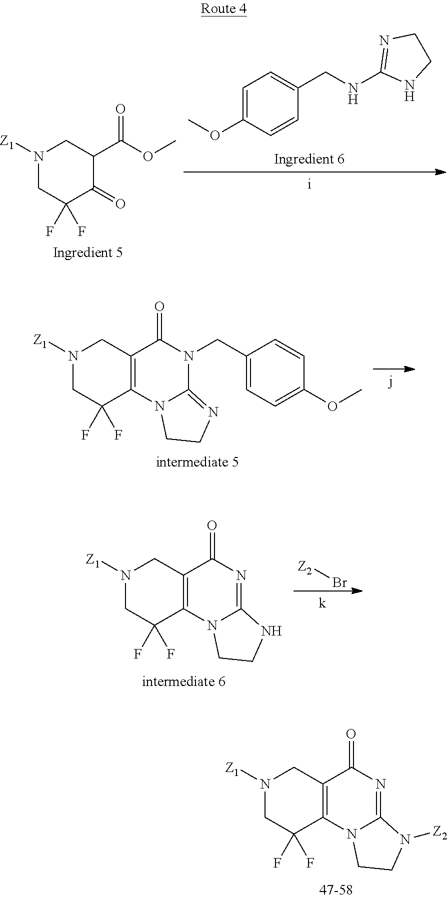

i. Intermediate 5 was obtained by cyclization of ingredient 5 and ingredient 6 under alkaline conditions. The reaction temperature was the reflux temperature of methanol, and sodium methoxide was used as the alkali. The molar ratio of ingredient 5 to ingredient 6 and sodium methoxide was 1:1:3, and the reaction time was 3 hours.

j. Intermediate 5 was treated with anhydrous aluminum trichloride, a Lewis acid, at room temperature to remove the p-methoxybenzyl group, resulting in intermediate 6. The molar ratio of intermediate 5 to anhydrous aluminum trichloride was 1:3, and the solvent used was dry dichloromethane. The reaction time was 12-24 hours. The reaction mixture was then adjusted to strong alkaline conditions with a 1M sodium hydroxide solution, and the organic phase was separated. The aqueous phase was extracted three times with chloroform:methanol (volume ratio of 10:1). The combined organic phases was dried and concentrated under reduced pressure to obtain intermediate 6.

k. Compounds 47-58 were obtained by reacting different substituted halides with intermediate 6. The bases used were any one of cesium carbonate, potassium carbonate, and triethylamine, and the solvent used were any one of DMF, DMSO, and acetonitrile. The reaction temperature was 20-60° C., and the molar ratio of intermediate 6 to halides and base was 1:1.2:3. The reaction time was 12-24 hours.

### Example 1: Synthesis of 1-(2-methylbenzyl)imidazolin-2-imine hydrobromide (Intermediate 1a)

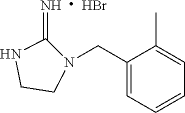

To a solution of 800 mg of oily N-(2-methylbenzyl)ethane-1,2-diamine dissolved in 30 mL of anhydrous ethanol was added cyanogen bromide (725 mg, 6.84 mmol) in batches under vigorous stirring. 5 hours later, the reaction was monitored by TLC to show that the ingredient was consumed completely. The reaction was concentrated under reduced pressure to afford a white solid. The obtained white solid was suspended and beat in ethyl acetate, after filtration, a white solid was obtained (1.1g, 4.1 mmol) with a yield of 92%. ¹H NMR (400 MHZ, DMSO-d₆) δ 8.24 (s, 2H), 8.00 (s, 1H), 7.30-7.10 (m, 4H), 4.60 (s, 2H), 3.64-3.51 (m, 2H), 3.50-3.40 (m, 2H), 2.27 (s, 3H). ¹³C NMR (101 MHZ, DMSO-d₆) δ 159.03, 136.69, 133.42, 130.96, 128.27, 127.57, 126.66, 47.53, 46.45, 41.04, 19.04.HRMS (ESI): calcd. for [M +H]+190.1344, found 190.1345.

### Example 2: Synthesis of 1-methylimidazolin-2-imine Hydrobromide (Intermediate 1b)

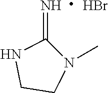

The synthesis procedure of this intermediate was the same as in Example 1, resulting in a white solid with a yield of 90%. ¹H NMR (400 MHZ, DMSO-d₆) δ 7.94 (s, 2H), 7.74 (s, 1H), 3.58 (ddd, J=9.5, 7.2, 1.7 Hz, 2H), 3.52-3.44 (m, 2H), 2.89 (s, 3H). ¹³C NMR (101 MHZ, DMSO-d₆) δ 159.40, 49.93, 41.09, 32.06.

### Example 3: Synthesis of 1-ethylimidazolin-2-imine Hydrobromide (Intermediate 1c)

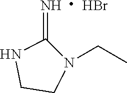

The synthesis procedure of this intermediate was the same as in Example 1, resulting in a white solid with a yield of 78%. ¹H NMR (400 MHZ, DMSO-d₆) δ 7.95 (s, 2H), 7.73 (s, 1H), 3.64-3.57 (m, 2H), 3.53-3.44 (m, 2H), 3.34 (q, J=7.2 Hz, 2H), 1.06 (t, J=7.2 Hz, 3H). ¹³C NMR (101 MHZ, DMSO-d₆) δ 158.59, 46.90, 41.01, 39.26, 12.24.

### Example 4: Synthesis of 1-Isopentylimidazolin-2-imine Hydrobromide (Intermediate 1d)

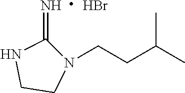

The synthesis procedure of this intermediate was the same as in Example 1, resulting in a white solid with a yield of 70%. ¹H NMR (400 MHZ, DMSO-d₆) δ 7.96 (s, 2H), 7.74 (s, 1H), 3.63-3.54 (m, 2H), 3.52-3.45 (m, 2H), 3.32-3.24 (m, 2H), 1.59-1.49 (m, 1H), 1.42-1.34 (m, 2H), 0.94 -0.84 (m, 7H).¹³C NMR (101 MHz, DMSO-d₆) δ 158.80, 47.26, 42.83, 40.97, 35.34, 25.65, 22.78.

### Example 5: Synthesis of 1-Cyclopropylmethylimidazolidin-2-imine Hydrobromide (Intermediate 1e)

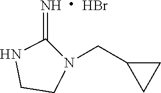

The synthesis procedure of this intermediate was the same as in Example 1, resulting in a white solid with a yield of 86%. ¹H NMR (400 MHZ, DMSO-d₆) δ 7.94 (s, 2H), 7.77 (s, 1H), 3.71 (dd, J=10.1, 7.3 Hz, 2H), 3.53 (dd, J=10.2, 7.3 Hz, 2H), 3.21 (d, J=7.1 Hz, 2H), 1.03-0.92 (m, 1H), 0.56-0.47 (m, 2H), 0.30-0.22 (m, 2H).¹³C NMR (101 MHz, DMSO-d₆) δ 158.65, 48.67, 47.58, 41.08, 9.00, 3.51.

### Example 6: Synthesis of 1-Cyclohexylmethylimidazolin-2-imine Hydrobromide (Intermediate 1f)

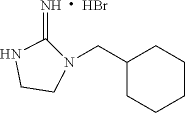

The synthesis procedure of this intermediate was the same as in Example 1, resulting in a white solid with a yield of 34%. ¹H NMR (400 MHZ, DMSO-d₆) δ 7.93 (s, 2H), 7.73 (s, 1H), 3.61 (dd, J=9.4, 6.3 Hz, 2H), 3.53 (dd, J=9.5, 6.3 Hz, 2H), 3.16 (d, J=7.1 Hz, 2H), 1.74-1.56 (m, 6H), 1.28 -1.04 (m, 3H), 1.00-0.87 (m, 2H). ¹³C NMR (101 MHZ, DMSO-d₆) δ 159.09, 50.06, 47.97, 41.00, 35.52, 29.96, 26.39, 25.61.

### Example 7: Synthesis of 1-Benzylimidazolin-2-imine Hydrobromide (Intermediate 1g)

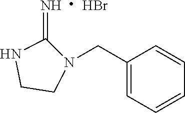

The synthesis procedure of the intermediate was the same as in Example 1, resulting in a white solid with a yield of 80%. ¹H NMR (400 MHZ, DMSO-d₆) δ 8.27 (s, 1H), 7.98 (s, 2H), 7.56-7.44 (m, 2H), 7.43-7.33 (m, 3H), 4.11-4.02 (m, 2H), 3.72-3.63 (m, 2H).¹³C NMR (101 MHZ, DMSO-d₆) δ 157.57, 136.94, 130.39, 128.14, 125.38, 51.33, 41.11.

### Example 8: Synthesis of 1-(Naphthalen-2-methyl)imidazolin-2-imine Hydrobromide (Intermediate 1h)

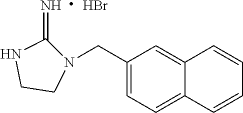

The synthesis procedure of this intermediate was the same as in Example 1, resulting in a white solid with a yield of 90%. ¹H NMR (400 MHZ, DMSO-d₆) δ 8.31 (s, 2H), 8.03-7.90 (m, 4H), 7.90-7.85 (m, 1H), 7.60-7.50 (m, 2H), 7.45 (dd, J=8.4, 1.8 Hz, 1H), 4.78 (s, 2H), 3.62-3.51 (m, 4H). ¹³C NMR (101 MHZ, DMSO-d₆) δ 159.00, 133.34, 133.13, 132.96, 129.05, 128.20, 128.11, 127.00, 126.91, 126.75, 126.18, 48.25, 47.47, 41.07.

### Example 9: Synthesis of 1-Phenylimidazolin-2-imine Hydrobromide (Intermediate li)

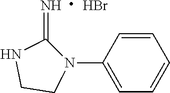

The synthesis procedure of this intermediate was the same as in Example 1, resulting in a white solid with a yield of 91%. ¹H NMR (400 MHZ, DMSO-d₆) δ 8.22 (s, 2H), 7.92 (s, 1H), 7.46-7.25 (m, 5H), 4.59 (s, 2H), 3.59-3.42 (m, 4H).¹³C NMR (101 MHz, DMSO-d₆) δ 158.94, 135.51, 129.29, 128.38, 128.27, 47.98, 47.34, 41.02.

### Example 10: Synthesis of 1-Phenylethylimidazolin-2-imine Hydrobromide (Intermediate 1j)

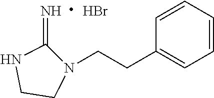

The synthesis procedure of this intermediate was the same as in Example 1, resulting in a white solid with a yield of 88%. ¹H NMR (400 MHZ, DMSO-d₆) δ 7.98 (s, 2H), 7.76 (s, 1H), 7.41-7.17 (m, 5H), 3.67-3.53 (m, 4H), 3.52-3.42 (m, 2H), 2.84 (t, J=7.5 Hz, 2H).¹³C NMR (101 MHZ, DMSO-d₆) δ 158.67, 138.54, 129.39, 128.85, 126.97, 47.55, 45.57, 41.01, 32.80.

### Example 11: Synthesis of 1-(3-methylbenzyl)imidazolin-2-imine Hydrobromide (Intermediate 1k)

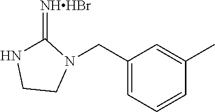

The synthesis procedure of this intermediate was the same as in Example 1, resulting in a white solid with a yield of 91%. H NMR (400 MHZ, DMSO-d₆) δ 8.22 (s, 2H), 7.91 (s, 1H), 7.32-7.25 (m, 1H), 7.18-7.03 (m, 3H), 4.55 (s, 2H), 3.56-3.41 (m, 4H), 2.30 (s, 3H). ¹³C NMR (101 MHZ, DMSO-d₆) δ 158.91, 138.50, 135.42, 129.19, 129.02, 128.84, 125.35, 47.98, 47.32, 41.01, 21.48.

### Example 12: Synthesis of 1-(4-methylbenzyl)imidazolin-2-imine Hydrobromide (Intermediate 11)

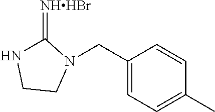

The synthesis procedure of this intermediate was the same as in Example 1, resulting in a white solid with a yield of 80%. ¹H NMR (400 MHZ, DMSO-d₆) δ 8.21 (s, 2H), 7.89 (s, 1H), 7.19 (s, 4H), 4.53 (s, 2H), 3.54-3.40 (m, 4H), 2.28 (s, 3H). ¹³C NMR (101 MHz, DMSO-d₆) δ 158.85, 137.66, 132.41, 129.82, 128.38, 47.72, 47.18, 40.99, 21.18.

### Example 13: Synthesis of 1-(2-methoxybenzyl)imidazolin-2-imine Hydrobromide (Intermediate 1m)

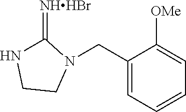

The synthesis procedure of this intermediate was the same as in Example 1, resulting in a white solid with a yield of 75%. ¹H NMR (400 MHZ, DMSO-d₆) δ 8.10 (s, 2H), 7.85 (s, 1H), 7.37-7.26 (m, 1H), 7.25-7.17 (m, 1H), 7.10-7.00 (m, 1H), 6.99-6.92 (m, 1H), 4.51 (s, 2H), 3.79 (s, 3H), 3.55-3.39 (m, 4H).¹³C NMR (101 MHz, DMSO-d₆) δ 159.00, 157.62, 129.99, 129.23, 122.93, 120.97, 111.58, 55.98, 47.47, 43.67, 40.96.

### Example 14: Synthesis of 1-(4-methoxybenzyl)imidazolin-2-imine Hydrobromide (Intermediate ln)

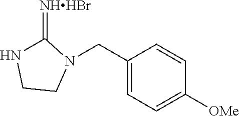

The synthesis procedure of this intermediate was the same as in Example 1, resulting in a white solid with a yield of 74%. ¹H NMR (400 MHz, DMSO-d₆) δ 8.27 (s, 2H), 7.90 (s, 1H), 7.32-7.24 (m, 2H), 7.01-6.93 (m, 2H), 4.54 (s, 2H), 3.76 (s, 3H), 3.57-3.42 (m, 4H).¹³C NMR (101 MHZ, DMSO-d₆) δ 159.45, 158.75, 129.97, 127.26, 114.64, 55.66, 47.42, 47.05, 40.97.

### Example 15: Synthesis of 1-(4-fluorobenzyl)imidazolin-2-imine Hydrobromide (Intermediate 1o)

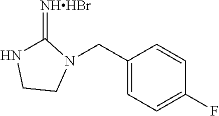

The synthesis procedure of this intermediate was the same as in Example 1, resulting in a white solid with a yield of 78%. ¹H NMR (400 MHZ, DMSO-d₆) δ 8.26 (s, 2H), 7.95 (s, 1H), 7.44-7.36 (m, 2H), 7.30-7.21 (m, 2H), 4.61 (s, 2H), 3.58-3.46 (m, 4H).¹³C NMR (101 MHZ, DMSO-d₆) 8 162.25 (d, J=243.7 Hz), 158.84, 131.74 (d, J=3.0 Hz), 130.56 (d, J=8.2 Hz), 116.09 (d, J=21.3 Hz), 47.26, 41.01.

### Example 16: Synthesis of 1-(4-chlorobenzyl)imidazolin-2-imine Hydrobromide (Intermediate 1p)

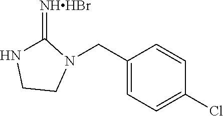

The synthesis procedure of this intermediate was the same as in Example 1, resulting in a white solid with a yield of 80%. ¹H NMR (400 MHZ, DMSO-d₆) δ 8.21 (s, 2H), 7.94 (s, 1H), 7.51-7.42 (m, 2H), 7.40-7.30 (m, 2H), 4.58 (s, 2H), 3.59-3.41 (m, 4H).¹³C NMR (101 MHZ, DMSO-d₆) 8 158.90, 134.62, 133.00, 130.28, 129.24, 47.34, 41.07.

### Example 17: Synthesis of 1-(4-bromobenzyl)imidazolin-2-imine Hydrobromide (Intermediate 1q)

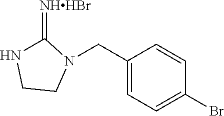

The synthesis procedure of this intermediate was the same as in Example 1, resulting in a white solid with a yield of 91%. ¹H NMR (400 MHZ, DMSO-d₆) δ 8.26 (s, 2H), 7.97 (s, 1H), 7.64-7.57 (m, 2H), 7.36-7.25 (m, 2H), 4.61 (s, 2H), 3.60-3.45 (m, 4H).¹³C NMR (101 MHz, DMSO-d₆) 8 158.90, 135.02, 132.15, 130.61, 121.55, 47.42, 47.34, 41.08.

### Example 18: Synthesis of 1-(4-trifluoromethylbenzyl)imidazolin-2-imine Hydrobromide (Intermediate 1r)

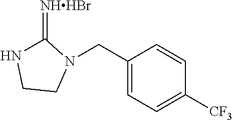

The synthesis procedure of this intermediate was the same as in Example 1, resulting in a white solid with a yield of 90%. ¹H NMR (400 MHZ, DMSO-d₆) δ 8.24 (s, 2H), 8.00 (s, 1H), 7.79 (d, J=8.0 Hz, 2H), 7.55 (d, J=8.0 Hz, 2H), 4.71 (s, 2H), 3.62-3.50 (m, 4H).¹³C NMR (101 MHZ, DMSO-d₆) δ 159.05, 140.55, 128.93, 128.89 (q, J=31.6 Hz), 126.14 (q, J=3.9 Hz), 124.66 (q, J=273.7 Hz), 47.61, 47.60, 41.09.

### Example 19: Synthesis of 1-(2, 4-difluorobenzyl)imidazolin-2-imine Hydrobromide (Intermediate 1s)

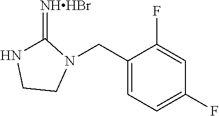

The synthesis procedure of this intermediate was the same as in Example 1, resulting in a white solid with a yield of 87%. ¹H NMR (400 MHZ, DMSO-d₆) δ 8.25 (s, 2H), 7.97 (s, 1H), 7.52 (td, J=8.6, 6.5 Hz, 1H), 7.34 (td, J=9.9, 2.6 Hz, 1H), 7.16 (td, J=8.5, 2.6 Hz, 1H), 4.66 (s, 2H), 3.52 (tt, J=9.0, 4.3 Hz, 4H).¹³C NMR (101 MHZ, DMSO-d₆) δ 163.06 (dd, J=162.6, 12.2 Hz), 160.60 (dd, J=164.3, 12.5 Hz), 158.80, 131.98 (dd, J=9.9, 5.6 Hz), 118.79 (dd, J=15.3, 3.6 Hz), 112.32 (dd, J=21.3, 3.6 Hz), 104.78 (t, J=25.7 Hz).

### Example 20: Synthesis of 1-(3,4-difluorobenzyl)imidazolin-2-imine Hydrobromide (Intermediate 1t)

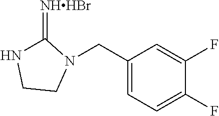

The synthesis procedure of this intermediate was the same as in Example 1, resulting in a white solid with a yield of 88%. ¹H NMR (400 MHZ, DMSO-d₆) δ 8.23 (s, 1H), 7.55-7.36 (m, 1H), 7.28-7.14 (m, 1H), 4.60 (s, 1H), 3.64-3.34 (m, 2H).¹³C NMR (101 MHz, DMSO-d₆) δ 158.88, 149.91 (dd, J=246.1, 12.8 Hz), 149.51 (dd, J=245.8, 12.4 Hz), 133.40 (J=6.7, 4.0 Hz), 125.31 (dd, J=6.7, 3.6 Hz), 118.34 (d, J=17.0 Hz), 117.58 (d, J=17.5 Hz), 47.46, 47.07, 41.05.

### Example 21: Synthesis of 1-(3-bromo-4-fluorobenzyl)imidazolin-2-imine Hydrobromide (Intermediate 1u)

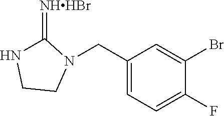

The synthesis procedure of this intermediate was the same as in Example 1, resulting in a white solid with a yield of 88%. ¹H NMR (400 MHZ, DMSO-d₆) δ 8.22 (s, 2H), 7.96 (s, 1H), 7.84-7.56 (m, 1H), 7.53-7.25 (m, 2H), 4.59 (s, 2H), 3.67-3.38 (m, 4H).¹³C NMR (101 MHz, DMSO-d₆) 8 158.85, 158.35 (d, J=245.0 Hz), 133.89 (d, J=3.7 Hz), 133.42, 129.90 (d, J=7.8 Hz), 117.54 (d, J =22.5 Hz), 108.73 (d, J=21.2 Hz), 47.45, 46.84, 41.04.

### Example 22: Synthesis of 1-(4-bromo-3-fluorobenzyl)imidazolin-2-imine Hydrobromide (Intermediate 1v)

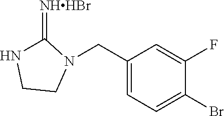

The synthesis procedure of this intermediate was the same as in Example 1, resulting in a white solid with a yield of 94%. ¹H NMR (400 MHZ, DMSO-d₆) δ 8.28 (s, 2H), 8.00 (s, 1H), 7.80-7.72 (m, 1H), 7.42 (dd, J=9.7, 2.0 Hz, 1H), 7.18 (dd, J=8.3, 2.0 Hz, 1H), 4.66 (s, 2H), 3.60-3.51 (m, 4H).¹³C NMR (101 MHz, DMSO-d₆) δ 158.94, 158.77 (d, J=245.6 Hz), 138.20 (d, J=6.5 Hz), 134.32, 125.98 (d, J=3.4 Hz), 116.65 (d, J=22.7 Hz), 107.77 (d, J=20.6 Hz), 47.55, 47.17, 41.10.

### Example 23: Synthesis of 1-(pyridin-3-methyl)imidazolin-2-imine Hydrobromide (Intermediate 1x)

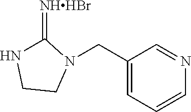

The synthesis procedure of this intermediate was the same as in Example 1, resulting in a white solid with a yield of 60%. ¹H NMR (400 MHZ, Methanol-d4) δ 8.61-8.51 (m, 2H), 7.97-7.79 (m, 1H), 7.52 (dd, J=7.9, 4.9 Hz, 1H), 4.68 (s, 2H), 3.79-3.56 (m, 4H). ¹³C NMR (101 MHZ, Methanol-d4) δ 159.16, 148.65, 148.28, 136.56, 131.32, 124.33, 47.40, 45.61, 40.71.

### Example 24: Synthesis of 1-(furan-2-methyl)imidazolin-2-imine Hydrobromide (Intermediate 1y)

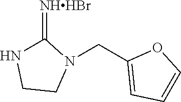

The synthesis procedure of this intermediate was the same as in Example 1, resulting in a white solid with a yield of 71%. ¹H NMR (400 MHZ, DMSO-d₆) δ 8.24 (s, 2H), 7.96 (s, 1H), 7.75-7.60 (m, 1H), 6.56-6.44 (m, 2H), 4.64 (s, 2H), 3.61-3.47 (m, 4H).¹³C NMR (101 MHZ, DMSO-d₆) 8 158.84, 148.90, 144.05, 111.09, 109.98, 47.58, 41.14, 41.01.

### Example 25: Synthesis of 1-(3,4-dichlorobenzyl)imidazolin-2-imine Hydrobromide (Intermediate 1z)

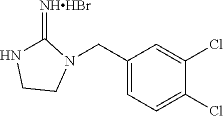

The synthesis procedure of this intermediate was the same as in Example 1, resulting in a white solid with a yield of 85%. ¹H NMR (400 MHZ, DMSO-d₆) δ 8.27 (s, 2H), 8.00 (s, 1H), 7.69 (d, J=8.2 Hz, 1H), 7.66 (d, J=2.1 Hz, 1H), 7.36 (dd, J=8.3, 2.1 Hz, 1H), 4.65 (s, 2H), 3.71-3.49 (m, 4H).¹³C NMR (101 MHz, DMSO-d₆) δ 158.92, 136.89, 131.81, 131.44, 131.02, 130.41, 128.71, 47.55, 46.99, 41.09.

### Example 26: Synthesis of 7-benzyl-3-(2-methylbenzyl)-2,3,6,7,8,9-hexahydroimidazo[1,2-a]pyrido[3,4-e]pyrimidin-5(1H)-one (Compound 1)

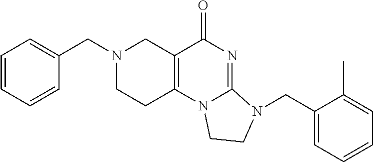 [SMILES: Cc1ccccc1CN1CCn2c1nc(=O)c1c2CCN(Cc2ccccc2)C1]

To a solution of intermediate la (1-(2-methylbenzyl)imidazolin-2-imine hydrobromide) (1 g, 3.72 mmol), and ingredient 2 (0.92 g, 3.72 mmol) in anhydrous methanol (50 mL) was added methanol sodium (500 mg, 9.3 mmol), and the mixture was heated and refluxed for 3 hours. TLC showed that the reaction was completed. The reaction was concentrated under reduced pressure to remove solvent, and the residue was suspended in water. The mixture was extracted three times with dichloromethane, and the combined organic phases were successively washed with water three times, saturated salt water once, and dried over anhydrous sodium sulfate. After filtration, the filtrate was concentrated under reduced pressure, and purified by silica gel column chromatography (eluent:

methanol:dichloromethane=1:19). The eluted fractions were combined and concentrated under reduced pressure to afford a white solid (503 mg, 1.3 mmol) with a yield of 35%. ¹H NMR (400 MHZ, DMSO-d₆) δ 7.48-7.10 (m, 9H), 4.47 (s, 2H), 4.01 (dd, J=9.7, 7.4 Hz, 2H), 3.63 (s, 2H), 3.45 (dd, J=9.7, 7.4 Hz, 2H), 3.02 (d, J=2.0 Hz, 2H), 2.66 (t, J=5.6 Hz, 2H), 2.56 (t, J=5.6 Hz, 2H), 2.28 (s, 3H). ¹³C NMR (101 MHZ, DMSO-d₆) δ 170.31, 156.69, 144.10, 138.86, 136.73, 134.77, 130.77, 129.21, 128.71, 128.45, 127.95, 127.49, 126.40, 109.17, 62.00, 49.92, 48.90, 45.94, 44.81, 42.45, 25.06, 19.17, 19.14.HRMS (ESI): calcd. for [M+Na]⁺409.2004, found 409.2001.

### Example 27: Synthesis of 7-benzyl-3-methyl-2,3,6,7,8,9-hexahydroimidazo[1,2-a]pyrido[3,4-e]pyrimidin-5(1H)-one (Compound 2)

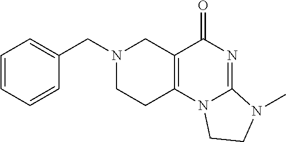 [SMILES: CN1CCn2c1nc(=O)c1c2CCN(Cc2ccccc2)C1]

Apart from the substitution of the key intermediate, the synthesis steps of the target product were the same as in Example 26, resulting in a white solid with a yield of 37%. . ¹H NMR (400 MHZ, DMSO-d₆) δ 7.38-7.22 (m, 5H), 3.97 (dd, J=9.6, 7.4 Hz, 2H), 3.62 (s, 2H), 3.56 (dd, J=9.4, 7.6 Hz, 2H), 2.99 (d, J=3.8 Hz, 2H), 2.81 (s, 3H), 2.69-2.60 (m, 2H), 2.57-2.52 (m, 2H). ¹³C NMR (101 MHZ, Methanol-d4) δ 172.36, 157.07, 145.39, 137.20, 129.23, 128.08, 127.18, 108.14, 61.84, 49.23, 47.91, 46.83, 42.27, 30.16, 24.57.HRMS (ESI⁺): m/z calcd for C₁₇H₂₁N₄O [M+H]⁺297.1715, found 297.1703.

### Example 28: Synthesis of 7-benzyl-3-ethyl-2,3,6,7,8,9-hexahydroimidazo[1,2-a]pyrido[3,4-e]pyrimidin-5(1H)-one (Compound 3)

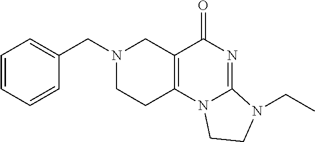 [SMILES: CCN1CCn2c1nc(=O)c1c2CCN(Cc2ccccc2)C1]

Apart from the substitution of the key intermediate, the synthesis steps of the target product were the same as in Example 26, resulting in a white solid with a yield of 35%. ¹H NMR (400 MHZ, DMSO-d₆) δ 7.38-7.21 (m, 5H), 3.97 (dd, J=9.7, 7.4 Hz, 2H), 3.61 (s, 2H), 3.57 (dd, J=9.7, 7.4 Hz, 2H), 3.29 (q, J=7.2 Hz, 2H), 2.99 (d, J=3.7 Hz, 2H), 2.67-2.60 (m, 2H), 2.55-2.50 (m, 2H), 1.07 (t, J=7.2 Hz, 3H). ¹³C NMR (101 MHZ, DMSO-d₆) δ 170.13, 156.60, 143.72, 138.86, 129.22, 128.70, 127.49, 109.00, 62.01, 49.94, 48.92, 44.18, 42.33, 38.71, 24.99, 12.20.HRMS (ESI⁺): m/z calcd for C₁₈H₂₃N₄O [M+H]⁺311.1872, found 311.1856.

### Example 29: Synthesis of 7-benzyl-3-isopentyl-2,3,6,7,8,9-hexahydroimidazo[1,2-a]pyrido[3,4-e]pyrimidin-5(1H)-one (Compound 4)

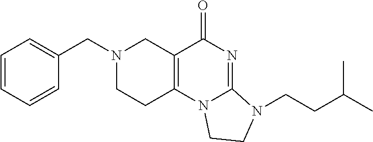 [SMILES: CC(C)CCN1CCn2c1nc(=O)c1c2CCN(Cc2ccccc2)C1]

Apart from the substitution of the key intermediate, the synthesis steps of the target product were the same as in Example 26 resulting in a white solid with a yield of 61%. ¹H NMR (400 MHZ, Chloroform-d) δ 7.36-7.27 (m, 4H), 7.26-7.22 (m, 1H), 3.92 (dd, J=9.7, 7.3 Hz, 2H), 3.69 (s, 2H), 3.62 (dd, J=9.8, 7.2 Hz, 2H), 3.48-3.39 (m, 4H), 2.68 (t, J=5.7 Hz, 2H), 2.49 (t, J=5.8 Hz, 2H), 1.68-1.55 (m, 1H), 1.50-1.39 (m, 2H), 0.93 (s, 3H), 0.91 (s, 3H).¹³C NMR (101 MHZ, DMSO-d₆) δ 170.13, 156.82, 143.69, 138.88, 129.20, 129.00, 128.70, 127.48, 108.99, 62.02, 49.96, 48.92, 44.67, 42.33, 35.57, 25.67, 25.01, 22.83.HRMS (ESI⁺): m/z calcd for C₂₁H₂₉N₄O [M+H]⁺353.2341, found 353.2330.

### Example 30: Synthesis of 7-benzyl-3-cyclopropylmethyl-2,3,6,7,8,9-hexahydroimidazo[1,2-a]pyrido[3,4-e]pyrimidin-5(1H)-one (Compound 5)

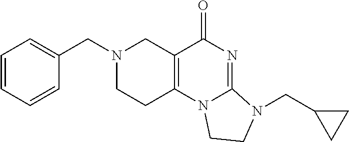 [SMILES: O=c1nc2n(c3c1CN(Cc1ccccc1)CC3)CCN2CC1CC1]

Apart from the substitution of the key intermediate, the synthesis steps of the target product were the same as in Example 26, resulting in a white solid with a yield of 32%. ¹H NMR (400 MHZ, DMSO-d₆) δ 7.37-7.21 (m, 5H), 3.99 (dd, J=9.7, 7.3 Hz, 2H), 3.67 (dd, J=9.8, 7.3 Hz, 2H), 3.61 (s, 2H), 3.12 (d, J=7.0 Hz, 2H), 2.99 (s, 2H), 2.67-2.59 (m, 2H), 2.56-2.50 (m, 2H), 1.01-0.87 (m, 1H), 0.51-0.42 (m, 2H), 0.25-0.17 (m, 2H).¹³C NMR (101 MHz, DMSO-d₆) δ 170.10, 156.76, 143.73, 138.86, 129.22, 128.71, 127.49, 109.07, 62.01, 49.94, 48.91, 48.51, 44.95, 42.45, 25.00, 9.05, 3.58.HRMS (ESI⁺): m/z calcd for C₂₀H₂₅N₄O [M+H] 337.2028, found 337.2015.

### Example 31: Synthesis of 7-benzyl-3-cyclohexylmethyl-2,3,6,7,8,9-hexahydroimidazo[1,2-a]pyrido[3,4-e]pyrimidin-5(1H)-one (Compound 6)

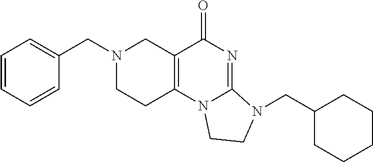 [SMILES: O=c1nc2n(c3c1CN(Cc1ccccc1)CC3)CCN2CC1CCCCC1]

Apart from the substitution of the key intermediate, the synthesis steps of the target product were the same as in Example 26, resulting in awhite solid with a yield of 37%. ¹H NMR (400 MHZ, DMSO-d₆) δ 7.41-7.19 (m, 5H), 3.99 (dd, J=9.8, 7.4 Hz, 2H), 3.61 (s, 2H), 3.58 (dd, J=9.8, 7.3 Hz, 2H), 3.09 (d, J=7.0 Hz, 2H), 2.98 (s, 2H), 2.72-2.58 (m, 2H), 2.57-2.51 (m, 2H), 1.74-1.54 (m, 6H), 1.27-1.07 (m, 3H), 0.99-0.83 (m, 2H).¹³C NMR (101 MHz, DMSO-d₆) δ 170.14, 157.14, 143.65, 138.89, 129.19, 128.70, 127.48, 108.95, 62.03, 50.15, 49.98, 48.96, 45.54, 42.37, 35.71, 30.61, 26.50, 25.70, 25.03.HRMS (ESI⁺): m/z calcd for C₂₃H₃₁N₄O [M+H]⁺379.2498, found 379.2498.

### Example 32: Synthesis of 7-benzyl-3-benzyl-2,3,6,7,8,9-hexahydroimidazo[1,2-a]pyrido[3,4-e]pyrimidin-5(1H)-one (Compound 7)

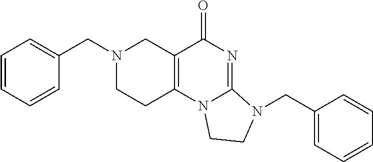 [SMILES: O=c1nc2n(c3c1CN(Cc1ccccc1)CC3)CCN2Cc1ccccc1]

Apart from the substitution of the key intermediate, the synthesis steps of the target product were the same as in Example 26, resulting in a white solid with a yield of 40%. ¹H NMR (400 MHZ, Methanol-d4) δ 7.80-7.68 (m, 2H), 7.46-7.28 (m, 7H), 7.17-7.08 (m, 1H), 4.18 (s, 4H), 3.72 (s, 2H), 3.29-3.26 (m, 2H), 2.79-2.73 (m, 2H), 2.70-2.62 (m, 2H). ¹³C NMR (101 MHZ, Chloroform-d) δ 171.10, 156.86, 142.47, 142.43, 137.84, 129.11, 128.34, 127.26, 110.29, 110.25, 62.32, 50.16, 48.04, 46.99, 42.20, 31.48, 25.43.HRMS (ESI⁺): m/z calcd for C₂₂H₂₃N₄O [M+H]⁺359.1872, found 359.1859.

### Example 33: Synthesis of 7-benzyl-3-(2-naphthylmethyl)-2,3,6,7,8,9-hexahydroimidazo[1,2-a]pyrido[3,4-e]pyrimidin-5(1H)-one (Compound 8)

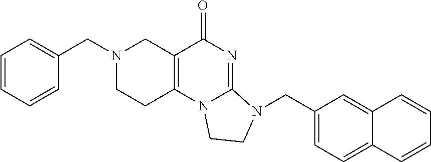 [SMILES: O=c1nc2n(c3c1CN(Cc1ccccc1)CC3)CCN2Cc1ccc2ccccc2c1]

Apart from the substitution of the key intermediate, the synthesis steps of the target product were the same as in Example 26, resulting in a white solid with a yield of 41%. ¹H NMR (400 MHZ, DMSO-d₆) δ 7.90 (dd, J=8.7, 4.6 Hz, 3H), 7.83 (d, J=1.7 Hz, 1H), 7.55-7.42 (m, 3H), 7.37 -7.23 (m, 5H), 4.65 (s, 2H), 4.01 (dd, J=9.7, 7.5 Hz, 2H), 3.63 (s, 2H), 3.52 (dd, J=9.7, 7.3 Hz, 2H), 3.04 (d, J=2.0 Hz, 2H), 2.72-2.61 (m, 2H), 2.60-2.51 (m, 2H). ¹³C NMR (101 MHz, DMSO-d₆) 8 170.13, 156.90, 143.87, 138.88, 134.71, 133.38, 132.87, 129.22, 128.78, 128.72, 128.10, 128.07, 127.50, 126.82, 126.80, 126.49, 126.45, 109.41, 62.03, 49.99, 48.92, 47.75, 44.63, 42.43, 25.05.HRMS (ESI⁺): m/z calcd for C₂₇H₂₇N₄O [M+H]⁺423.2185, found 423.2186.

### Example 34: Synthesis of 7-benzyl-3-phenyl-2,3,6,7,8,9-hexahydroimidazo[1,2-a]pyrido[3,4-e]pyrimidin-5(1H)-one (Compound 9)

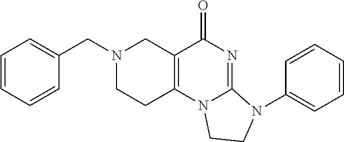 [SMILES: O=c1nc2n(c3c1CN(Cc1ccccc1)CC3)CCN2c1ccccc1]

Apart from the substitution of the key intermediate, the synthesis steps of the target product were the same as in Example 26, resulting in a white solid with a yield of 41%. ¹H NMR (400 MHZ, DMSO-d₆) δ 7.43-7.20 (m, 10H), 4.48 (s, 2H), 4.00 (dd, J=9.7, 7.4 Hz, 2H), 3.63 (s, 2H), 3.48 (dd, J=9.7, 7.4 Hz, 2H), 3.02 (s, 2H), 2.69-2.60 (m, 2H), 2.58-2.52 (m, 2H). ¹³C NMR (101 MHZ, DMSO-d₆) δ 170.11, 156.85, 143.86, 138.88, 137.02, 129.22, 129.06, 128.71, 128.33, 127.93, 127.50, 109.34, 62.01, 49.97, 48.92, 47.50, 44.54, 42.38, 25.02.HRMS (ESI⁺): m/z calcd for C₂₃H₂₅N₄O [M+H]⁺373.2028, found 373.2014.

### Example 35: Preparation of 7-benzyl-3-(phenylethyl)-2,3,6,7,8,9-hexahydroimidazo[1,2-a]pyrido[3,4-e]pyrimidin-5(1H)-one (Compound 10)

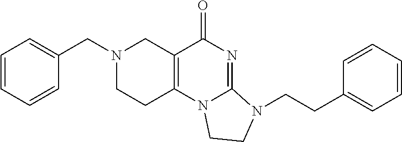 [SMILES: O=c1nc2n(c3c1CN(Cc1ccccc1)CC3)CCN2CCc1ccccc1]

Apart from the substitution of the key intermediate, the synthesis steps of the target product were the same as in Example 26, resulting in a white solid with a yield of 35%. ¹H NMR (400 MHZ, DMSO-d₆) δ 7.40-7.15 (m, 10H), 3.94 (dd, J=9.7, 7.4 Hz, 2H), 3.60 (s, 2H), 3.54 (dd, J=9.7, 7.4 Hz, 2H), 3.49 (dd, J=8.4, 6.6 Hz, 2H), 2.99 (s, 2H), 2.88-2.76 (m, 2H), 2.66-2.57 (m, 2H), 2.54-2.49 (m, 2H). ¹³C NMR (101 MHz, DMSO-d₆) δ 170.12, 156.74, 143.75, 139.44, 138.85, 129.22, 129.10, 128.90, 128.71, 127.50, 126.74, 109.13, 62.01, 49.94, 48.89, 45.55, 45.07, 42.37, 33.04, 24.99.HRMS (ESI⁺): m/z calcd for C₂₄H₂₇N₄O [M+H]⁺387.2185, found 387.2183.

### Example 36: Preparation of 7-benzyl-3-(3-methylbenzyl)-2,3,6,7,8,9-hexahydroimidazo[1,2-a]pyrido[3,4-e]pyrimidin-5(1H)-one (Compound 11)

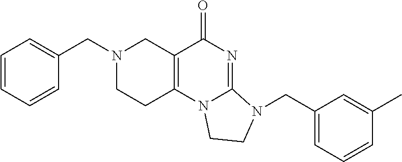 [SMILES: Cc1cccc(CN2CCn3c2nc(=O)c2c3CCN(Cc3ccccc3)C2)c1]

Apart from the substitution of the key intermediate, the synthesis steps of the target product were the same as in Example 26, resulting in a white solid with a yield of 36%. ¹H NMR (400 MHZ, DMSO-d₆) δ 7.37-7.20 (m, 6H), 7.15-7.03 (m, 3H), 4.44 (s, 2H), 3.99 (dd, J=9.7, 7.4 Hz, 2H), 3.62 (s, 2H), 3.47 (dd, J=9.8, 7.3 Hz, 2H), 3.03 (d, J=2.0 Hz, 2H), 2.69-2.59 (m, 2H), 2.58 -2.51 (m, 2H), 2.29 (s, 3H). ¹³C NMR (101 MHz, DMSO-d₆) δ 170.11, 156.81, 143.84, 138.88, 138.23, 136.95, 129.22, 128.97, 128.90, 128.71, 128.59, 127.49, 125.42, 109.32, 62.02, 49.98, 48.91, 47.50, 44.53, 42.36, 25.03, 21.50, 21.46.HRMS (ESI⁺): m/z calcd for C₂₄H₂₇N₄O [M+H] 387.2185, found 387.2170.

### Example 37: Preparation of 7-benzyl-3-(4-methylbenzyl)-2,3,6,7,8,9-hexahydroimidazo[1,2-a]pyrido[3,4-e]pyrimidin-5(1H)-one (Compound 11)

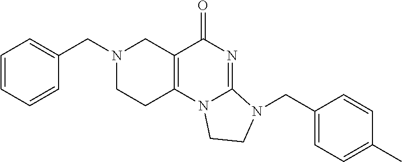 [SMILES: Cc1ccc(CN2CCn3c2nc(=O)c2c3CCN(Cc3ccccc3)C2)cc1]

Apart from the substitution of the key intermediate, the synthesis steps of the target product were the same as in Example 26, resulting in a white solid with a yield of 36%. ¹H NMR (400 MHZ, DMSO-d₆) δ 7.39-7.22 (m, 5H), 7.22-7.09 (m, 4H), 4.43 (s, 2H), 3.99 (dd, J=9.7, 7.4 Hz, 2H), 3.63 (s, 2H), 3.45 (dd, J=9.7, 7.4 Hz, 2H), 3.02 (s, 2H), 2.74-2.59 (m, 2H), 2.58-2.49 (m, 4H), 2.28 (s, 3H). ¹³C NMR (101 MHz, DMSO-d₆) δ 170.11, 156.82, 143.86, 138.88, 137.12, 133.90, 129.62, 129.22, 128.72, 128.40, 127.49, 109.30, 62.00, 49.95, 48.92, 47.21, 44.39, 42.35, 26.41, 22.12.HRMS (ESI⁺): m/z calcd for C₂₄H₂₇N₄O [M+H]⁺387.2185, found 387.2171.

### Example 38: Preparation of 7-benzyl-3-(2-methoxybenzyl)-2,3,6,7,8,9-hexahydroimidazo[1,2-a]pyrido[3,4-e]pyrimidin-5(1H)-one (Compound 13)

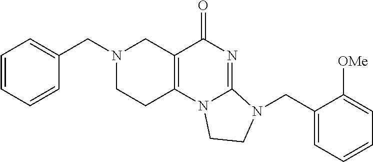 [SMILES: COc1ccccc1CN1CCn2c1nc(=O)c1c2CCN(Cc2ccccc2)C1]

Apart from the substitution of the key intermediate, the synthesis steps of the target product were the same as in Example 26, resulting in a white solid with a yield of 61%. ¹H NMR (400 MHZ, DMSO-d₆) δ 7.36-7.16 (m, 7H), 7.06-6.97 (m, 1H), 6.94-6.87 (m, 1H), 4.44 (s, 2H), 3.99 (dd, J=9.8, 7.4 Hz, 2H), 3.80 (s, 3H), 3.61 (s, 2H), 3.52 (dd, J=9.8, 7.4 Hz, 2H), 3.00 (s, 2H), 2.68-2.59 (m, 2H), 2.58-2.51 (m, 2H).¹³C NMR (101 MHZ, DMSO-d₆) δ 170.14, 157.56, 156.91, 143.82, 138.87, 129.22, 128.89, 128.70, 127.49, 124.60, 120.83, 111.30, 109.19, 62.02, 55.88, 49.97, 48.91, 45.14, 42.69, 42.40, 25.02.HRMS (ESI⁺): m/z calcd for C₂₄H₂₇N₄O₂[M+H]⁺403.2134, found 403.2144.

### Example 39: Preparation of 7-benzyl-3-(4-methoxybenzyl)-2,3,6,7,8,9-hexahydroimidazo[1,2-a]pyrido[3,4-e]pyrimidin-5(1H)-o ne (Compound 14)

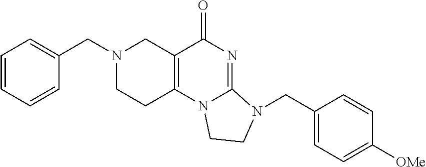 [SMILES: COc1ccc(CN2CCn3c2nc(=O)c2c3CCN(Cc3ccccc3)C2)cc1]

Apart from the substitution of the key intermediate, the synthesis steps of the target product were the same as in Example 26, resulting in a white solid with a yield of 30%. ¹H NMR (400 MHZ, DMSO-d₆) δ 7.39-7.20 (m, 7H), 6.95-6.88 (m, 2H), 4.40 (s, 2H), 3.97 (dd, J=9.7, 7.4 Hz, 2H), 3.73 (s, 3H), 3.62 (s, 2H), 3.44 (dd, J=9.7, 7.3 Hz, 2H), 3.02 (s, 2H), 2.69-2.60 (m, 2H), 2.57 -2.51 (m, 2H).¹³C NMR (101 MHz, DMSO-d₆) δ 170.09, 159.19, 156.78, 143.82, 138.88, 129.87, 129.22, 128.78, 128.71, 127.49, 114.44, 109.30, 62.01, 55.57, 55.53, 49.97, 48.91, 46.91, 44.29, 42.33, 25.00.HRMS (ESI⁺): m/z calcd for C₂₄H₂₇N₄O₂[M+H]⁺403.2134, found 403.2184.

### Example 40: Preparation of 7-benzyl-3-(4-fluorobenzyl)-2,3,6,7,8,9-hexahydroimidazo[1,2-a]pyrido[3,4-e]pyrimidin-5(1H)-one (Compound 15)

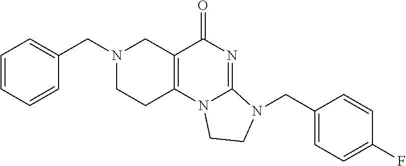 [SMILES: O=c1nc2n(c3c1CN(Cc1ccccc1)CC3)CCN2Cc1ccc(F)cc1]

Apart from the substitution of the key intermediate, the synthesis steps of the target product were the same as in Example 26, resulting in a white solid with a yield of 34%. ¹H NMR (400 MHZ, DMSO-d₆) δ 7.44-7.23 (m, 7H), 7.22-7.14 (m, 2H), 4.47 (s, 2H), 4.00 (dd, J=9.7, 7.4 Hz, 2H), 3.63 (s, 2H), 3.48 (dd, J=9.7, 7.4 Hz, 2H), 3.03 (d, J=2.0 Hz, 2H), 2.72-2.61 (m, 2H), 2.57 -2.51 (m, 2H).¹³C NMR (101 MHZ, DMSO-d₆) δ 170.07, 163.25, 160.84, 156.79, 143.86, 138.87, 133.27, 133.24, 130.48, 130.40, 129.21, 128.71, 127.49, 115.92, 115.71, 109.39, 62.01, 49.96, 48.90, 46.76, 44.48, 42.39, 25.02.HRMS (ESI⁺): m/z calcd for C₂₃H₂₄FN₄O [M+H]⁺391.1934, found 391.1936.

### Example 41: Preparation of 7-benzyl-3-(4-chlorobenzyl)-2,3,6,7,8,9-hexahydroimidazo[1,2-a]pyrido[3,4-e]pyrimidin-5(1H)-one (Compound 16)

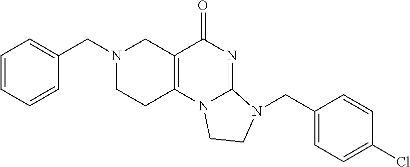 [SMILES: O=c1nc2n(c3c1CN(Cc1ccccc1)CC3)CCN2Cc1ccc(Cl)cc1]

Apart from the substitution of the key intermediate, the synthesis steps of the target product were the same as in Implementation Example 26, resulting in a white solid with a yield of 40%. 1H NMR (400 MHZ, Chloroform-d) δ 7.36-7.19 (m, 9H), 4.59-4.53 (m, 2H), 3.93-3.83 (m, 2H), 3.70-3.65 (m, 2H), 3.52-3.42 (m, 2H), 3.42-3.36 (m, 2H), 2.72-2.64 (m, 2H), 2.52-2.44 (m, 2H). ¹³C NMR (101 MHz, Chloroform-d) δ 171.06, 156.49, 142.56, 142.50, 137.85, 134.33, 133.82, 129.93, 129.09, 128.92, 128.37, 127.29, 111.13, 111.03, 62.29, 50.20, 48.04, 47.40, 43.93, 42.29, 25.53.HRMS (ESI⁺): m/z calcd for C₂₃H₂₄Cl₄O [M+H]⁺407.1639, found 407.1630.

### Example 42: Preparation of 7-benzyl-3-(4-bromobenzyl)-2,3,6,7,8,9-hexahydroimidazo[1,2-a]pyrido[3,4-e]pyrimidin-5(1H)-one

(Compound 17)

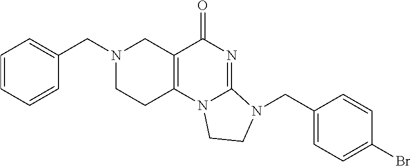 [SMILES: O=c1nc2n(c3c1CN(Cc1ccccc1)CC3)CCN2Cc1ccc(Br)cc1]

Apart from the substitution of the key intermediate, the synthesis steps of the target product were the same as in Implementation Example 26, resulting in a white solid with a yield of 32%. 1H NMR (400 MHZ, DMSO-d₆) δ 7.58-7.51 (m, 1H), 7.36-7.23 (m, 3H), 4.45 (s, 1H), 4.02-3.97 (m, 1H), 3.62 (s, 1H), 3.48 (dd, J=9.8, 7.3 Hz, 1H), 3.01 (s, 1H), 2.68-2.59 (m, 1H), 2.58-2.51 (m, 1H).¹³C NMR (101 MHz, Chloroform-d) δ 171.00, 156.47, 142.44, 137.81, 134.83, 131.87, 130.25, 129.08, 128.35, 127.28, 121.92, 111.10, 62.27, 50.18, 48.02, 47.44, 43.91, 42.27, 25.52.HRMS (ESI⁺): m/z calcd for C₂₃H₂₄BrN₄O [M+H]⁺451.1133, found 451.1125.

### Example 43: Preparation of 7-benzyl-3-(4-trifluoromethylbenzyl)-2,3,6,7,8,9-hexahydroimidazo[1,2-a]pyrido[3,4-e]pyrimidin-5( 1H)-one (Compound 18)

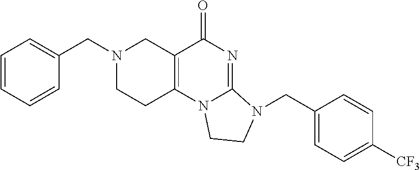 [SMILES: O=c1nc2n(c3c1CN(Cc1ccccc1)CC3)CCN2Cc1ccc(C(F)(F)F)cc1]

Apart from the substitution of the key intermediate, the synthesis steps of the target product were the same as in Example 26, resulting in a white solid with a yield of 32%. ¹H NMR (400 MHZ, DMSO-d₆) δ 7.73 (d, 2H), 7.54 (d, J=7.7 Hz, 2H), 7.43-7.15 (m, 5H), 4.58 (s, 2H), 4.03 (t, J=8.5 Hz, 2H), 3.63 (s, 2H), 3.53 (t, J=8.5 Hz, 2H), 3.02 (s, 2H), 2.76-2.62 (m, 2H), 2.60-2.53 (m, 2H). ¹³C NMR (101 MHZ, Chloroform-d) δ 169.98, 155.53, 141.52, 138.97, 136.77, 129.15 (q, J=32.4 Hz), 128.08, 127.72, 127.36, 126.31, 123.00 (q, J=272.2 Hz), 123.00 (d, J=272.2 Hz), 61.28, 49.16, 47.05, 46.66, 43.09, 41.29, 24.51.HRMS (ESI⁺): m/z calcd for C₂₄H₂₄N₄O [M+H]⁺441.1902, found 441.1901.

### Example 44: Preparation of 7-benzyl-3-(2, 4-difluorobenzyl)-2,3,6,7,8,9-hexahydroimidazo[1,2-a]pyrido[3,4-e]pyrimidin-5(1H)-one (Compound 19).

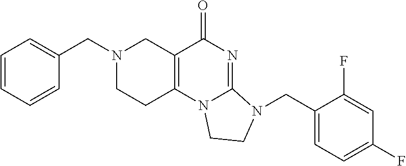 [SMILES: O=c1nc2n(c3c1CN(Cc1ccccc1)CC3)CCN2Cc1ccc(F)cc1F]

Apart from the substitution of the key intermediate, the synthesis steps of the target product were the same as in Example 26, resulting in a white solid with a yield of 38%. ¹H NMR (400 MHZ,

DMSO-d₆) δ 7.49-7.40 (m, 1H), 7.37-7.23 (m, 6H), 7.13-7.04 (m, 1H), 4.51 (s, 2H), 4.01 (dd, J =9.7, 7.4 Hz, 2H), 3.62 (s, 2H), 3.52 (dd, J=9.7, 7.3 Hz, 2H), 3.01 (d, J=1.9 Hz, 2H), 2.69-2.60 (m, 2H), 2.58-2.52 (m, 2H).¹³C NMR (101 MHz, DMSO-d₆) δ 169.99, 162.30 (dd, J=245.9, 12.3 Hz), 160.95 (dd, J=247.9, 12.5 Hz), 156.70, 143.87, 138.86, 132.08 (dd, J=9.8, 5.8 Hz), 129.21, 128.71, 127.49, 120.21 (dd, J=15.4, 3.7 Hz), 112.11 (d, J=20.9 Hz), 109.46, 104.45 (t, J=25.4

Hz), 61.99, 49.93, 48.89, 44.82, 42.44, 41.21, 25.01.HRMS (ESI⁺): m/z calcd for C₂₃H₂₃F₂N₄O [M+H]⁺409.1840, found 409.1823.

### Example 45: Preparation of 7-benzyl-3-(3,4-difluorobenzyl)-2,3,6,7,8,9-hexahydroimidazo[1,2-a]pyrido[3,4-e]pyrimidin-5(1H)-one (Compound 20)

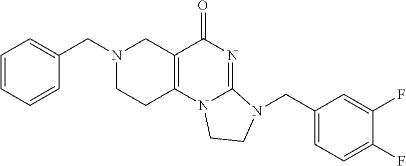 [SMILES: O=c1nc2n(c3c1CN(Cc1ccccc1)CC3)CCN2Cc1ccc(F)c(F)c1]

Apart from the substitution of the key intermediate, the synthesis steps of the target product were the same as in Example 26, resulting in a white solid with a yield of 31%. ¹H NMR (400 MHZ, DMSO-d₆) δ 7.46-7.13 (m, 8H), 4.47 (s, 2H), 4.02 (dd, J=9.7, 7.4 Hz, 2H), 3.64 (s, 2H), 3.52 (dd, J=9.8, 7.3 Hz, 2H), 3.04 (s, 2H), 2.73-2.61 (m, 2H), 2.60-2.52 (m, 2H). ¹³C NMR (101 MHZ, DMSO-d₆) δ 170.04, 156.77, 149.87 (dd, J=245.8, 12.6 Hz), 149.25 (dd, J=244.6, 12.6 Hz), 143.89, 138.75, 135.05 (t, J=4.4 Hz), 129.24, 128.72, 127.54, 125.07, 118.06 (d, J=17.0 Hz), 117.28 (d, J =17.1 Hz), 109.34, 61.95, 49.90, 48.88, 46.54, 44.68, 42.48, 24.99.HRMS (ESI⁺): m/z calcd for C₂₃H₂₃F₂N₄O [M+H]⁺409.1840, found 409.1823.

### Example 46: Preparation of 7-benzyl-3-(3-bromo-4-fluorobenzyl)-2,3,6,7,8,9-hexahydroimidazo[1,2-a]pyrido[3,4-e]pyrimidin-5 (1H)-one (Compound 21)

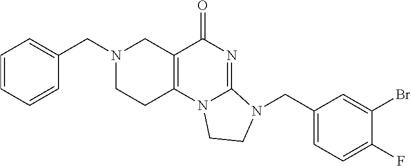 [SMILES: O=c1nc2n(c3c1CN(Cc1ccccc1)CC3)CCN2Cc1ccc(F)c(Br)c1]

Apart from the substitution of the key intermediate, the synthesis steps of the target product were the same as in Example 26, resulting in a white solid with a yield of 35%. ¹H NMR (400 MHZ, DMSO-d₆) δ 7.71-7.61 (m, 1H), 7.41-7.21 (m, 7H), 4.47 (s, 2H), 4.01 (dd, J=9.7, 7.4 Hz, 2H), 3.63 (s, 2H), 3.51 (dd, J=9.7, 7.3 Hz, 2H), 3.02 (d, J=1.9 Hz, 2H), 2.72-2.61 (m, 2H), 2.59 -2.52 (m, 2H). ¹³C NMR (101 MHz, DMSO-d₆) δ 170.02, 158.09 (d, J=244.3 Hz), 156.76, 143.90, 138.87, 135.54 (d, J=3.6 Hz), 133.17, 129.73 (d, J=7.6 Hz), 129.21, 128.71, 127.49, 117.30 (d, J=22.1 Hz), 109.44, 108.49 (d, J=21.2 Hz), 62.01, 49.95, 48.90, 46.35, 44.68, 42.46, 25.04.HRMS (ESI⁺): m/z calcd for C₂₃H₂₃BrFN₄O [M+H]⁺469.1039, found 469.1040.

### Example 47: Preparation of 7-benzyl-3-(4-bromo-3-fluorobenzyl)-2,3,6,7,8,9-hexahydroimidazo[1,2-a]pyrido[3,4-e]pyrimidin-5 (1H)-one (Compound 22)

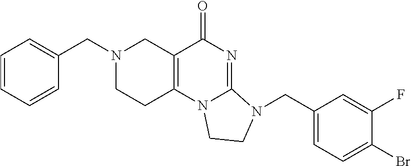 [SMILES: O=c1nc2n(c3c1CN(Cc1ccccc1)CC3)CCN2Cc1ccc(Br)c(F)c1]

Apart from the substitution of the key intermediate, the synthesis steps of the target product were the same as in Example 26, resulting in a white solid with a yield of 33%. ¹H NMR (400 MHZ,

Chloroform-d) δ 7.48 (t, J=7.6 Hz, 1H), 7.38-7.29 (m, 4H), 7.26 (dd, J=5.7, 2.9 Hz, 1H), 7.08 (dd, J=9.1, 2.0 Hz, 1H), 6.99 (dd, J=8.2, 2.0 Hz, 1H), 4.56 (s, 2H), 3.92 (dd, J=9.7, 7.4 Hz, 2H), 3.69 (s, 2H), 3.51 (dd, J=9.7, 7.3 Hz, 2H), 3.38 (d, J=2.0 Hz, 2H), 2.69 (t, J=5.7 Hz, 2H), 2.50 (t,

J=5.8 Hz, 2H).¹³C NMR (101 MHZ, Chloroform-d) § 170.95, 159.14 (d, J=248.8 Hz), 156.46, 142.50, 137.80, 137.74, 133.84, 129.11, 128.38, 127.32, 125.29, 116.39 (d, J=23.2 Hz), 111.29, 108.43 (d, J=21.2 Hz), 62.28, 50.16, 48.03, 47.24, 44.09, 42.33, 25.54.HRMS (ESI⁺): m/z calcd for C₂₃H₂₃BrFN₄O [M+H]⁺469.1039, found 469.1037.

### Example 48: Preparation of 7-benzyl-3-(3-pyridinylmethyl)-2,3,6,7,8,9-hexahydroimidazo[1,2-a]pyrido[3,4-e]pyrimidin-5(1H)-one (Compound 23)

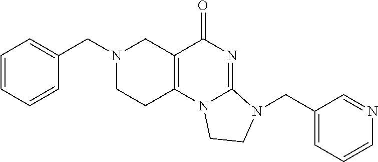 [SMILES: O=c1nc2n(c3c1CN(Cc1ccccc1)CC3)CCN2Cc1cccnc1]

Apart from the substitution of the key intermediate, the synthesis steps of the target product were the same as in Example 26, resulting in a white solid with a yield of 31%. ¹H NMR (400 MHZ, Chloroform-d) δ 8.57-8.50 (m, 2H), 7.75-7.68 (m, 1H), 7.36-7.20 (m, 6H), 4.63 (s, 2H), 3.92 (dd, J=9.7, 7.3 Hz, 2H), 3.69 (s, 2H), 3.52 (dd, J=9.7, 7.3 Hz, 2H), 3.42-3.37 (m, 2H), 2.73 -2.66 (m, 2H), 2.54-2.46 (m, 2H). ¹³C NMR (101 MHz, Chloroform-d) δ 170.98, 156.52, 149.58, 149.53, 142.52, 137.78, 136.46, 131.49, 129.10, 128.38, 127.31, 123.85, 111.27, 62.26, 50.14, 48.02, 45.62, 44.06, 42.32, 25.52.HRMS (ESI⁺): m/z calcd for C₂₂H₂₄N₅O [M+H]⁺374.1981, found 374.1980.

### Example 49: Preparation of 7-benzyl-3-(4-pyridinylmethyl)-2,3,6,7,8,9-hexahydroimidazo[1,2-a]pyrido[3,4-e]pyrimidin-5(1H)-one (Compound 24)

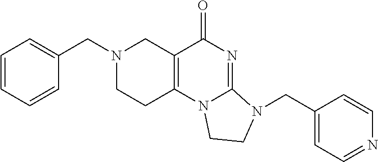 [SMILES: O=c1nc2n(c3c1CN(Cc1ccccc1)CC3)CCN2Cc1ccncc1]

Apart from the substitution of the key intermediate, the synthesis steps of the target product were the same as in Example 26, resulting in a white solid with a yield of 34%. ¹H NMR (400 MHZ, Chloroform-d) δ 8.51-8.45 (m, 2H), 7.29-7.22 (m, 4H), 7.21-7.17 (m, 1H), 7.17-7.11 (m, 2H), 4.55 (s, 2H), 3.87 (dd, J=9.7, 7.3 Hz, 2H), 3.62 (s, 2H), 3.46 (dd, J=9.7, 7.3 Hz, 2H), 3.36-3.30 (m, 2H), 2.63 (t, J=5.7 Hz, 2H), 2.49-2.41 (m, 2H). ¹³C NMR (101 MHZ, Chloroform-d) δ 169.86, 155.59, 149.25, 143.88, 141.43, 136.75, 128.09, 127.37, 126.32, 121.98, 110.43, 61.27, 49.14, 47.02, 46.20, 43.33, 41.34, 24.55.HRMS (ESI⁺): m/z calcd for C₂₂H₂₄N₅[M+H]⁺374.1981, found 374.1985.

### Example 50: Preparation of 7-benzyl-3-(2-furfuryl)-2,3,6,7,8,9-hexahydroimidazo[1,2-a]pyrido[3,4-e]pyrimidin-5(1H)-one (Compound 25)

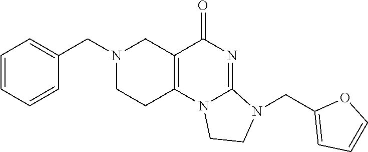 [SMILES: O=c1nc2n(c3c1CN(Cc1ccccc1)CC3)CCN2Cc1ccco1]

Apart from the substitution of the key intermediate, the synthesis steps of the target product were the same as in Example 26, resulting in a white solid with a yield of 30%. ¹H NMR (400 MHZ, DMSO-d₆) δ 7.61 (d, J=1.9 Hz, 1H), 7.37-7.19 (m, 5H), 6.45-6.27 (m, 2H), 4.47 (s, 2H), 3.98 (dd, J=9.6, 7.3 Hz, 2H), 3.61 (s, 2H), 3.54-3.42 (m, 2H), 3.01 (s, 2H), 2.67-2.58 (m, 2H), 2.57 -2.50 (m, 2H).¹³C NMR (101 MHz, DMSO-d₆) δ 169.98, 156.59, 150.31, 143.86, 143.47, 138.85, 129.23, 128.71, 127.50, 111.02, 109.52, 109.20, 61.98, 49.91, 48.87, 44.76, 42.36, 40.77, 24.97.HRMS (ESI⁺): m/z calcd for C₂₁H₂₃N₄O₂[M+H]⁺363.1821, found 363.1807.

### Example 51: Preparation of 7-benzyl-3-(3,4-dichlorobenzyl)-2,3,6,7,8,9-hexahydroimidazo[1,2-a]pyrido[3,4-e]pyrimidin-5(1H)-one (Compound 26)

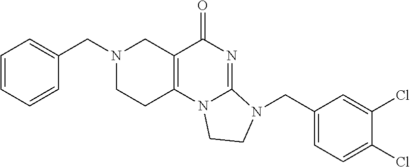 [SMILES: O=c1nc2n(c3c1CN(Cc1ccccc1)CC3)CCN2Cc1ccc(Cl)c(Cl)c1]

Apart from the substitution of the key intermediate, the synthesis steps of the target product were the same as in Example 26, resulting in a white solid with a yield of 38%. ¹H NMR (400 MHZ, DMSO-d₆) δ 7.61 (d, J=8.2 Hz, 1H), 7.57 (d, J=2.0 Hz, 1H), 7.36-7.22 (m, 6H), 4.47 (s, 2H), 4.01 (dd, J=9.7, 7.4 Hz, 2H), 3.62 (s, 2H), 3.51 (dd, J=9.7, 7.3 Hz, 2H), 3.01 (s, 2H), 2.68-2.60 (m, 2H), 2.59-2.51 (m, 2H). ¹³C NMR (101 MHz, DMSO-d₆) δ 170.06, 156.78, 143.95, 138.84, 138.57, 131.64, 131.22, 130.49, 130.22, 129.22, 128.72, 128.63, 127.51, 109.45, 62.00, 49.93, 48.89, 46.48, 44.77, 42.49, 25.04.HRMS (ESI⁺): m/z calcd for C₂₃H₂₃Cl2N₄O [M+H]⁺441.1249, found441.1243.

### Example 52: Preparation of 7-tert-butoxycarbonyl-3-(2-methylbenzyl)-2,3,6,7,8,9-hexahydroimidazo[1,2-a]pyrido[3,4-e]pyrimid in-5(1H)-one (Compound 27)

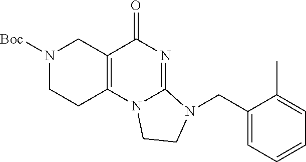 [SMILES: Cc1ccccc1CN1CCn2c1nc(=O)c1c2CCN(C(=O)OC(C)(C)C)C1]

Apart from the substitution of the key intermediate, the synthesis steps of the target product were the same as in Example 26, resulting in a white solid with a yield of 41%. ¹H NMR (400 MHZ, DMSO-d₆) δ 7.32-7.07 (m, 4H), 4.48 (s, 2H), 4.13-3.85 (m, 4H), 3.60-3.49 (m, 2H), 3.49-3.38 (m, 2H), 2.59-2.50 (m, 2H), 2.28 (s, 3H), 1.41 (s, 9H). ¹³C NMR (101 MHz, DMSO-d₆) δ 169.82, 156.81, 154.34, 143.99, 136.75, 134.73, 130.79, 128.46, 127.98, 126.41, 109.70, 79.63, 45.91, 44.79, 42.51, 28.54, 24.15, 19.14.HRMS (ESI⁺): m/z calcd for C₂₂H₂₉N₄O₃[M+H]⁺397.2240, found 397.2241.

### Example 53: Preparation of 7-ethyl-3-(2-methylbenzyl)-2,3,6,7,8,9-hexahydroimidazo[1,2-a]pyrido[3,4-e]pyrimidin-5(1H)-one (Compound 28)

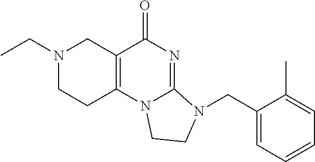 [SMILES: CCN1CCc2c(c(=O)nc3n2CCN3Cc2ccccc2C)C1]

To a solution of compound 27 in dichloromethane was added trifluoroacetic acid and reacted for 1-3 hours. The reaction mixture was concentrated under reduced pressure to remove trifluoroacetic acid and dichloromethane to afford the deprotected product intermediate 2. The volume ratio of dichloromethane to trifluoroacetic acid was 2:1. Intermediate 2 was dissolved in acetonitrile, followed by the sequential addition of anhydrous potassium carbonate (3 eq) and bromoethane (1.5 eq). The reaction mixture was heated to 40° C. overnight. After filtration, the filtrate was concentrated under reduced pressure and went through silica gel column chromatography using methanol:dichloromethane gradient (0:100 to 10:100) to obtain the desired product. Yield 55%, white solid. ¹H NMR (400 MHZ, DMSO-d₆) δ 7.25-7.14 (m, 4H), 4.47 (s, 2H), 4.01 (dd, J=9.7, 7.5 Hz, 2H), 3.45 (d, J=17.1 Hz, 2H), 3.04 (s, 2H), 2.63-2.56 (m, 2H), 2.55-2.51 (m, 2H), 2.49 -2.43 (m, 2H), 2.28 (s, 3H), 1.05 (t, J=7.2 Hz, 3H).¹³C NMR (101 MHz, DMSO-d₆) δ 170.16, 156.73, 143.88, 136.73, 134.84, 130.77, 128.46, 127.94, 126.40, 109.29, 51.65, 49.68, 48.77, 45.97, 44.82, 42.38, 25.03, 19.14, 12.81.HRMS (ESI⁺): m/z calcd for C₁₉H₂₅N₄O [M+H]⁺325.2028, found 325.2025.

### Example 54: Preparation of 7-isobutyl-3-(2-methylbenzyl)-2,3,6,7,8,9-hexahydroimidazo[1,2-a]pyrido[3,4-e]pyrimidin-5(1H)-o ne (Compound 29)

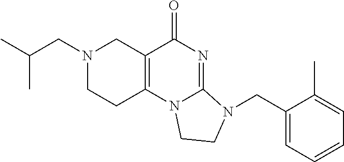 [SMILES: Cc1ccccc1CN1CCn2c1nc(=O)c1c2CCN(CC(C)C)C1]

Apart from the substitution of the key intermediate, the synthesis steps of the target product were the same as in Example 53, resulting in a white solid with a yield of 33%. ¹H NMR (400 MHZ, DMSO-d₆) δ 7.30-7.10 (m, 4H), 4.47 (s, 2H), 4.01 (dd, J=9.7, 7.4 Hz, 2H), 3.44 (dd, J=9.7, 7.5 Hz, 2H), 3.01 (s, 2H), 2.60-2.51 (m, 4H), 2.28 (s, 3H), 2.18 (d, J=7.4 Hz, 2H), 1.81 (dp, J=13.0, 6.5 Hz, 1H), 0.86 (d, J=6.5 Hz, 6H). ¹³C NMR (101 MHz, Chloroform-d) δ 174.95, 161.47, 148.69, 141.49, 139.59, 135.52, 133.20, 132.69, 131.15, 114.11, 70.99, 55.33, 54.21, 50.72, 49.58, 47.12, 30.52, 29.82, 25.91, 23.89.HRMS (ESI⁺): m/z calcd for C₂₁H₂₉N₄O [M+H]⁺353.2341, found 353.2339.

### Example 55: Preparation of 7-cyclopropyl-3-(2-methylbenzyl)-2,3,6,7,8,9-hexahydroimidazo[1,2-a]pyrido[3,4-e]pyrimidin-5(1H)-one (Compound 30)

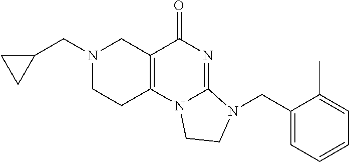 [SMILES: Cc1ccccc1CN1CCn2c1nc(=O)c1c2CCN(CC2CC2)C1]

Apart from the substitution of the key intermediate, the synthesis steps of the target product were the same as in Example 53, resulting in a white solid with a yield of 51%. ¹H NMR (400 MHZ, DMSO-d₆) δ 7.30-7.13 (m, 4H), 4.48 (s, 2H), 4.02 (dd, J=9.7, 7.4 Hz, 2H), 3.46 (dd, J=9.7, 7.4 Hz, 2H), 3.15 (s, 2H), 2.72-2.61 (m, 2H), 2.60-2.52 (m, 2H), 2.35 (d, J=6.6 Hz, 2H), 2.29 (s, 3H), 0.97-0.78 (m, 1H), 0.57-0.43 (m, 2H), 0.18-0.05 (m, 2H).¹³C NMR (101 MHz, DMSO-d₆) 8 170.15, 156.71, 143.84, 136.73, 134.84, 130.77, 128.44, 127.94, 126.40, 109.36, 62.64, 49.96, 48.95, 45.97, 44.81, 42.38, 24.94, 19.15, 19.12, 9.04, 4.23.HRMS (ESI⁺): m/z calcd for C₂₁H₂₇N₄O [M+H]⁺351.2185, found 351.2182.

### Example 56: Preparation of 7-cyclohexylmethyl-3-(2-methylbenzyl)-2,3,6,7,8,9-hexahydroimidazo[1,2-a]pyrido[3,4-e]pyrimidin-5(1H)-one (Compound 31)

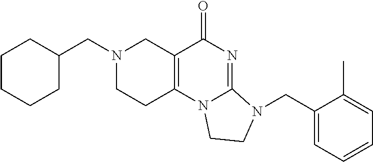 [SMILES: Cc1ccccc1CN1CCn2c1nc(=O)c1c2CCN(CC2CCCCC2)C1]

Apart from the substitution of the key intermediate, the synthesis steps of the target product were the same as in Example 53, resulting in a white solid with a yield of 49%. ¹H NMR (400 MHZ, Chloroform-d) δ 7.25-7.11 (m, 4H), 4.63 (s, 2H), 3.98-3.83 (m, 2H), 3.51-3.40 (m, 2H), 3.33 (t, J=2.0 Hz, 2H), 2.69 (t, J=5.7 Hz, 2H), 2.55 (dq, J=5.8, 3.8, 2.9 Hz, 2H), 2.37-2.27 (m, 5H), 1.30-1.15 (m, 3H), 0.97-0.81 (m, 2H).¹³C NMR (101 MHz, Chloroform-d) δ 171.23, 156.44, 142.18, 137.20, 133.53, 130.72, 129.24, 128.14, 126.13, 111.18, 65.31, 50.17, 49.58, 46.57, 44.19, 42.27, 35.11, 31.83, 26.74, 26.05, 25.44, 19.24.HRMS (ESI⁺): m/z calcd for C₂₃H₃₃N₄O [M+H] 393.2654, found 393.2651.

### Example 57: Preparation of 3-(2-methylbenzyl)-7-(3-methylbenzyl)-2,3,6,7,8,9-hexahydroimidazo[1,2-a]pyrido[3,4-e]pyrimidin-5(1H)-one (Compound 32)

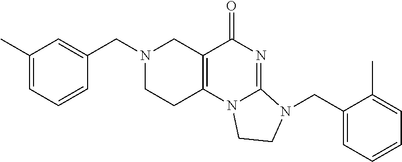 [SMILES: Cc1cccc(CN2CCc3c(c(=O)nc4n3CCN4Cc3ccccc3C)C2)c1]

Apart from the substitution of the key intermediate, the synthesis steps of the target product were the same as in Example 53, resulting in a white solid with a yield of 61%. ¹H NMR (400 MHZ, DMSO-d₆) δ 7.17 (d, J=26.2 Hz, 8H), 4.47 (s, 2H), 4.18-3.85 (m, 2H), 3.58 (s, 2H), 3.53-3.41 (m, 2H), 3.00 (s, 2H), 2.78-2.60 (m, 2H), 2.61-2.52 (m, 2H), 2.30 (s, 3H), 2.28 (s, 3H).¹³C NMR (101 MHz, Chloroform-d) δ 171.20, 156.40, 142.45, 137.98, 137.68, 137.11, 133.50, 130.69, 129.85, 129.16, 128.24, 128.11, 128.06, 126.22, 126.13, 111.05, 62.29, 50.23, 48.01, 46.47, 44.16, 42.28, 25.50, 21.38, 19.21.HRMS (ESI⁺): m/z calcd for C₂₅H₂₉N₄O [M+H]⁺401.2341, found 401.2341.

### Example 58: Preparation of 3-(2-methylbenzyl)-7-(4-methylbenzyl)-2,3,6,7,8,9-hexahydroimidazo[1,2-a]pyrido[3,4-e]pyrimidin-5(1H)-one (Compound 33)

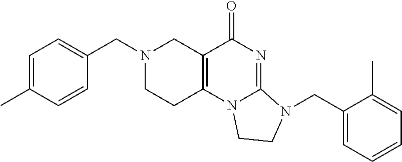 [SMILES: Cc1ccc(CN2CCc3c(c(=O)nc4n3CCN4Cc3ccccc3C)C2)cc1]

Apart from the substitution of the key intermediate, the synthesis steps of the target product were the same as in Example 53, resulting in a white solid with a yield of 63%. ¹H NMR (400 MHZ, DMSO-d₆) δ 7.66-6.92 (m, 8H), 4.46 (s, 2H), 4.16-3.88 (m, 2H), 3.61-3.53 (m, 2H), 3.49-3.42 (m, 2H), 3.11-2.92 (m, 2H), 2.50 (s, 3H), 2.28 (s, 3H).¹³C NMR (101 MHZ, DMSO-d₆) δ 171.11, 156.41, 142.31, 137.14, 136.88, 134.70, 133.54, 130.70, 129.20, 129.11, 129.05, 128.10, 126.12, 111.13, 61.99, 50.23, 47.89, 46.49, 44.15, 42.27, 25.54, 21.11, 19.23.HRMS (ESI⁺): m/z calcd for C₂₅H₂₉N₄O [M+H]⁺401.2341, found 401.2342.

### Example 59: Preparation of 2-((3-(2-methylbenzyl)-5-oxo-2,3,6,7,8,9-hexahydroimidazo[1,2-a]pyrido[3,4-e]pyrimidin-7(6H)-yl) methyl)benzonitrile (Compound 34)

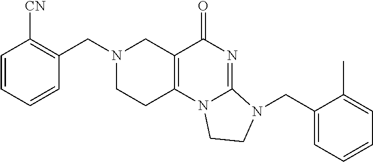 [SMILES: Cc1ccccc1CN1CCn2c1nc(=O)c1c2CCN(Cc2ccccc2C#N)C1]

Apart from the substitution of the key intermediate, the synthesis steps of the target product were the same as in Example 53, resulting in a white solid with a yield of 75%. ¹H NMR (400 MHZ, DMSO-d₆) δ 7.88-7.79 (m, 1H), 7.74-7.65 (m, 1H), 7.63-7.56 (m, 1H), 7.53-7.45 (m, 1H), 7.26-7.13 (m, 4H), 4.47 (s, 2H), 4.02 (dd, J=9.7, 7.4 Hz, 2H), 3.81 (s, 2H), 3.45 (dd, J=9.7, 7.3 Hz, 2H), 3.08 (s, 2H), 2.80-2.66 (m, 2H), 2.62-2.53 (m, 2H), 2.28 (s, 3H).¹³C NMR (101 MHZ, Chloroform-d) δ 171.01, 156.46, 137.16, 133.48, 133.15, 132.79, 130.72, 130.20, 129.21, 128.14, 127.90, 126.14, 117.90, 113.01, 110.72, 60.19, 49.48, 49.05, 46.54, 44.21, 42.30, 25.38, 19.24.HRMS (ESI⁺): m/z calcd for C₂₅H₂₆N₅O₄[M+H]⁺412.2137, found 412.2140.

### Example 60: Preparation of 3-((3-(2-methylbenzyl)-5-oxo-2,3,6,7,8,9-hexahydroimidazo[1,2-a]pyrido[3,4-e]pyrimidin-7(6H)-yl) methyl)benzonitrile (Compound 35)

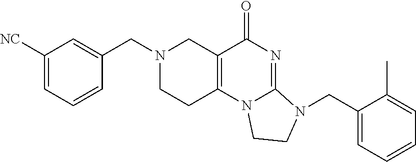 [SMILES: Cc1ccccc1CN1CCn2c1nc(=O)c1c2CCN(Cc2cccc(C#N)c2)C1]

Apart from the substitution of the key intermediate, the synthesis steps of the target product were the same as in Example 53, resulting in a white solid with a yield of 85%. ¹H NMR (400 MHZ, DMSO-d₆) δ 7.81-7.72 (m, 2H), 7.68 (dt, J=7.8, 1.5 Hz, 1H), 7.56 (t, J=7.7 Hz, 1H), 7.27-7.14 (m, 4H), 4.47 (s, 2H), 4.02 (dd, J=9.7, 7.4 Hz, 2H), 3.70 (s, 2H), 3.46 (dd, J=9.7, 7.4 Hz, 2H), 3.05 (s, 2H), 2.71-2.61 (m, 2H), 2.61-2.53 (m, 2H), 2.28 (s, 3H).¹³C NMR (101 MHZ, DMSO-d₆) 8 170.09, 156.73, 143.90, 140.82, 136.73, 134.80, 134.08, 132.52, 131.40, 130.77, 130.02, 128.45, 127.95, 126.40, 119.35, 111.75, 109.07, 60.76, 49.91, 48.79, 45.95, 44.82, 42.40, 25.00, 19.13.HRMS (ESI⁺): m/z calcd for C₂₅H₂₆N₅[M+H]⁺412.2137, found 412.2142.

### Example 61: Preparation of 7-(3,4-difluorobenzyl)-3-(2-methylbenzyl)-2,3,6,7,8,9-hexahydroimidazo[1,2-a]pyrido[3,4-e]pyrimi din-5(1H)-one (Compound 36)

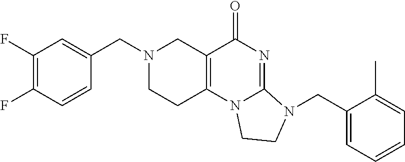 [SMILES: Cc1ccccc1CN1CCn2c1nc(=O)c1c2CCN(Cc2ccc(F)c(F)c2)C1]

Apart from the substitution of the key intermediate, the synthesis steps of the target product were the same as in Example 53, resulting in a white solid with a yield of 73%. ¹H NMR (400 MHZ, DMSO-d₆) δ 7.44-7.32 (m, 2H), 7.20 (qd, J=7.8, 7.3, 2.9 Hz, 5H), 4.48 (s, 2H), 4.03 (dd, J=9.7, 7.4 Hz, 2H), 3.63 (s, 2H), 3.46 (dd, J=9.8, 7.3 Hz, 2H), 3.04 (s, 2H), 2.69-2.62 (m, 2H), 2.60 -2.54 (m, 2H), 2.28 (s, 3H).¹³C NMR (101 MHz, Chloroform-d) δ 171.04, 156.47, 150.36 (dd, J=247.8, 13.4 Hz), 149.60 (dd, J=247.2, 12.5 Hz), 142.17, 137.14, 135.20, 133.47, 130.73, 129.21, 128.16, 126.15, 124.69 (dd, J=6.33, 3.57 Hz), 117.28 (dd, J=55.8, 17.2 Hz), 117.19 (d, J=55.6 Hz), 110.93, 61.15, 50.07, 48.17, 46.53, 44.19, 42.31, 25.54, 19.23.HRMS (ESI⁺): m/z calcd for C₂₄H₂₅F₂N₄O [M+H]⁺423.1996, found 423.1999.

### Example 62: Preparation of 7-(2, 4-difluorobenzyl)-3-(2-methylbenzyl)-2,3,6,7,8,9-hexahydroimidazo[1,2-a]pyrido[3,4-e]pyrimi din-5(1H)-one (Compound 37)

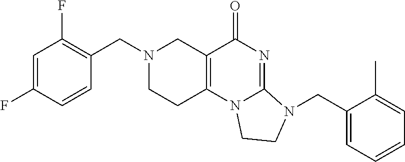 [SMILES: Cc1ccccc1CN1CCn2c1nc(=O)c1c2CCN(Cc2ccc(F)cc2F)C1]

Apart from the substitution of the key intermediate, the synthesis steps of the target product were the same as in Example 53, resulting in a white solid with a yield of 71%. ¹H NMR (400 MHZ, DMSO-d₆) δ 7.54-7.42 (m, 1H), 7.31-7.13 (m, 5H), 7.12-7.04 (m, 1H), 4.47 (s, 2H), 4.02 (dd, J=9.7, 7.4 Hz, 2H), 3.66 (s, 2H), 3.45 (dd, J=9.8, 7.4 Hz, 2H), 3.05 (s, 2H), 2.74-2.62 (m, 2H), 2.60-2.52 (m, 2H), 2.28 (s, 3H). ¹³C NMR (101 MHz, Chloroform-d) δ 171.04, 162.26 (dd, J=247.9 Hz, 12.0 Hz), 161.34 (dd, J=237.2 Hz, 11.7 Hz), 156.42, 142.25, 137.11, 133.47, 132.30 (dd, J=9.6, 5.9 Hz), 130.71, 129.18, 128.13, 126.14, 120.49 (dd, J=14.6, 3.6 Hz), 111.19 (dd, J=21.2, 3.7 Hz), 110.80, 103.73 (t, J=25.7 Hz), 54.23, 49.79, 48.08, 46.48, 44.15, 42.28, 25.45, 19.22.HRMS (ESI⁺): m/z calcd for C₂₄H₂₅F₂N₄O [M+H]⁺423.1996, found 423.1998.

### Example 63: Preparation of 7-(4-fluorobenzyl)-3-(2-methylbenzyl)-2,3,6,7,8,9-hexahydroimidazo[1,2-a]pyrido[3,4-e]pyrimidin-5(1H)-one (Compound 38)

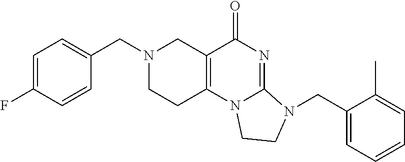 [SMILES: Cc1ccccc1CN1CCn2c1nc(=O)c1c2CCN(Cc2ccc(F)cc2)C1]

Apart from the substitution of the key intermediate, the synthesis steps of the target product were the same as in Example 53, resulting in a white solid with a yield of 78%. ¹H NMR (400 MHZ, Chloroform-d) δ 7.33-7.28 (m, 2H), 7.25-7.12 (m, 4H), 7.03-6.95 (m, 2H), 4.63 (s, 2H), 3.96 -3.85 (m, 2H), 3.66 (s, 2H), 3.50-3.35 (m, 4H), 2.76-2.62 (m, 2H), 2.55-2.46 (m, 2H), 2.32 (s, 3H).¹³C NMR (101 MHZ, Chloroform-d) δ 171.11, 163.33, 160.89, 156.44, 142.30, 137.12, 133.64 (d, J=3.1 Hz), 133.50, 130.72, 130.62, 130.55, 129.19, 128.14, 126.14, 115.17 (d, J=20.9 Hz), 111.01, 61.48, 50.12, 48.03, 46.49, 44.18, 42.30, 25.54, 19.23.HRMS (ESI⁺): m/z calcd for C₂₄H₂₆FN₄O [M+H]⁺405.2091, found 405.2096.

### Example 64: Preparation of 7-benzoyl-3-(2-methylbenzyl)-2,3,6,7,8,9-hexahydroimidazo[1,2-a]pyrido[3,4-e]pyrimidin-5(1H)-one (Compound 39)

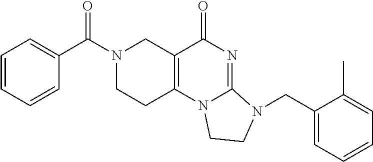 [SMILES: Cc1ccccc1CN1CCn2c1nc(=O)c1c2CCN(C(=O)c2ccccc2)C1]

Apart from the substitution of the key intermediate, the synthesis steps of the target product were the same as in Example 53, resulting in a white solid with a yield of 75%. ¹H NMR (400 MHZ, DMSO-d₆) δ 7.58-7.36 (m, 5H), 7.21 (d, J=3.6 Hz, 4H), 4.47 (s, 2H), 4.26 (s, 1H), 4.13-3.93 (m, 3H), 3.85 (s, 1H), 3.57-3.43 (m, 3H), 2.71-2.55 (m, 2H), 2.28 (s, 3H). ¹³C NMR (101 MHZ, Chloroform-d) δ 171.19, 170.37, 156.51, 142.78, 137.09, 134.99, 133.16, 130.78, 130.46, 129.18, 128.67, 128.27, 127.48, 126.20, 109.81, 46.49, 45.38, 44.21, 42.41, 38.45, 24.10, 19.24.HRMS (ESI⁺): m/z calcd for C₂₄H₂₅N₄O₂[M+H]⁺401.1978, found 401.1976.

### Example 65: Preparation of 3-(2-methylbenzyl)-7-(2-phenylacetyl)-2,3,6,7,8,9-hexahydroimidazo[1,2-a]pyrido[3,4-e]pyrimidin-5(1H)-one (Compound 40)

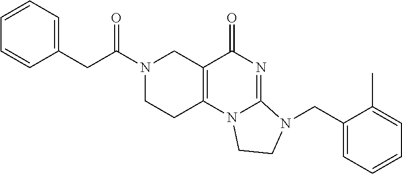 [SMILES: Cc1ccccc1CN1CCn2c1nc(=O)c1c2CCN(C(=O)Cc2ccccc2)C1]

Apart from the substitution of the key intermediate, the synthesis steps of the target product were the same as in Example 53, resulting in a white solid with a yield of 64%. ¹H NMR (400 MHZ, DMSO-d₆) δ 7.32-7.17 (m, 9H), 4.48 (s, 2H), 4.23-4.09 (m, 2H), 4.08-3.93 (m, 2H), 3.84-3.75 (m, 2H), 3.73-3.65 (m, 2H), 3.49-3.40 (m, 2H), 2.56-2.50 (m, 2H), 2.28 (s, 3H).¹³C NMR (101 MHz, Chloroform-d) δ 170.64, 170.46, 156.53, 142.86, 137.10, 134.63, 133.16, 130.79, 129.20, 129.11, 128.63, 128.28, 126.81, 126.20, 109.29, 46.49, 44.16, 43.13, 42.35, 40.60, 37.56, 24.20, 19.25.HRMS (ESI⁺): m/z calcd for C₂₅H₂₇N₄O₂[M+H]⁺415.2134, found 415.2130.

### Example 66: Preparation of 3-(2-methylbenzyl)-7-(4-phenylbutyl)-2,3,6,7,8,9-hexahydroimidazo[1,2-a]pyrido[3,4-e]pyrimidin-5 (1H)-one (Compound 41)

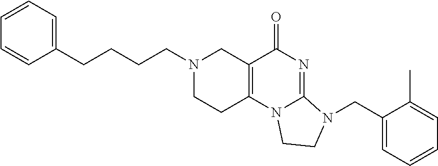 [SMILES: Cc1ccccc1CN1CCn2c1nc(=O)c1c2CCN(CCCCc2ccccc2)C1]

Apart from the substitution of the key intermediate, the synthesis steps of the target product were the same as in Example 53, resulting in a white solid with a yield of 65%. ¹H NMR (400 MHZ, DMSO-d₆) δ 7.31-7.13 (m, 9H), 4.47 (s, 2H), 4.01 (dd, J=9.7, 7.4 Hz, 2H), 3.45 (dd, J=9.7, 7.4 Hz, 2H), 3.03 (s, 2H), 2.65-2.55 (m, 4H), 2.54-2.51 (m, 2H), 2.45 (t, J=7.1 Hz, 2H), 2.28 (s, 3H), 1.65-1.54 (m, 2H), 1.54-1.44 (m, 2H).¹³C NMR (101 MHZ, DMSO-d₆) δ 170.20, 156.71, 143.91, 142.72, 136.73, 134.83, 130.77, 128.74, 128.71, 128.46, 127.94, 126.40, 126.10, 109.28, 57.55, 50.15, 49.12, 45.96, 44.81, 42.37, 35.47, 29.26, 26.71, 25.03, 19.14. HRMS (ESI⁺): m/z calcd for C₂₇H₃₃N₄O [M+H]⁺429.2654, found 429.2656.

### Example 67: Preparation of 3-(2-methylbenzyl)-7-phenethyl-2,3,6,7,8,9-hexahydroimidazo[1,2-a]pyrido[3,4-e]pyrimidin-5(1H)-one (Compound 42)

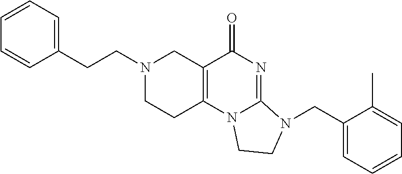 [SMILES: Cc1ccccc1CN1CCn2c1nc(=O)c1c2CCN(CCc2ccccc2)C1]

Apart from the substitution of the key intermediate, the synthesis steps of the target product were the same as in Example 53, resulting in a white solid with a yield of 60%. ¹H NMR (400 MHZ, DMSO-d₆) δ 7.34-7.13 (m, 9H), 4.47 (s, 2H), 4.00 (dd, J=9.7, 7.5 Hz, 2H), 3.50-3.40 (m, 2H), 3.14 (s, 2H), 2.78 (dd, J=9.0, 6.0 Hz, 2H), 2.73-2.63 (m, 4H), 2.54-2.50 (m, 2H), 2.28 (s, 3H).¹³C NMR (101 MHz, DMSO-d₆) δ 170.18, 156.72, 143.89, 140.84, 136.73, 134.83, 130.77, 129.14, 128.70, 128.46, 127.94, 126.40, 126.30, 109.29, 59.50, 50.06, 48.96, 45.97, 44.83, 42.38, 33.45, 25.02, 19.14.HRMS (ESI⁺): m/z calcd for C₂₅H₂₉N₄O [M+H]+, found 401.2341.

### Example 68: Preparation of 3-(2-methylbenzyl)-7-(pyridin-3-methyl)-2,3,6,7,8,9-hexahydroimidazo[1,2-a]pyrido[3,4-e]pyrimidi n-5(1H)-one (Compound 43)

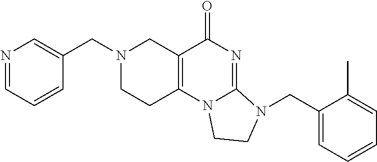 [SMILES: Cc1ccccc1CN1CCn2c1nc(=O)c1c2CCN(Cc2cccnc2)C1]

Apart from the substitution of the key intermediate, the synthesis steps of the target product were the same as in Example 53, resulting in a white solid with a yield of 78%. ¹H NMR (400 MHZ, DMSO-d₆) δ 8.56-8.45 (m, 2H), 7.73 (dt, J=7.8, 2.0 Hz, 1H), 7.37 (dd, J=7.8, 4.8 Hz, 1H), 7.26-7.15 (m, J=5.0, 4.4 Hz, 4H), 4.47 (s, 2H), 4.02 (dd, J=9.7, 7.4 Hz, 2H), 3.67 (s, 2H), 3.45 (dd, J =9.8, 7.5 Hz, 2H), 3.04 (d, J=2.1 Hz, 2H), 2.71-2.63 (m, 2H), 2.60-2.52 (m, 2H), 2.28 (s, 3H). ¹³C NMR (101 MHz, DMSO-d₆) δ 170.12, 156.72, 150.47, 148.89, 143.92, 136.97, 136.73, 134.79, 134.26, 130.77, 128.44, 127.95, 126.39, 123.96, 109.06, 59.06, 49.87, 48.80, 45.94, 44.81, 42.41, 24.97, 19.13.HRMS (ESI⁺): m/z calcd for C₂₃H₂₆N₅[M+H]⁺388.2137, found 388.2135.

### Example 69: Preparation of 3-(2-methylbenzyl)-7-(pyridin-4-methyl)-2,3,6,7,8,9-hexahydroimidazo[1,2-a]pyrido[3,4-e]pyrimidi n-5(1H)-one (Compound 44)

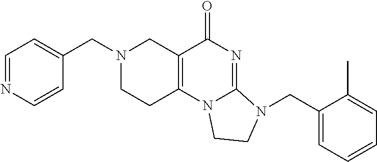 [SMILES: Cc1ccccc1CN1CCn2c1nc(=O)c1c2CCN(Cc2ccncc2)C1]

Apart from the substitution of the key intermediate, the synthesis steps of the target product were the same as in Example 53, resulting in a white solid with a yield of 80%. ¹H NMR (400 MHZ, DMSO-d₆) δ 8.58-8.45 (m, 2H), 7.40-7.31 (m, 2H), 7.27-7.15 (m, 4H), 4.47 (s, 2H), 4.03 (dd, J=9.8, 7.4 Hz, 2H), 3.68 (s, 2H), 3.46 (dd, J=9.7, 7.4 Hz, 2H), 3.06 (s, 2H), 2.71-2.63 (m, 2H), 2.61-2.53 (m, 2H), 2.28 (s, 3H). ¹³C NMR (101 MHz, DMSO-d₆) δ 170.07, 156.74, 150.09, 148.09, 143.85, 136.73, 134.80, 130.78, 128.46, 127.95, 126.40, 124.15, 109.06, 60.56, 50.06, 48.95, 45.95, 44.83, 42.41, 24.97, 19.13.HRMS (ESI⁺): m/z calcd for C₂₃H₂₆N₅[M+H]⁺388.2137, found 388.2141.

### Example 70: Preparation of 3-(2-methylbenzyl)-7-(1, 3, 4-thiadiazol-2-yl)-2,3,6,7,8,9-hexahydroimidazo[1,2-a]pyrido[3,4-e]pyri midin-5(1H)-one (Compound 45)

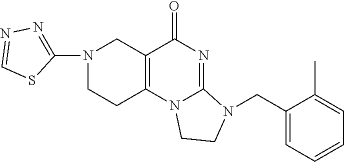 [SMILES: Cc1ccccc1CN1CCn2c1nc(=O)c1c2CCN(c2nncs2)C1]

To a solution of intermediate 2 in dry 1, 4-dioxanewas added aryl halide (1.5 eq), cesium carbonate (3 eq), and a catalytic amount of Pd(dppf)Cl2 under a nitrogen atmosphere. The reaction was refluxed for 4 hours, and then concentrated under reduced pressure to remove solvent. The residue was purified by silica gel column chromatography to obtain a white solid with a yield of 52%. ¹H NMR (400 MHZ, DMSO-d₆) δ 8.84 (s, 1H), 7.26-7.13 (m, 4H), 4.49 (s, 2H), 4.13-4.00 (m, 4H), 3.85-3.76 (m, 2H), 3.48 (dd, J=9.7, 7.5 Hz, 2H), 2.77-2.66 (m, 2H), 2.28 (s, 3H). ¹³C NMR (101 MHz, DMSO-d₆) δ 172.01, 169.69, 156.86, 144.48, 143.86, 136.74, 134.66, 130.79, 128.46, 128.00, 126.42, 107.15, 47.77, 45.91, 45.48, 44.81, 42.56, 23.34, 19.15.HRMS (ESI⁺): m/z calcd for C₁₉H₂₁N₆OS [M+H] 381.1498, found 381.1493.

### Example 71: Preparation of 1-(2-methylbenzyl) tetrahydropyrimidin-2(1H)-imine hydrobromide (Intermediate 4)

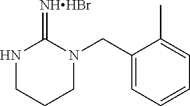

The synthesis steps of this intermediate were the same as in Example 1, resulting in a white solid with a yield of 80%. ¹H NMR (400 MHZ, DMSO-d₆) δ 7.88 (d, J=4.9 Hz, 1H), 7.07-7.00 (m, 1H), 4.60 (s, 2H), 3.32-3.23 (m, 4H), 2.25 (s, 3H), 1.96-1.89 (m, 2H). ¹³C NMR (101 MHZ, DMSO-d₆) δ 154.16, 136.43, 133.48, 130.91, 127.81, 126.64, 125.50, 50.89, 46.04, 38.29, 20.73, 19.04.

### Example 72: Preparation of 3-Benzyl-7-(2-methylbenzyl)-1, 2, 3, 4, 7, 8, 9, 10-octahydro-5H-pyrido[3,4-e]pyrimido[1,2-a] pyrimidin-5-one (Compound 46)

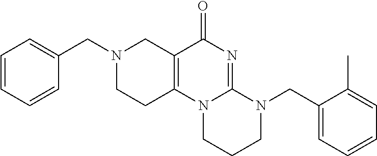 [SMILES: Cc1ccccc1CN1CCCn2c1nc(=O)c1c2CCN(Cc2ccccc2)C1]

Apart from the substitution of the key intermediate, the synthesis steps of the target product were the same as in Example 26, resulting in a white solid with a yield of 65%.¹H NMR (400 MHZ, Chloroform-d) δ 7.42-7.27 (m, 4H), 7.25-7.08 (m, 5H), 4.88 (s, 2H), 3.99 (dd, J=6.9, 5.0 Hz, 2H), 3.70 (s, 2H), 3.42 (s, 2H), 3.23 (t, J=6.0 Hz, 2H), 2.66 (t, J=5.8 Hz, 2H), 2.54 (t, J=5.8 Hz, 2H), 2.28 (s, 3H), 2.04-1.93 (m, 2H).¹³C NMR (101 MHz, Chloroform-d) δ 161.33, 158.72, 150.67, 138.30, 136.51, 134.80, 130.54, 129.14, 128.26, 127.74, 127.39, 127.05, 126.06, 106.79, 62.64, 50.24, 50.00, 49.55, 44.74, 39.38, 32.10, 20.40, 19.24.HRMS (ESI⁺): m/z calcd for C₂₅H₂₉N₄O [M+H]⁺401.2341, found 401.2355.

### Example 73: Preparation of 7-benzyl-9, 9-difluoro-4-(4-methoxybenzyl)-2, 4, 6, 7, 8, 9-hexahydroimidazo[1,2-a] pyrido[3,4-e]pyrimidin-5(1H)-one (Intermediate 5a)

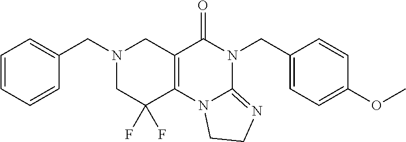 [SMILES: COc1ccc(CN2C(=O)C3=C(N4CCN=C24)C(F)(F)CN(Cc2ccccc2)C3)cc1]

To a solution of ingredient 5a: (methyl 1-benzyl-5, 5-difluoro-4-oxopiperidin-3-carboxylate hydrochloride) (2 g, 6.27 mmol), and ingredient 6:

(N-(4-methoxybenzyl)-4, 5-dihydro-1H-imidazol-2-amine) (1.29 g, 6.27 mmol) in anhydrous methanol (100 mL) was added sodium methoxide (846 mg, 15.6 mmol), and the mixture was heated and refluxed for 6 hours. TLC showed that the reaction was completed. The reaction solution was concentrated under reduced pressure to remove solven, and the residue was suspended in water, and the mixture was extracted with dichloromethane three times. The combined organic phases were washed with water three times, and saturated salt water once and dried over anhydrous sodium sulfate. After filtration, the filtrate was concentrated under reduced pressure. The residue was then suspended in ethyl acetate and beat, and filtered to obtain a white solid (2.06 g, 4.7 mmol) with a yield of 70%.¹H NMR (400 MHZ, DMSO-d₆) δ 7.45-7.18 (m, 7H), 6.84 (d, J=8.7 Hz, 2H), 4.87 (s, 2H), 4.03 (t, J=9.0 Hz, 2H), 3.83-3.75 (m, 2H), 3.73 (s, 2H), 3.71 (s, 3H), 3.20 (d, J=4.6 Hz, 2H), 3.09 (t, J=12.3 Hz, 2H). HRMS (ESI): calcd. for [M+H]⁺439.1946, found 439.1939.

### Example 74: Preparation of 3-((9, 9-difluoro-4-(4-methoxybenzyl)-5-oxo-1, 2, 4, 5, 8, 9-hexahydroimidazo[1,2-a] pyrido [3,4-e]pyrimidin-7(6H)-yl)methyl)benzonitrile (Intermediate 5b)

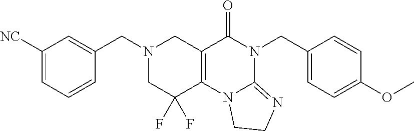 [SMILES: COc1ccc(CN2C(=O)C3=C(N4CCN=C24)C(F)(F)CN(Cc2cccc(C#N)c2)C3)cc1]

To a solution of ingredient 5b (methyl 1-(3-cyanophenyl)-5, 5-difluoro-4-oxypiperidin-3-carboxylate ester hydrochloride) (3.44 g, 10 mmol), , and ingredient 6 (N-(4-methoxybenzyl)-4, 5-dihydro-1H-imidazol-2-amine) (2.05 g, 10 mmol) in anhydrous methanol (250 mL) was added sodium methoxide (1.35 g, 25 mmol), and the mixture was heated and refluxed for 6 hours. TLC showed that the reaction was completed. The reaction solution was concentrated under reduced pressure to remove solvent. The residue was suspended in water, and extracted three times with dichloromethane. The combined organic phases were washed with water three times and with saturated salt water once and then dried over anhydrous sodium sulfate. After filtration, the filtrate was concentrated under reduced pressure and then suspended in ethyl acetate to beat, and then filtered to afford a white solid (2.78 g, 6.9 mmol) with a yield of 69%. ¹H NMR (400 MHZ, DMSO-d₆) δ 7.83-7.51 (m, 4H), 7.31 (d, J=8.7 Hz, 2H), 6.85 (d, J=8.7 Hz, 2H), 4.88 (s, 2H), 4.04 (t, J=9.0 Hz, 2H), 3.86-3.76 (m, 4H), 3.72 (s, 3H), 3.30-3.20 (m, 2H), 3.13 (t, J=12.2 Hz, 2H).HRMS (ESI): calcd. for [M+H]⁺464.1898, found 464.1895.

### Example 75: Preparation of 7-Benzyl-9, 9-difluoro-2, 4, 6, 7, 8, 9-hexahydroimidazo [1,2-a]pyrido[3,4-e] pyrimidin-5(1H)-one (Intermediate 6a)

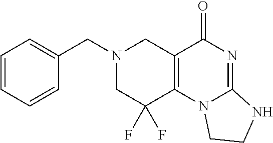 [SMILES: O=c1nc2n(c3c1CN(Cc1ccccc1)CC3(F)F)CCN2]

To a solution of Intermediate 5a: 7-Benzyl-9, 9-difluoro-4-(4-methoxybenzyl)-2, 4, 6, 7, 8, 9-hexahydroimidazo[1,2-a]pyrido[3,4-e]pyrimidin-5(1H)-one (2 g, 4.5 mmol) in dry dichloromethane (50 mL) was added dry aluminum trichloride (1.82 g, 13.7 mmol) under nitrogen protection and stirred overnight at room temperature. TLC showed that the reaction was completed.

The reaction solution was quenched with water and adjusted pH to strong alkaline with sodium hydroxide solution The mixture was partitioned. The aqueous layer was extracted three times with methanol:chloroform (1:9), and the combined organic phases were washed three times with water, once with saturated salt water, and dried over anhydrous sodium sulfate. After filtration, the filtrate was concentrated under reduced pressure and the residue was suspended in ethyl acetate to beat. A white solid (572 mg, 1.8 mmol) was obtained after filtrationwith a yield of 40%. ¹H NMR (400 MHZ, DMSO-d₆) δ 8.01 (s, 1H), 7.65-6.97 (m, 5H), 4.17 (s, 2H), 3.74 (s, 2H), 3.61 (s, 2H), 3.24-2.91 (m, 4H). HRMS (ESI): calcd. for [M+H]⁺319.1370, found 319.1367.

### Example 76: Preparation of 3-((9, 9-difluoro-5-oxo-1, 2, 4, 5, 8, 9-hexahydroimidazo [1,2-a]pyrido[3,4-e]pyrimidin-7(6H)-yl)methyl)benzonitrile (Intermediate 6b).

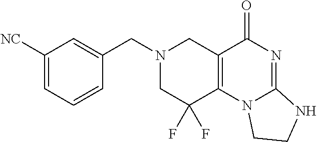 [SMILES: N#Cc1cccc(CN2Cc3c(n4c(nc3=O)NCC4)C(F)(F)C2)c1]

To a solution of Intermediate 5b: 3-((9, 9-difluoro-4-(4-methoxybenzyl)-5-oxo-1, 2, 4, 5, 8, 9-hexahydroimidazo[1,2-a]pyrido[3,4-e]pyri midin-7(6H)-yl)methyl)benzonitrile (2.5 g, 7.3 mmol) in dry dichloromethane (100 mL) was added dry aluminum trichloride (2.9 g, 21.8 mmol) under nitrogen protectionand stirred at room temperature overnight. TLC showed that the reaction was completed. the reaction solution was quenched with water and adjusted to stong alkaline with sodium hydroxide solution The mixture was partitioned. The aqueous layer was extracted three times with methanol:chloroform (1:9), and the combined organic phases were washed three times with water, once with saturated salt water, and dried over anhydrous sodium sulfate. After filtration, the filtrate was concentrated under reduced pressure and the residue was suspended in ethyl acetate to beat. A white solid (1.1 g, 3.2 mmol) was obtained after filtration with a yield of 44%. ¹H NMR (400 MHZ, DMSO-d₆) δ 7.84-7.55 (m, 4H), 4.17 (dd, J=9.7, 7.5 Hz, 2H), 3.82 (s, 2H), 3.62 (dd, J=9.7, 7.5 Hz, 2H), 3.21-3.12 (m, 4H). HRMS (ESI): calcd. for [M+H]⁺344.1323, found 344.1332.

### Example 77: Preparation of 7-Benzyl-9, 9-difluoro-3-(2-methylbenzyl)-2,3,6,7,8,9-hexahydroimidazo[1,2-a]pyrido[3,4-e]pyrimid in-5(1H)-one (Compound 47).

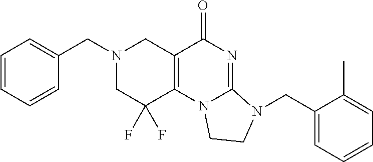 [SMILES: Cc1ccccc1CN1CCn2c1nc(=O)c1c2C(F)(F)CN(Cc2ccccc2)C1]

To a solution of Intermediate 6a: 7-Benzyl-9, 9-difluoro-2, 4, 6, 7, 8, 9-hexahydroimidazo[1,2-a]pyrido[3,4-e]pyrimidin-5(1H)-one (50 mg, 0.16 mmol) in 10 mL of dry acetonitrile was added successively anhydrous potassium carbonate (65 mg, 0.47 mmol) and 2-methylbenzyl bromide (59 mg, 0.32 mmol) under nitrogen protection. The reaction mixture was heated to 50° C. and stirred overnight. The completion of the reaction was monitored by TLC, and then excess methanol was added to quench the reaction for 1 hour. After filtration, the filtrate was concentrated under reduced pressure. The residue was subjected to silica gel column chromatography, eluting with methanol:dichloromethane (7:93). The resulting product was obtained as a white solid (35 mg, 0.08 mmol) after concentration under reduced pressure. The yield was 52%.¹H NMR (400 MHZ, DMSO-d₆) δ 7.43-7.14 (m, 9H), 4.51 (s, 2H), 4.16 (dd, J=9.6, 7.6 Hz, 2H), 3.75 (s, 2H), 3.53 (dd, J=9.8, 7.4 Hz, 2H), 3.24-3.08 (m, 4H), 2.28 (s, 3H). ¹³C NMR (101 MHz, DMSO-d₆) δ 169.02, 157.41, 137.51, 136.75, 134.82(t, J=24.2 Hz), 134.40, 130.78, 129.29, 128.88, 128.50, 128.05, 127.87, 126.44, 116.21(t, J=241.5 Hz), 116.10 (t, J=6.6 Hz), 60.36, 57.24 (t, J=26.8 Hz), 49.35, 45.95, 45.16, 44.58, 19.15.HRMS (ESI⁺): m/z calcd for C24H27F2N₄O [M+H]⁺423.1996, found 423.1994.

### Example 78: Preparation of 7-Benzyl-9, 9-difluoro-3-(3-methylbenzyl)-2,3,6,7,8,9-hexahydroimidazo[1,2-a]pyrido[3,4-e]pyrimid in-5(1H)-one (Compound 48)

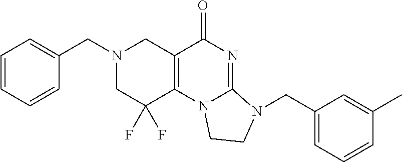 [SMILES: Cc1cccc(CN2CCn3c2nc(=O)c2c3C(F)(F)CN(Cc3ccccc3)C2)c1]

Apart from the substitution of the key intermediate, the synthesis steps of the target product were the same as in Example 77 wherein the ingredient was 3-methylbenzyl bromide, resulting in a white solid with a yield of 44%. ¹H NMR (400 MHZ, DMSO-d₆) δ 7.45-7.19 (m, 6H), 7.18-7.06 (m, 3H), 4.49 (s, 2H), 4.15 (dd, J=9.7, 7.5 Hz, 2H), 3.75 (s, 2H), 3.56 (dd, J=9.8, 7.5 Hz, 2H), 3.25-3.18 (m, 2H), 3.17-3.08 (m, 2H), 2.30 (s, 3H). HRMS (ESI): calcd. for [M+H]⁺423.1996, found 423.1993.

### Example 79: Preparation of 7-Benzyl-9, 9-difluoro-3-(3-bromo-4-fluorobenzyl)-2,3,6,7,8,9-hexahydroimidazo[1,2-a]pyrido[3,4-e ]pyrimidin-5(1H)-one (Compound 49)

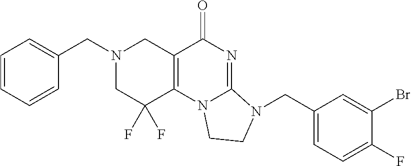 [SMILES: O=c1nc2n(c3c1CN(Cc1ccccc1)CC3(F)F)CCN2Cc1ccc(F)c(Br)c1]

Apart from the substitution of the key intermediate, the synthesis steps of the target product were the same as in Example 77 wherein the ingredient was 3-bromo-4-fluorobenzyl bromide, resulting in a white foam solid with a yield of 60%. ¹H NMR (400 MHZ, DMSO-d₆) δ 7.70 (dd, J=6.8, 2.0 Hz, 1H), 7.44-7.26 (m, 7H), 4.51 (d, J=2.5 Hz, 2H), 4.16 (dd, J=9.7, 7.5 Hz, 2H), 3.76 (s, 2H), 3.60 (dd, J=9.8, 7.4 Hz, 2H), 3.24-3.08 (m, 4H). HRMS (ESI): calcd. for [M+Na]⁺527.0670, found527.0677.

### Example 80: Preparation of 3-((3-(3,4-difluorobenzyl)-9, 9-difluoro-5-oxo-2,3,6,7,8,9-hexahydroimidazo[1,2-a]pyrido[3,4-e]pyri midin-7(6H)-yl)methyl)benzonitrile (Compound 50)

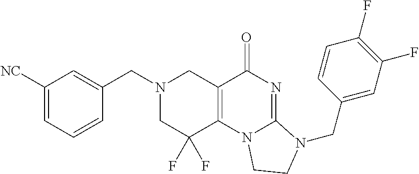 [SMILES: N#Cc1cccc(CN2Cc3c(n4c(nc3=O)N(Cc3ccc(F)c(F)c3)CC4)C(F)(F)C2)c1]

Apart from the substitution of the key intermediate, the synthesis steps of the target product were the same as in Example 77 wherein the ingredient was intermediate 6b and 3,4-difluorobenzyl bromide, resulting in a white foam solid with a yield of 65%. ¹H NMR (400 MHZ, DMSO-d₆) δ 7.78 (dq, J=4.7, 1.5 Hz, 2H), 7.73-7.65 (m, 1H), 7.59 (t, J=7.9 Hz, 1H), 7.49-7.34 (m, 2H), 7.20 (ddd, J=10.1, 4.8, 2.1 Hz, 1H), 4.52 (s, 2H), 4.25-4.11 (m, 2H), 3.83 (s, 2H), 3.66-3.56 (m, 2H), 3.23 (t, J=4.4 Hz, 2H), 3.17 (t, J=12.2 Hz, 2H). HRMS (ESI): calcd. for [M+H]⁺470.1604, found 470.1601.

### Example 81: Preparation of methyl 3-((7-benzyl-9, 9-difluoro-5-oxo-1, 2, 6, 7, 8, 9-hexahydroimidazo[1,2-a]pyrido[3,4-e]pyrimidin-3(5H)-yl)methyl)benzoate (Compound 51)

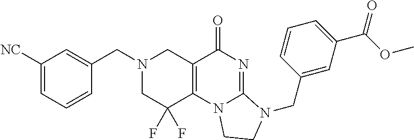 [SMILES: COC(=O)c1cccc(CN2CCn3c2nc(=O)c2c3C(F)(F)CN(Cc3cccc(C#N)c3)C2)c1]

Apart from the substitution of the key intermediate, the synthesis steps of the target product were the same as in Example 77 wherein the ingredient was intermediate 6b and methyl 3-bromomethylbenzoate, resulting in a white foam solid with a yield of 60%. ¹H NMR (400 MHZ, DMSO-d₆) δ 7.98-7.86 (m, 2H), 7.84-7.74 (m, 2H), 7.69 (dt, J=7.8, 1.5 Hz, 1H), 7.66-7.50 (m, 3H), 4.61 (s, 2H), 4.17 (dd, J=9.7, 7.5 Hz, 2H), 3.86 (s, 3H), 3.84 (s, 2H), 3.60 (dd, J=9.7, 7.4 Hz, 2H), 3.25 (t, J=4.5 Hz, 2H), 3.22-3.11 (m, 2H). HRMS (ESI): calcd. for [M+H]⁺492.1847, found 492.1844.

### Example 82: Preparation of 3-((3-benzyl-9, 9-difluoro-5-oxo-2,3,6,7,8,9-hexahydroimidazo[1,2-a]pyrido[3,4-e]pyrimidin-7(6H)-yl)methyl)benzonitrile (Compound 52).

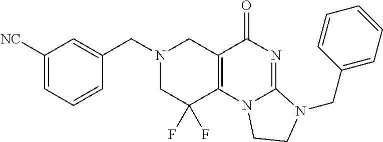 [SMILES: N#Cc1cccc(CN2Cc3c(n4c(nc3=O)N(Cc3ccccc3)CC4)C(F)(F)C2)c1]

Apart from the substitution of the key intermediate, the synthesis steps of the target product were the same as in Example 77 wherein the ingredient was intermediate 6b and benzyl bromide, resulting in a yield of 48%. ¹H NMR (400 MHZ, DMSO-d₆) δ 7.86-7.75 (m, 2H), 7.69 (dd, J=7.9, 1.6 Hz, 1H), 7.59 (t, J=7.9 Hz, 1H), 7.45-7.27 (m, 5H), 4.54 (s, 2H), 4.17 (dd, J=9.7, 7.5 Hz, 2H), 3.83 (s, 2H), 3.58 (dd, J=9.7, 7.5 Hz, 2H), 3.24 (t, J=4.5 Hz, 2H), 3.21-3.11 (m, 2H). HRMS (ESI): calcd. for [M+H]⁺434.1792, found 434.1786.

### Example 83: Preparation of 3-((3-(3-fluorobenzyl)-9, 9-difluoro-5-oxo-2,3,6,7,8,9-hexahydroimidazo[1,2-a]pyrido[3,4-e]pyrimid in-7(6H)-yl)methyl)benzonitrile (Compound 53)

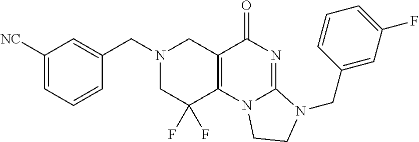 [SMILES: N#Cc1cccc(CN2Cc3c(n4c(nc3=O)N(Cc3cccc(F)c3)CC4)C(F)(F)C2)c1]

Apart from the substitution of the key intermediate, the synthesis steps of the target product were the same as in Example 77 with the ingredient being intermediate 6b and 3-fluorobenzyl bromide, resulting in a white foam solid with a yield of50%. ¹H NMR (400 MHZ, DMSO-d₆) δ 7.90 -7.75 (m, 2H), 7.72-7.65 (m, 1H), 7.63-7.55 (m, 1H), 7.47-7.36 (m, 1H), 7.27-7.07 (m, 3H), 4.56 (s, 2H), 4.19 (dd, J=9.7, 7.5 Hz, 2H), 3.84 (s, 2H), 3.62 (dd, J=9.8, 7.4 Hz, 2H), 3.24 (t, J=4.4 Hz, 2H), 3.21-3.12 (m, 2H). HRMS (ESI): calcd. for [M+H]⁺452.1698, found 452.1689.

### Example 84: Preparation of 3-((3-(3-bromo-4-fluorobenzyl)-9, 9-difluoro-5-oxo-2,3,6,7,8,9-hexahydroimidazo[1,2-a]pyrido[3,4-e]pyrimidin-7(6H)-yl)methyl)benzonitrile (Compound 54)

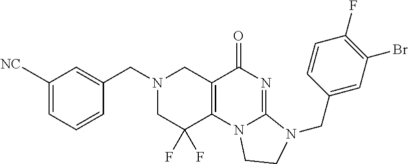 [SMILES: N#Cc1cccc(CN2Cc3c(n4c(nc3=O)N(Cc3ccc(F)c(Br)c3)CC4)C(F)(F)C2)c1]

Apart from the substitution of the key intermediate, the synthesis steps of the target product were the same as in Example 77 with the ingredient being intermediate 6b and 3-bromo-4-fluorobenzyl bromide, resulting in a white foam solid with a yield of58%. ¹H NMR (400 MHz, DMSO-d₆) δ 7.83-7.76 (m, 2H), 7.70 (td, J=6.4, 1.7 Hz, 2H), 7.59 (t, J=7.9 Hz, 1H), 7.44-7.33 (m, 2H), 4.52 (s, 2H), 4.17 (dd, J=9.7, 7.5 Hz, 2H), 3.83 (s, 2H), 3.60 (t, J=8.6 Hz, 2H), 3.23 (t, J=4.5 Hz, 2H), 3.21-3.14 (m, 3H). HRMS (ESI): calcd. for [M+H]⁺530.0803, found530.0801.

### Example 85: Preparation of 3-((9, 9-difluoro-3-isopentyl-5-oxo-2,3,6,7,8,9-hexahydroimidazo[1,2-a]pyrido[3,4-e]pyrimidin-7(6H)-yl)methyl)benzonitrile (Compound 55)

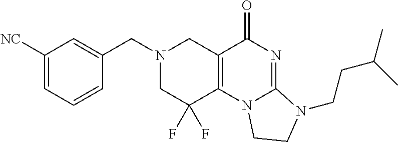 [SMILES: CC(C)CCN1CCn2c1nc(=O)c1c2C(F)(F)CN(Cc2cccc(C#N)c2)C1]

Apart from the substitution of the key intermediate, the synthesis steps of the target product were the same as in Example 77 with the ingredient being intermediate 6b and 1-bromoisopentane, resulting in a yield of35%. ¹H NMR (400 MHZ, DMSO-d₆) δ 7.81-7.74 (m, 2H), 7.71-7.65 (m, 1H), 7.62-7.55 (m, 1H), 4.14 (dd, J=9.8, 7.5 Hz, 2H), 3.82 (s, 2H), 3.67 (dd, J=9.9, 7.5 Hz, 2H), 3.39-3.27 (m, 2H), 3.25-3.19 (m, 2H), 3.19-3.10 (m, 2H), 1.58 (dp, J=13.3, 6.6 Hz, 1H), 1.49-1.36 (m, 2H), 0.91 (s, 3H), 0.90 (s, 3H). HRMS (ESI): calcd. for [M+H]⁺414.2105, found 414.2104.

### Example 86: Preparation of 3-((9, 9-difluoro-3-(4-methylbenzyl)-5-oxo-2,3,6,7,8,9-hexahydroimidazo[1,2-a]pyrido[3,4-e]pyrimi din-7(6H)-yl)methyl)benzonitrile (Compound 56)

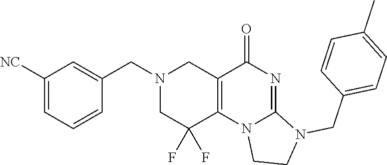 [SMILES: Cc1ccc(CN2CCn3c2nc(=O)c2c3C(F)(F)CN(Cc3cccc(C#N)c3)C2)cc1]

Apart from the substitution of the key intermediate, the synthesis steps of the target product were the same as in Example 77 with the ingredient being intermediate 6b and 4-methylbenzyl bromide, resulting in a white solid with a yield of 41%. ¹H NMR (400 MHZ, DMSO-d₆) δ 7.78 (dt, J =5.5, 1.7 Hz, 2H), 7.69 (dt, J=7.9, 1.5 Hz, 1H), 7.59 (t, J=7.9 Hz, 1H), 7.28-7.11 (m, 4H), 4.48 (s, 2H), 4.15 (dd, J=9.7, 7.5 Hz, 2H), 3.83 (s, 2H), 3.55 (dd, J=9.7, 7.5 Hz, 2H), 3.24 (t, J=4.5 Hz, 2H), 3.20-3.07 (m, 2H), 2.29 (s, 3H). HRMS (ESI): calcd. for [M+H]⁺448.1949, found 448.1947.

### Example 87: Preparation of 3-((9, 9-difluoro-3-(3,4-dichlorobenzyl)-5-oxo-2,3,6,7,8,9-hexahydroimidazo[1,2-a]pyrido[3,4-e]pyri midin-7(6H)-yl)methyl)benzonitrile (Compound 57)

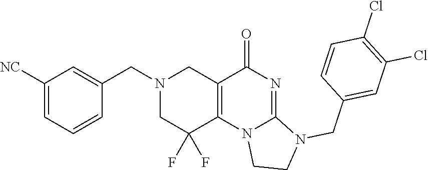 [SMILES: N#Cc1cccc(CN2Cc3c(n4c(nc3=O)N(Cc3ccc(Cl)c(Cl)c3)CC4)C(F)(F)C2)c1]

Apart from the substitution of the key intermediate, the synthesis steps of the target product were the same as in Example 77 with the ingredient being intermediate 6b and 3,4-dichlorobenzyl bromide, resulting in a white solid with a yield of 60%. ¹H NMR (400 MHZ, DMSO-d₆) δ 7.82-7.74 (m, 2H), 7.73-7.54 (m, 4H), 7.34 (dd, J=8.3, 2.0 Hz, 1H), 4.54 (s, 2H), 4.18 (dd, J=9.7, 7.5 Hz, 2H), 3.84 (s, 2H), 3.62 (dd, J=9.8, 7.4 Hz, 2H), 3.28-3.12 (m, 4H).HRMS (ESI): calcd. for [M+H]⁺502.1013, found502.1007.

### Example 88: Preparation of 3-((9, 9-difluoro-3-(3-methylbenzyl)-5-oxo-2,3,6,7,8,9-hexahydroimidazo[1,2-a]pyrido[3,4-e]pyrimi din-7(6H)-yl)methyl)benzonitrile (Compound 58)

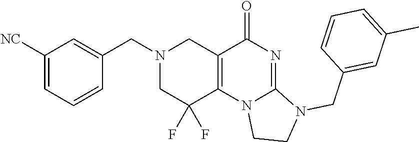 [SMILES: Cc1cccc(CN2CCn3c2nc(=O)c2c3C(F)(F)CN(Cc3cccc(C#N)c3)C2)c1]

Apart from the substitution of the key intermediate, the synthesis steps of the target product were the same as in Example 77 with the ingredient being intermediate 6b and 3-methylbenzyl bromide, resulting in a white solid with a yield of 44%. ¹H NMR (400 MHZ, DMSO-d₆) δ 7.42-7.20 (m, 6H), 7.16-7.08 (m, 3H), 4.49 (s, 2H), 4.15 (dd, J=9.7, 7.5 Hz, 2H), 3.75 (s, 2H), 3.56 (dd, J=9.8, 7.5 Hz, 2H), 3.28-3.08 (m, 4H), 2.30 (s, 3H). HRMS (ESI): calcd. for [M+H]⁺448.1949, found 448.1943 .

### Example 89: 7-Benzyl-3-(3-bromobenzyl)-2,3,6,7,8,9-hexahydroimidazo[1,2-a]pyrido[3,4-e]pyrimidin-5(1H)-one (Compound 59)

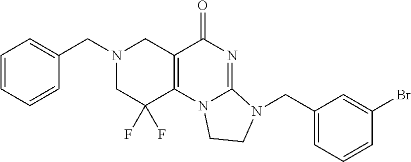 [SMILES: O=c1nc2n(c3c1CN(Cc1ccccc1)CC3(F)F)CCN2Cc1cccc(Br)c1]

Apart from the substitution of the key intermediate, the synthesis steps of the target product were the same as in Example 26, resulting in a white solid with a yield of 35%. ¹H NMR (400 MHZ, Chloroform-d) δ 7.46-7.37 (m, 2H), 7.37-7.14 (m, 7H), 4.57 (s, 2H), 3.90 (dd, J=9.7, 7.3 Hz, 2H), 3.69 (s, 2H), 3.49 (dd, J=9.8, 7.3 Hz, 2H), 3.40 (s, 2H), 2.76-2.63 (m, 2H), 2.56-2.43 (m, 2H). ¹³C NMR (101 MHz, Chloroform-d) δ 171.16, 171.01, 156.47, 142.48, 138.21, 137.79, 131.27, 131.10, 130.42, 129.12 (2C), 128.38 (2C), 127.32, 127.14, 122.78, 111.12, 62.28, 50.18, 48.04, 47.53, 44.04, 42.30, 25.52. HRMS (ESI⁺): m/z calcd for C₂₃H₂₄BrN₄O [M+H]⁺451.1133, found 451.1132.

### Example 90: 7-Benzyl-3-(3-chlorobenzyl)-2,3,6,7,8,9-hexahydroimidazo[1,2-a]pyrido[3,4-e]pyrimidin-5(1H)-one (Compound 60)

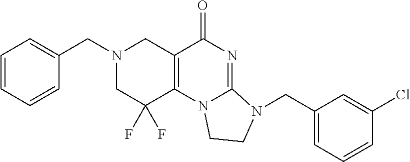 [SMILES: O=c1nc2n(c3c1CN(Cc1ccccc1)CC3(F)F)CCN2Cc1cccc(Cl)c1]

The synthesis steps of the target product were the same as in Example 26, resulting in a white solid with a yield of 37%. ¹H NMR (400 MHZ, Chloroform-d) δ 7.36-7.27 (m, 5H), 7.26-7.15 (m, 4H), 4.57 (s, 2H), 3.90 (dd, J=9.8, 7.3 Hz, 2H), 3.68 (s, 2H), 3.49 (dd, J=9.7, 7.4 Hz, 2H), 3.43 -3.35 (m, 2H), 2.74-2.63 (m, 2H), 2.54-2.42 (m, 2H).¹³C NMR (101 MHz, Chloroform-d) 8 171.02, 156.48, 142.52, 137.95, 137.84, 134.57, 130.12, 129.09 (2C), 128.36 (2C), 128.13, 127.29, 126.64, 111.07, 62.29, 50.19, 48.06, 47.55, 44.03, 42.30, 25.52. HRMS (ESI⁺): m/z calcd for C₂₃H₂₄ClN₄O [M+H]⁺407.1639, found 407.1640.

### Example 91: 7-Benzyl-3-(2-chlorobenzyl)-2,3,6,7,8,9-hexahydroimidazo[1,2-a]pyrido[3,4-e]pyrimidin-5(1H)-one (Compound 61)

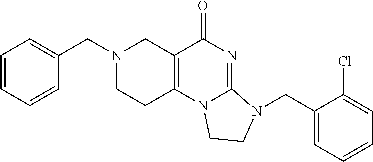 [SMILES: O=c1nc2n(c3c1CN(Cc1ccccc1)CC3)CCN2Cc1ccccc1Cl]

Apart from the substitution of the key intermediate, the synthesis steps of the target product were the same as in Example 26, resulting in a white solid with a yield of 36%. ¹H NMR (400 MHZ, Chloroform-d) δ 7.46-7.40 (m, 1H), 7.39-7.27 (m, 5H), 7.26-7.20 (m, 3H), 4.77 (s, 2H), 3.91 (dd, J=9.8, 7.3 Hz, 2H), 3.68 (s, 2H), 3.56 (dd, J=9.8, 7.3 Hz, 2H), 3.40 (s, 2H), 2.76-2.62 (m, 2H), 2.55-2.41 (m, 2H). ¹³C NMR (101 MHZ, Chloroform-d) δ 171.06, 156.55, 142.45, 137.89, 133.90, 133.44, 130.53, 129.59, 129.32, 129.08 (2C), 128.36 (2C), 127.37, 127.27, 111.09, 62.28, 50.23, 48.03, 45.13, 44.35, 42.37, 25.54. HRMS (ESI⁺): m/z calcd for C₂₃H₂₄Cl₄O [M+H]+407.1639, found 407.1643.

### Example 92: 7-Benzyl-3-(2-fluorobenzyl)-2,3,6,7,8,9-hexahydroimidazo[1,2-a]pyrido[3,4-e]pyrimidin-5(1H)-one (Compound 62)

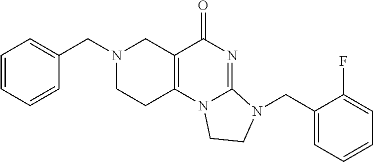 [SMILES: O=c1nc2n(c3c1CN(Cc1ccccc1)CC3)CCN2Cc1ccccc1F]

Apart from the substitution of the key intermediate, the synthesis steps of the target product were the same as in Example 26, resulting in a white solid with a yield of 33%. ¹H NMR (400 MHZ, Chloroform-d) δ 7.36-7.21 (m, 6H), 7.10-7.05 (m, 1H), 7.02-6.93 (m, 2H), 4.59 (s, 2H), 3.90 (dd, J=9.8, 7.3 Hz, 2H), 3.68 (s, 2H), 3.49 (dd, J=9.8, 7.3 Hz, 2H), 3.39 (s, 2H), 2.72-2.63 (m, 2H), 2.53-2.44 (m, 2H). ¹³C NMR (101 MHZ, Chloroform-d) δ 171.04, 162.95 (d, J=247.2 Hz), 156.52, 142.54, 138.40 (d, J=7.1 Hz), 137.85, 130.36 (d, J=8.3 Hz), 129.09 (2C), 128.36 (2C), 127.28, 124.04 (d, J=2.9 Hz), 115.19 (d, J=21.4 Hz), 114.87 (d, J=21.2 Hz), 111.06, 62.28, 50.19, 48.05, 47.59, 44.03, 42.30, 25.51. HRMS (ESI⁺): m/z calcd for C23H24FN₄O [M+H]⁺391.1934, found 391.1932.

### Example 93: 7-Benzyl-3-(3-fluorobenzyl)-2,3,6,7,8,9-hexahydroimidazo[1,2-a]pyrido[3,4-e]pyrimidin-5(1H)-one (Compound 63)

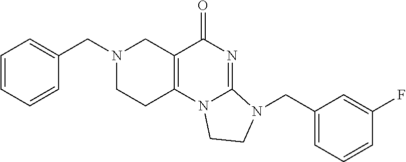 [SMILES: O=c1nc2n(c3c1CN(Cc1ccccc1)CC3)CCN2Cc1cccc(F)c1]

Apart from the substitution of the key intermediate, the synthesis steps of the target product were the same as in Example 26, resulting in a yield of 38%¹H NMR (400 MHZ, Chloroform-d) 8 7.48-7.41 (m, 1H), 7.35-7.22 (m, 6H), 7.13-7.01 (m, 2H), 4.68 (s, 2H), 3.89 (dd, J=9.8, 7.3 Hz, 2H), 3.68 (s, 2H), 3.55 (dd, J=9.8, 7.3 Hz, 2H), 3.40 (s, 2H), 2.76-2.60 (m, 2H), 2.53-2.43 (m, 2H). ¹³C NMR (101 MHz, Chloroform-d) δ 171.06, 161.13 (d, J=246.4 Hz), 156.54, 142.42, 137.87, 131.30 (d, J=3.8 Hz), 129.88 (d, J=8.1 Hz), 129.08 (2C), 128.36 (2C), 127.27, 124.68 (d, J=3.6 Hz), 122.66 (d, J=14.8 Hz), 115.34 (d, J=21.5 Hz), 111.09, 62.25, 50.21, 48.00, 44.19, 42.31, 41.16, 41.12, 25.51. HRMS (ESI⁺): m/z calcd for C₂₃H₂₄FN₄O [M+H]⁺391.1934, found 391.1934.

### Example 94: 7-Benzyl-3-(3-methoxybenzyl)-2,3,6,7,8,9-hexahydroimidazo[1,2-a]pyrido[3,4-e]pyrimidin-5(1H)-o ne (Compound 64)

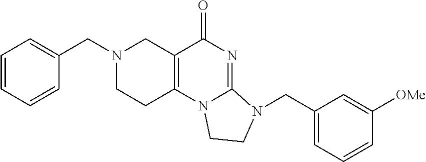 [SMILES: COc1cccc(CN2CCn3c2nc(=O)c2c3CCN(Cc3ccccc3)C2)c1]

Apart from the substitution of the key intermediate, the synthesis steps of the target product were the same as in Example 26, resulting in a white solid with a yield of 40%. ¹H NMR (400 MHZ, Chloroform-d) δ 7.36-7.18 (m, 5H), 6.89-6.78 (m, 3H), 4.56 (s, 2H), 3.86 (dd, J=9.8, 7.3 Hz, 2H), 3.77 (s, 3H), 3.68 (s, 2H), 3.47 (dd, J=9.8, 7.3 Hz, 2H), 3.42-3.37 (m, 2H), 2.71-2.64 (m, 2H), 2.51-2.43 (m, 2H). ¹³C NMR (101 MHz, Chloroform-d) δ 171.11, 159.94, 156.53, 142.46, 137.87, 137.31, 129.77 (2C), 129.10 (2C), 128.36, 127.27, 120.80, 114.03, 113.40, 110.91, 62.30, 55.31, 50.23, 48.07 (2C), 43.95, 42.26, 25.50. HRMS (ESI⁺): m/z calcd for C₂₄H₂₇N₄O₂[M+H]+403.2134, found 403.2136.

### Example 95: 7-Benzyl-3-ethyl-9, 9-difluoro-2,3,6,7,8,9-hexahydroimidazo[1,2-a]pyrido[3,4-e]pyrimidin-5(1H)-on e (Compound 65)

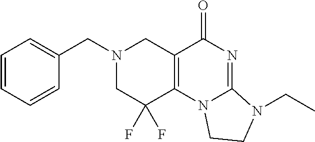 [SMILES: CCN1CCn2c1nc(=O)c1c2C(F)(F)CN(Cc2ccccc2)C1]

The synthesis of Compound 65 started from the ingredient ethyl 1-benzyl-5, 5-difluoro-4-oxo-piperidin-3-carboxylate and 1-ethylimidazolidin-2-imine hydrobromide (Intermediate 1c). The two components were dissolved in anhydrous methanol in 1:1 molar ratio, followed by the addition of sodium methoxide (4 eq) under stirring. After refluxing for 3 hours, TLC showed that the reaction was completed. The reaction solution was concentrated under reduced pressure to remove the solvent, and the resulting residue was suspended in water and extracted with dichloromethane three times The combined organic phases were washed successively with water, saturated salt water, and dried over anhydrous sodium sulfate. After filtration to remove the drying agent, the filtrate was concentrated under reduced pressure. The obtained residue was purified using a Biotage Isolera One flash column chromatography purification system equipped with silica gel columns of 200-300 mesh size to afford a white solid with a yield of 38%. ¹H NMR (400 MHZ, Chloroform-d) δ 7.40-7.22 (m, 5H), 4.28-4.11 (m, 2H), 3.75 (s, 2H), 3.72-3.63 (m, 2H), 3.58-3.43 (m, 4H), 3.10-2.94 (m, 2H), 1.17 (t, J=7.3 Hz, 3H). ¹³C NMR (101 MHZ, Chloroform-d) δ 170.02, 156.95, 136.43, 134.63 (t, J=24.2 Hz), 128.91 (2C), 128.53 (2C), 127.64, 116.50, 115.53 (t, J=241.2 Hz), 61.02, 56.98 (t, J=27.3 Hz), 49.96, 44.36, 44.17, 44.13, 44.09, 38.88, 12.03.HRMS (ESI⁺): m/z calcd for C₁₈H₂₁F₂N₄O [M+H]⁺347.1683, found 347.1682

### Example 96: 7-Benzyl-3-(cyclohexylmethyl)-9, 9-difluoro-2,3,6,7,8,9-hexahydroimidazo[1,2-a]pyrido[3,4-e]pyri midin-5(1H)-one (Compound 66)

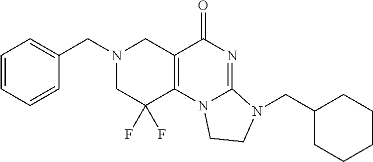 [SMILES: O=c1nc2n(c3c1CN(Cc1ccccc1)CC3(F)F)CCN2CC1CCCCC1]

Apart from the substitution of the key intermediate, the synthesis steps of the target product were the same as in Example 94, resulting in a white solid after purification with a yield of 35%. 1H NMR (400 MHZ, Chloroform-d) δ 7.35-7.30 (m, 5H), 4.20 (dd, J=9.6, 7.8 Hz, 2H), 3.76-3.73 (m, 2H), 3.67 (dd, J=9.5, 7.9 Hz, 2H), 3.49 (t, J=4.7 Hz, 2H), 3.35-3.25 (m, 2H), 3.06-2.98 (m, 2H), 1.74-1.63 (m, 6H), 1.22-1.13 (m, 3H), 1.05-0.98 (m, 2H).¹³C NMR (101 MHZ, Chloroform-d) δ 170.06, 157.61, 136.47, 134.53 (t, J=24.2 Hz), 128.93 (2C), 128.56 (2C), 127.67, 116.68, 115.60 (d, J=241.1 Hz), 61.06, 57.01(t, J=27.5 Hz), 50.37, 50.08, 45.74, 44.14, 35.90, 30.49 (2C), 26.29, 25.70 (2C).HRMS (ESI⁺): m/z calcd for C₂₃H₂₉F₂N4 [M+H]⁺415.2309, found.

### Example 97: 7-Benzyl-9, 9-difluoro-3-phenyl-2,3,6,7,8,9-hexahydroimidazo[1,2-a]pyrido[3,4-e]pyrimidin-5(1H)-one (Compound 67)

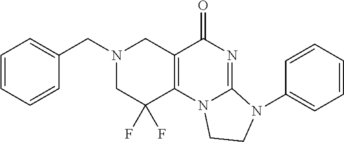 [SMILES: O=c1nc2n(c3c1CN(Cc1ccccc1)CC3(F)F)CCN2c1ccccc1]

Apart from the substitution of the key intermediate, the synthesis steps of the target product were the same as in Example 94, resulting in a white solid after purification with a yield of 39%. 1H NMR (400 MHZ, Chloroform-d) δ 7.76-7.63 (m, 2H), 7.44-7.27 (m, 7H), 7.16-7.06 (m, 1H), 4.33-4.21 (m, 2H), 4.11 (s, 2H), 3.75 (s, 2H), 3.52-3.42 (m, 2H), 3.09-2.92 (m, 2H).¹³C NMR (101 MHz, Chloroform-d) δ 169.51, 154.66, 138.29, 136.37, 134.88 (t, J=23.6 Hz), 129.05, 128.94, 128.60, 127.73, 124.44, 119.69, 119.63, 117.53, 115.47 (t, J=241.4 Hz), 61.00, 56.85(t, J=27.4 Hz), 50.00, 45.69, 43.47 (t, J=4.0 Hz). HRMS (ESI⁺): m/z calcd for C₂₂H₂₁F₂N₄O [M+H]⁺395.1683, found 395.1690

### Example 98: 3,7-Dibenzyl-9, 9-difluoro-2,3,6,7,8,9-hexahydroimidazo[1,2-a]pyrido[3,4-e]pyrimidin-5(1H)-one (Compound 68)

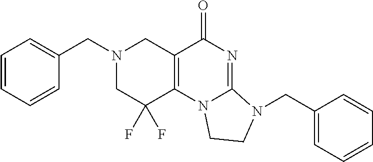 [SMILES: O=c1nc2n(c3c1CN(Cc1ccccc1)CC3(F)F)CCN2Cc1ccccc1]

Apart from the substitution of the key intermediate, the synthesis steps of the target product were the same as in Example 94, resulting in a white solid after purification with a yield of 34%. 1H NMR (400 MHZ, Chloroform-d) δ 7.46-7.17 (m, 11H), 4.64 (s, 2H), 4.20-4.10 (m, 2H), 3.75 (s, 2H), 3.59-3.47 (m, 4H), 3.06-2.93 (m, 2H).¹³C NMR (101 MHz, Chloroform-d) δ 168.98, 156.16, 135.37, 134.26, 133.65 (t, J=24.1 Hz), 127.91 (2C), 127.84 (2C), 127.55 (4C), 127.12, 126.66, 115.96, 114.50 (t, J=241.1 Hz), 76.30, 60.03, 55.95 (t, J=27.3 Hz), 49.02, 47.05, 43.28, 43.11 (t, J =4.0 Hz).HRMS (ESI⁺): m/z calcd for C₂₃H₂₃F₂N₄O [M+H]⁺409.1840, found 409.1831

### Example 99: 7-Benzyl-9, 9-difluoro-3-phenylethyl-2,3,6,7,8,9-hexahydroimidazo[1,2-a]pyrido[3,4-e]pyrimidin-5( 1H)-one (Compound 69)

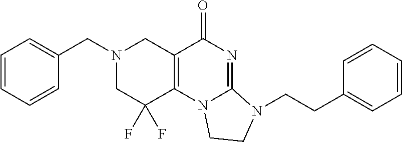 [SMILES: O=c1nc2n(c3c1CN(Cc1ccccc1)CC3(F)F)CCN2CCc1ccccc1]

Apart from the substitution of the key intermediate, the synthesis steps of the target product were the same as in Example 94, resulting in a yield of 33%. ¹H NMR (400 MHZ, Chloroform-d) 8 7.34-7.26 (m, 7H), 7.25-7.18 (m, 3H), 4.13-4.04 (m, 2H), 3.80-3.65 (m, 4H), 3.56-3.39 (m, 4H), 3.06-2.96 (m, 2H), 2.95-2.88 (m, 2H).¹³C NMR (101 MHz, Chloroform-d) δ 170.05, 157.07, 138.37, 136.41, 134.63 (t, J=24.0 Hz), 128.95 (2C), 128.76 (2C), 128.70 (2C), 128.57 (2C), 127.69, 126.70, 116.73 (t, J=240.6 Hz), 116.70 (t, J=6.1 Hz), 61.06, 56.96 (t, J=27.2 Hz), 50.04, 45.67, 45.64, 44.17 (t, J=3.8 Hz), 33.69.HRMS (ESI⁺): m/z calcd for C₂₄H₂₅F₂N₄O [M+H]⁺423.1996, found 423.1993

### Example 100: 7-Benzyl-9, 9-difluoro-3-(furan-2-ylmethyl)-2,3,6,7,8,9-hexahydroimidazo [1,2-a]pyrido[3,4-e]pyrimidin-5(1H)-one (Compound 70)

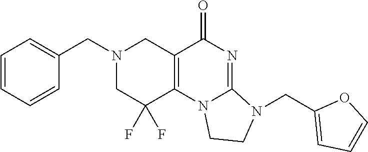 [SMILES: O=c1nc2n(c3c1CN(Cc1ccccc1)CC3(F)F)CCN2Cc1ccco1]

Apart from the substitution of the key intermediate, the synthesis steps of the target product were the same as in Example 94, resulting in a white solid after purification with a yield of 30%. 1H NMR (400 MHZ, Chloroform-d) δ 7.55-7.19 (m, 6H), 6.64-6.08 (m, 2H), 4.65 (s, 2H), 4.36 -4.06 (m, 2H), 3.93-3.60 (m, 4H), 3.60-3.39 (m, 2H), 3.21-2.88 (m, 2H).¹³C NMR (101 MHz, Chloroform-d) δ 169.89, 156.93, 149.15, 142.86, 136.40, 134.67, 128.92 (2C), 128.57 (2C), 127.70, 117.16 (t, J=240.6 Hz), 117.07, 110.59, 109.46, 61.04, 56.96, 49.99, 44.87, 44.22, 40.64. HRMS (ESI⁺): m/z calcd for C₂₁H₂₁F₂N₄O₂[M+H]⁺399.1633, found 399.1633

### Example 101: 7-Benzyl-9, 9-difluoro-3-(3-methylbenzyl)-2,3,6,7,8,9-hexahydroimidazo [1,2-a]pyrido[3,4-e]pyrimidin-5(1H)-one (Compound 71)

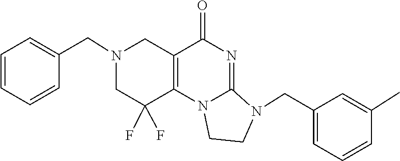 [SMILES: Cc1cccc(CN2CCn3c2nc(=O)c2c3C(F)(F)CN(Cc3ccccc3)C2)c1]

Apart from the substitution of the key intermediate, the synthesis steps of the target product were the same as in Example 94, resulting in a white solid after purification with a yield of 37%. 1H NMR (400 MHZ, DMSO-d₆) δ 7.41-7.20 (m, 6H), 7.16-7.08 (m, 3H), 4.49 (s, 2H), 4.20-4.09 (m, 2H), 3.75 (s, 2H), 3.60-3.52 (m, 2H), 3.24-3.18 (m, 2H), 3.18-3.09 (m, 2H), 2.30 (s, 3H). ¹³C NMR (101 MHz, DMSO-d₆) δ 169.02, 157.51, 138.30, 137.51, 136.55, 134.80(t, J=24.2 Hz), 129.30 (2C), 129.00, 128.89 (2C), 128.68, 127.88, 125.41, 116.19 (t, J=239.9 Hz), 116.15 (t, J=5.5 Hz), 60.35, 57.22 (t, J=26.9 Hz), 49.35, 47.47, 44.94, 44.57, 21.46.HRMS (ESI⁺): m/z calcd for C₂₄H₂₅F₂N₄O [M+H]⁺423.1996, found 423.1996

### Example 102: 7-Benzyl-9, 9-difluoro-3-(4-methylbenzyl)-2,3,6,7,8,9-hexahydroimidazo[1,2-a]pyrido[3,4-e]pyrimid in-5(1H)-one (Compound 72)

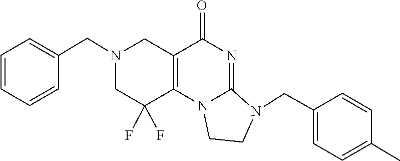 [SMILES: Cc1ccc(CN2CCn3c2nc(=O)c2c3C(F)(F)CN(Cc3ccccc3)C2)cc1]

Apart from the substitution of the key intermediate, the synthesis steps of the target product were the same as in Example 94, resulting in a white solid after purification with a yield of 33%. 1H NMR (400 MHZ, Chloroform-d) δ 7.27-7.16 (m, 6H), 7.16-7.03 (m, 4H), 4.52 (s, 2H), 4.07 (td, J =8.5, 1.9 Hz, 2H), 3.68 (s, 2H), 3.52-3.34 (m, 4H), 2.94 (t, J=12.0 Hz, 2H), 2.26 (s, 3H). ¹³C NMR (101 MHz, Chloroform-d) δ 170.03, 157.17, 137.94, 134.65 (t, J=23.6 Hz), 132.21, 129.53 (2C), 128.94 (2C), 128.63 (2C), 128.58 (2C), 127.70, 117.94, 116.96 (t, J=5.9 Hz), 115.55 (t, J=240.7 Hz), 61.07, , 128.63, 128.58, 127.70, 117.94, 116.96 (t, J=5.9 Hz), 115.55 (t, J=240.7 Hz), 61.07, , 128.63, 128.58, 127.70, 117.94, 116.96 (t, J=5.9 Hz), 115.55 (t, J=240.7 Hz), 61.07, , 128.63, 128.58, 127.70, 117.94, 116.96 (t, J=5.9 Hz), 115.55 (t, J=240.7 Hz), 61.07, 56.99 (t, J=27.3 Hz), 50.07, 47.80, 44.22, 44.13 (t, J=4.2 Hz), 21.14.HRMS (ESI⁺): m/z calcd for C₂₄H₂₅F₂N₄O [M+H]⁺423.1996, found 423.1993

### Example 103: 7-Benzyl-9, 9-difluoro-3-(4-(trifluoromethyl)benzyl)-2,3,6,7,8,9-hexahydroimidazo[1,2-a]pyrido[3,4-e]pyrimidin-5(1H)-one (Compound 73)

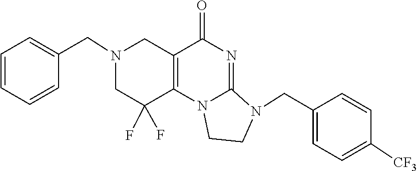 [SMILES: O=c1nc2n(c3c1CN(Cc1ccccc1)CC3(F)F)CCN2Cc1ccc(C(F)(F)F)cc1]

Apart from the substitution of the key intermediate, the synthesis steps of the target product were the same as in Example 94, resulting in a white solid after purification with a yield of 36%. 1H NMR (400 MHz, Chloroform-d) δ 7.69-7.38 (m, 5H), 7.35-7.22 (m, 4H), 4.69 (s, 2H), 4.40 -4.07 (m, 2H), 3.75 (s, 2H), 3.68-3.41 (m, 4H), 3.20-2.87 (m, 2H). ¹³C NMR (101 MHZ, Chloroform-d) δ 168.86, 156.19, 138.46, 135.32, 133.71, 129.40 (d, J=32.1 Hz), 127.91 (2C), 127.74 (2C), 127.56 (2C), 126.70, 124.85, 124.81, 122.95 (q, J=272.4 Hz), 116.25, 114.47 (t, J=240.7 Hz), 60.04, 55.93 (t, J=27.2 Hz), 48.96, 46.64, 43.49, 43.18.HRMS (ESI⁺): m/z calcd for C₂₄H₂₂F₅N₄O [M+H]⁺477.1714, found 477.1711

### Example 104: 7-Benzyl-9, 9-difluoro-3-(2-methoxybenzyl)-2,3,6,7,8,9-hexahydroimidazo[1,2-a]pyrido[3,4-e]pyrimidin-5(1H)-one (Compound 74)

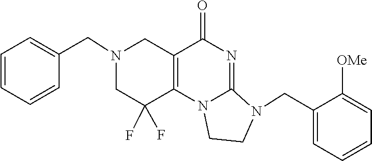 [SMILES: COc1ccccc1CN1CCn2c1nc(=O)c1c2C(F)(F)CN(Cc2ccccc2)C1]

Apart from the substitution of the key intermediate, the synthesis steps of the target product were the same as in Example 94, resulting in a white solid after purification with a yield of 29%. 1H NMR (400 MHZ, Chloroform-d) δ 7.44-7.26 (m, 7H), 6.96-6.83 (m, 2H), 4.69 (s, 2H), 4.21 -4.04 (m, 2H), 3.82 (s, 3H), 3.74 (s, 2H), 3.62-3.47 (m, 4H), 3.08-2.92 (m, 2H). ¹³C NMR (101 MHz, Chloroform-d) δ 170.10, 157.71, 157.27, 136.45, 134.65 (t, J=24.0 Hz), 130.69, 129.50, 128.94 (2C), 128.56 (2C), 127.66, 123.52, 120.90, 116.69 (t, J=6.1 Hz), 115.56 (t, J=240.7 Hz), 110.48, 61.06, 56.99 (t, J=27.4 Hz), 55.43, 50.08, 44.74, 44.18, 42.36. HRMS (ESI⁺): m/z calcd for C₂₄H₂₅F₂N₄O₂[M+H]⁺439.1946, found 439.1950

### Example 105: 7-Benzyl-9, 9-difluoro-3-(4-(methylsulfonyl)benzyl)-2,3,6,7,8,9-hexahydroimidazo[1,2-a]pyrido[3,4-e]pyrimidin-5(1H)-one (Compound 75)

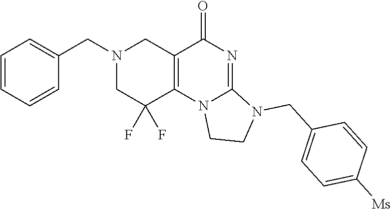 [SMILES: CS(=O)(=O)c1ccc(CN2CCn3c2nc(=O)c2c3C(F)(F)CN(Cc3ccccc3)C2)cc1]

Apart from the substitution of the key intermediate, the synthesis steps of the target product were the same as in Example 94, resulting in a white solid after purification with a yield of 31%. 1H NMR (400 MHZ, Chloroform-d) δ 7.90 (d, J=8.3 Hz, 2H), 7.52 (d, J=8.4 Hz, 2H), 7.38-7.27 (m, 5H), 4.73 (s, 2H), 4.23 (t, J=8.6 Hz, 2H), 3.76 (s, 2H), 3.60 (t, J=8.6 Hz, 2H), 3.53-3.46 (m, 2H), 3.11-2.97 (m, 5H).¹³C NMR (101 MHz, Chloroform-d) δ 169.84, 157.22, 141.92, 140.28, 136.33, 134.79 (d, J=24.2 Hz), 129.14 (2C), 128.94 (2C), 128.59 (2C), 127.97 (2C), 127.73, 117.27 (t, J=6.2 Hz), 115.47 (d, J=240.9 Hz), 61.05, 56.97 (t, J=27.3 Hz), 49.94, 47.59, 44.72, 44.47, 44.27.HRMS (ESI⁺): m/z calcd for C₂₄H₂₅F₂N₄O₃[M+H]⁺487.1615, found 487.1623

### Example 106: 7-Benzyl-9, 9-difluoro-4-(4-fluorobenzyl)-2, 4, 6, 7, 8, 9-hexahydroimidazo[1,2-a]pyrido[3,4-e]pyrimidin-5(1H)-one (Compound 76)

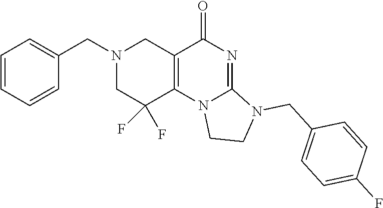 [SMILES: O=c1nc2n(c3c1CN(Cc1ccccc1)CC3(F)F)CCN2Cc1ccc(F)cc1]

Apart from the substitution of the key intermediate, the synthesis steps of the target product were the same as in Example 94, resulting in a white solid after purification with a yield of 38%. 1H NMR (400 MHz, Chloroform-d) δ 7.39-7.31 (m, 5H), 7.31-7.28 (m, 2H), 7.07-6.98 (m, 2H), 4.62 (s, 2H), 4.22-4.13 (m, 2H), 3.76 (s, 2H), 3.57-3.48 (m, 4H), 3.08-2.97 (m, 2H).¹³C NMR (101 MHz, Chloroform-d) δ 169.94, 162.60 (d, J=247.1 Hz), 157.14, 136.35, 134.68(t, J=24.2 Hz), 131.13 (d, J=3.3 Hz), 130.35 (d, J=8.1 Hz, 2C), 128.95 (2C), 128.59 (2C), 127.73, 117.14, 115.81 (d, J=21.4 Hz, 2C), 115.53, 61.07, 57.24, 56.97, 56.70, 50.03, 47.37, 44.29, 44.18, 44.14, 44.11.HRMS (ESI⁺): m/z calcd for C₂₃H₂₂F₃N₄O [M+H]⁺427.1746, found 427.1749

### Example 107:

7-Benzyl-3-(4-chlorobenzyl)-9, 9-difluoro-2,3,6,7,8,9-hexahydroimidazo[1,2-a]pyrido[3,4-e]pyrimidi n-5(1H)-one (Compound 77)

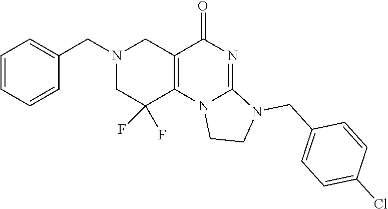 [SMILES: O=c1nc2n(c3c1CN(Cc1ccccc1)CC3(F)F)CCN2Cc1ccc(Cl)cc1]

Apart from the substitution of the key intermediate, the synthesis steps of the target product were the same as in Example 94, resulting in a white solid after purification with a yield of 37%. 1H NMR (400 MHZ, Chloroform-d) δ 7.37-7.22 (m, 9H), 4.61 (s, 2H), 4.24-4.09 (m, 2H), 3.75 (s, 2H), 3.59-3.42 (m, 4H), 3.10-2.94 (m, 2H).¹³C NMR (101 MHz, Chloroform-d) δ 169.91, 157.15, 136.37, 134.69(d, J=24.1 Hz), 134.08, 133.87, 129.93 (2C), 129.06 (2C), 128.94 (2C), 128.59 (2C), 127.72, 117.91, 115.51(t, J=241.2 Hz), 61.07, 56.97 (t, J=27.2 Hz), 50.02, 47.42, 44.34, 44.17(t, J=4.2 Hz).HRMS (ESI⁺): m/z calcd for C23H22CIF2N₄O [M+H]⁺443.1450, found 443.1447

### Example 108: 7-Benzyl-3-(4-bromobenzyl)-9, 9-difluoro-2,3,6,7,8,9-hexahydroimidazo[1,2-a]pyrido[3,4-e]pyrimidin-5(1H)-one (Compound 78)

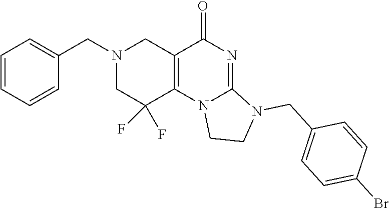 [SMILES: O=c1nc2n(c3c1CN(Cc1ccccc1)CC3(F)F)CCN2Cc1ccc(Br)cc1]

Apart from the substitution of the key intermediate, the synthesis steps of the target product were the same as in Example 94, resulting in a white solid after purification with a yield of 35%. 1H NMR (400 MHZ, Chloroform-d) δ 7.46 (d, J=8.1 Hz, 2H), 7.33 (d, J=4.8 Hz, 5H), 7.19 (d, J=8.1 Hz, 2H), 4.59 (s, 2H), 4.25-4.13 (m, 2H), 3.76 (s, 2H), 3.62-3.44 (m, 4H), 3.12-2.92 (m, 2H).¹³C NMR (101 MHz, Chloroform-d) δ 169.90, 157.14, 136.36, 134.70 (d, J=24.0 Hz), 134.39, 132.02 (2C), 130.26 (2C), 128.94 (2C), 128.59 (2C), 127.72, 122.19, 117.15 (t, J=6.0 Hz), 115.50 (d, J=241.3 Hz), 61.07, 56.96 (t, J=27.2 Hz), 50.01, 47.48, 44.35, 44.17.HRMS (ESI⁺): m/z calcd for C₂₃H₂₂BrF₂N4O [M+H]⁺487.0945, found 487.0952

### Example 109: 7-Benzyl-3-(3,4-dichlorobenzyl)-9, 9-difluoro-2,3,6,7,8,9-hexahydroimidazo[1,2-a]pyrido[3,4-e]pyrimidin-5(1H)-one (Compound 79)

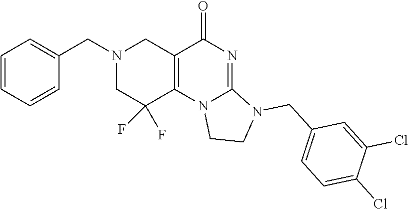 [SMILES: O=c1nc2n(c3c1CN(Cc1ccccc1)CC3(F)F)CCN2Cc1ccc(Cl)c(Cl)c1]

Apart from the substitution of the key intermediate, the synthesis steps of the target product were the same as in Example 94, resulting in a white solid after purification with a yield of 35%. 1H NMR (400 MHZ, Chloroform-d) δ 7.45-7.38 (m, 2H), 7.36-7.29 (m, 5H), 7.18 (dd, J=8.2, 2.1 Hz, 1H), 4.60 (s, 2H), 4.21 (t, J=8.6 Hz, 2H), 3.56 (t, J=8.6 Hz, 2H), 3.53-3.47 (m, 2H), 3.07 -2.97 (m, 2H).¹³C NMR (101 MHz, Chloroform-d) δ 169.84, 157.11, 136.32, 135.70, 134.71(d, J=24.3 Hz), 132.99, 132.38, 130.94, 130.28, 128.94 (2C), 128.60 (2C), 127.83, 127.74, 117.34, 115.49(d, J=241.3 Hz), 61.07, 56.94 (t, J=27.2 Hz), 49.98, 47.10, 44.48, 44.22.HRMS (ESI⁺): m/z calcd for C₂₃H₂₁C₁₂F₂N₄O [M+H]⁺477.1060, found 477.1063

### Example 110: 7-Benzyl-3-(3,4-difluorobenzyl)-9, 9-difluoro-2,3,6,7,8,9-hexahydroimidazo[1,2-a]pyrido[3,4-e]pyrimidin-5(1H)-one (Compound 80)

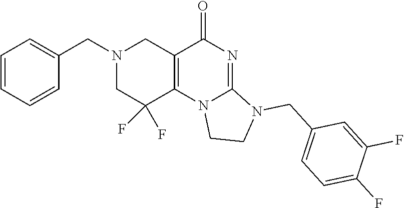 [SMILES: O=c1nc2n(c3c1CN(Cc1ccccc1)CC3(F)F)CCN2Cc1ccc(F)c(F)c1]

Apart from the substitution of the key intermediate, the synthesis steps of the target product were the same as in Example 94, resulting in a white solid after purification with a yield of 36%. 1H NMR (400 MHZ, Chloroform-d) δ 7.38-7.29 (m, 5H), 7.18-7.10 (m, 2H), 7.08-7.02 (m, 1H), 4.60 (s, 2H), 4.20 (t, J=8.6 Hz, 2H), 3.76 (s, 2H), 3.56 (t, J=8.6 Hz, 2H), 3.53-3.46 (m, 2H), 3.08-2.97 (m, 2H).¹³C NMR (101 MHz, Chloroform-d) δ 169.93, 157.11, 150.45 (dd, J=249.6, 12.9 Hz), 150.17 (dd, J=249.0, 12.7 Hz) 136.34, 134.77 (t, J=23.8 Hz), 132.43, 128.94 (2C), 128.59 (2C), 127.73, 124.59 (dd, J=6.5, 3.8 Hz), 117.57 (dd, J=28.0, 17.5 Hz, 2C), 117.25, 115.47 (t, J=240.7 Hz), 61.05, 56.93 (t, J=27.4 Hz), 49.96, 47.18, 44.42, 44.20.HRMS (ESI⁺): m/z calcd for C₂₃H₂₁F₄N₄O [M+H]⁺445.1651, found 445.1654

### Example 111: 7-Benzyl-3-(2, 4-difluorobenzyl)-9, 9-difluoro-2,3,6,7,8,9-hexahydroimidazo[1,2-a]pyrido[3,4-e]pyrimidin-5(1H)-one (Compound 81)

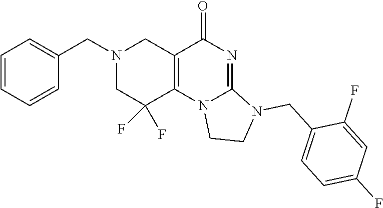 [SMILES: O=c1nc2n(c3c1CN(Cc1ccccc1)CC3(F)F)CCN2Cc1ccc(F)cc1F]

Apart from the substitution of the key intermediate, the synthesis steps of the target product were the same as in Example 94, resulting in a white solid after purification with a yield of 33%. 1H NMR (400 MHZ, Chloroform-d) δ 7.53-7.42 (m, 1H), 7.36-7.29 (m, 4H), 7.28-7.23 (m, 1H), 6.90-6.77 (m, 2H), 4.68 (s, 2H), 4.24-4.11 (m, 2H), 3.75 (s, 2H), 3.66-3.54 (m, 2H), 3.54-3.42 (m, 2H), 3.09-2.93 (m, 2H).¹³C NMR (101 MHz, Chloroform-d) δ 168.84, 161.78 (dd, J=250.4, 12.0 Hz), 160.17 (dd, J=248.8, 11.9 Hz), 156.09, 135.35, 133.68 (t, J=24.2 Hz), 131.38 (dd, J=9.8, 5.4 Hz), 127.89 (2C), 127.55 (2C), 126.67, 117.36 (dd, J 15.4, 3.8 Hz), 116.08(t, J=6.5 Hz), 114.46 (t, J=241.0 Hz), 111.00 (dd, J 21.2, 3.7 Hz), 102.84 (t, J=25.7 Hz), 60.01, 55.92 (t, J=27.3 Hz), 48.97, 43.59, 43.18 (t, J=3.8 Hz), 39.70 (d, J=3.0 Hz).HRMS (ESI): m/z calcd for C₂₃H₂₁F₄N₄O [M+H] 445.1651, found 445.1654

The structural characteristics and beneficial effects of the compounds of the present invention are elucidated through the following experimental example.

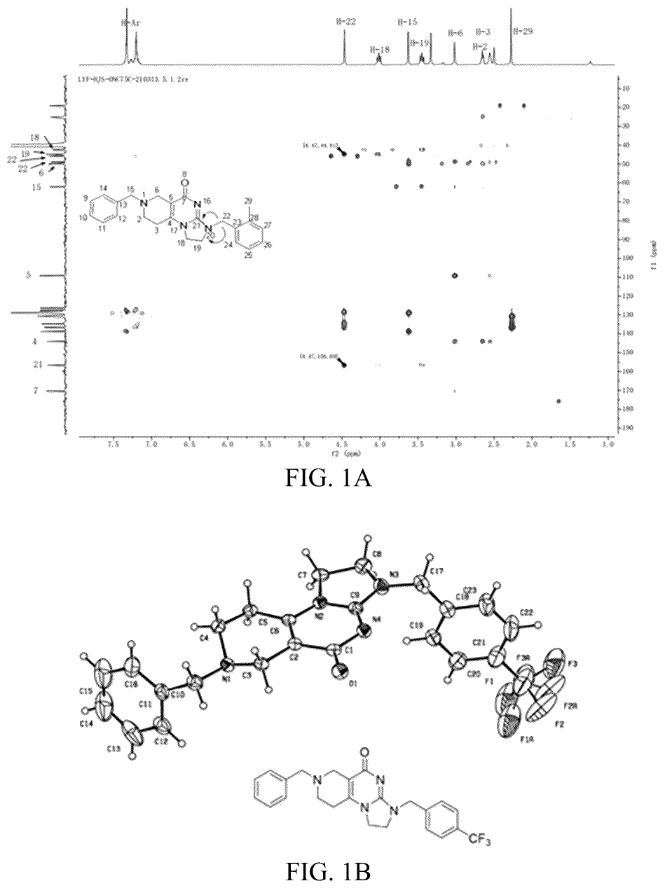

Example 1: Characterization of the structural features of the compounds of the present invention was performed through the Heteronuclear Multiple Bond Correlation spectroscopy (HMBC) of compound 1 and the single crystal diffraction pattern of compound 18. From FIG. 1A, it can be observed that the methylene hydrogen of the 2-methylbenzyl group has coupling signals with C-19 and C-21 of the imidazoline ring, while there is no correlation coupling signal with C-7 of the pyrimidinone, indicating that the substituent of this type of compound is connected to the nitrogen atom of the imidazoline ring. Traditional imipridone compounds have a skeleton of imidazolidinodihydropyrimidinone, with the substituents connected to the nitrogen atom of dihydropyrimidinone. The single crystal diffraction structure of compound 18 in FIG. 1B further verifies the structural characteristics of the compounds in the present invention. Therefore, the compounds of the present invention are a class of imidazolidinopyrimidone compounds with novel structural features.

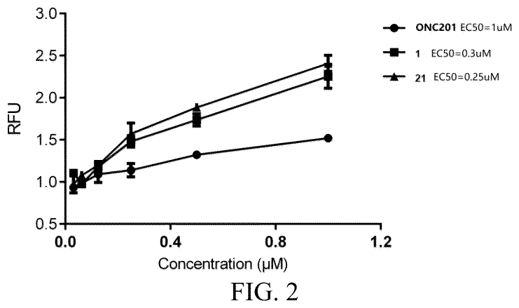

Example 2: Experiment on the activation of HsClpP enzyme hydrolysis of short peptide substrates. This study evaluated the regulatory activity of the compounds on HsClpP by examining the hydrolysis effects of HsClpP protein on the AC-WLA-AMC substrate . The test system had a volume of 100 μL, with a final concentration of 0.5 μM for HsClpP protein and a final concentration of 200 μM for the AC-WLA-AMC substrate. The stock solution of preferred compounds were diluted into a series of gradients, with the final concentrations set at 10 μM, 1 μM, 500 nM, 250 nM, 125 nM, 62.5 nM, and 31.25 nM. The small molecules and the HsClpP protein solution were added to a flat-bottomed black 96-well plate, with three replicate wells for each group. After incubating at room temperature for 10 minutes, the AC-WLA-AMC substrate was added, and the fluorescence intensity in each well was immediately detected using a fluorescence plate reader (excitation wavelength: 360 nm, emission wavelength: 440 nm). Readings were taken every 5 minutes with 5 seconds of shaking before each reading, and continuous detection was performed for 30 minutes. DMSO was used as a negative control instead of small molecules. The multiple of readings at different concentrations against the readings of DMSO group was recorded as an index for evaluation of hydrolysis activity. . . . GraphPad Prism was used to plot the obtained multiple values and calculate the EC₅₀ values. The results in FIG. 2 showed that, as the concentration increases, the ability of ONC201 and the compound of the present invention to promote the hydrolysis of AC-WLA-AMC substrates by HsClpP protein increases in a concentration dependent manner.

TABLE 1
Regulatory efficacy*

| Cmpds | 10 μM | 1 μM |
|---|---|---|
| 1 | + + | + + |
| 2 | + | + |
| 3 | + | + |
| 4 | + + | + + |
| 5 | + + | + |
| 6 | + + | + + |
| 7 | + + | + + |
| 8 | + + | + + |
| 9 | + + | + |
| 10 | + + | + + |
| 11 | + + | + + |
| 12 | + + | + + |
| 13 | + + | + + |
| 14 | + + | + + |
| 15 | + + | + + |
| 16 | + + | + + |
| 17 | + + | + + |
| 18 | + + | + + |
| 19 | + + | + + |
| 20 | + + | + + |
| 21 | + + | + + |
| 22 | + + | + + |
| 23 | + + | + |
| 24 | + + | + |
| 25 | + + | + |
| 26 | + + | + + |
| 27 | + + | + + |
| 28 | + | + |
| 29 | + | + |
| 30 | + | + |
| 31 | + + | + |
| 32 | + + | + + |
| 33 | + + | + + |
| 34 | + + | + + |
| 35 | + + | + + |
| 36 | + + | + + |
| 37 | + + | + |
| 38 | + + | + + |
| 39 | + | + |
| 40 | + | + |
| 41 | + | + |
| 42 | + + | + |
| 43 | + + | + |
| 44 | + + | + |
| 45 | + | + |
| 46 | + + | + |
| 47 | + + | + + |
| 48 | + + | + + |
| 49 | + + | + + |
| 50 | + + | + + |
| 51 | + + | + + |
| 52 | + + | + + |
| 53 | + + | + + |
| 54 | + + | + + |
| 55 | + + | + + |
| 56 | + + | + + |
| 57 | + + | + + |
| 58 | + + | + + |
| 59 | + + | + + |
| 60 | + + | + + |
| 61 | + + | + + |
| 62 | + + | + + |
| 63 | + + | + |
| 64 | + + | + + |
| 65 | + + | + |
| 66 | + + | + + |
| 67 | + + | + + |
| 68 | + + | + + |
| 69 | + + | + + |
| 70 | + + | + |
| 71 | + + | + + |
| 72 | + + | + + |
| 73 | + + | + + |
| 74 | + + | + + |
| 75 | + + | + + |
| 76 | + + | + + |
| 77 | + + | + + |
| 78 | + + | + + |
| 79 | + + | + + |
| 80 | + + | + + |
| 81 | + + | + + |
| ONC201 | + + | + |

Table 1 shows the efficacy of the compounds of the present invention and the control compound ONC201 at concentrations of 10 μM and 1 μM in promoting the hydrolysis of short peptide substrate by HsClpP enzyme. * The efficacy relative to the blank control group: ++ indicating that the increased efficacy is greater than 50% of the blank control group; + indicating that the increased efficacy is less than 50% of the blank control group.

The results in Table 1 showed that the compound of the present invention and ONC201 demonstrated excellent regulatory performance against HsClpP at both 10 μM and 1 μM concentrations.

The EC₅₀ values for the excitatory activity of typical compounds on HsClpP are shown in Table 2:

TABLE 2

| Com- | | |
|---|---|---|
| pounds | Chemical structure | EC₅₀(μM) |
| 1 | 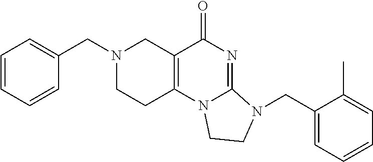 [SMILES: Cc1ccccc1CN1CCn2c1nc(=O)c1c2CCN(Cc2ccccc2)C1] | 0.30 |
| 15 | 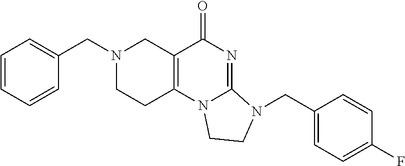 [SMILES: O=c1nc2n(c3c1CN(Cc1ccccc1)CC3)CCN2Cc1ccc(F)cc1] | 0.10 |
| 18 | 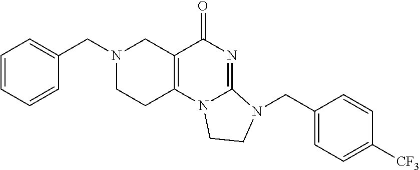 [SMILES: O=c1nc2n(c3c1CN(Cc1ccccc1)CC3)CCN2Cc1ccc(C(F)(F)F)cc1] | 0.30 |
| 21 | 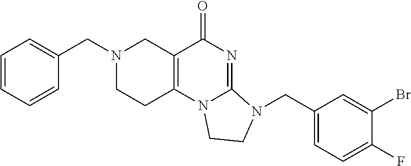 [SMILES: O=c1nc2n(c3c1CN(Cc1ccccc1)CC3)CCN2Cc1ccc(F)c(Br)c1] | 0.25 |
| ONC201 | 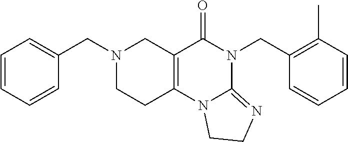 [SMILES: Cc1ccccc1CN1C(=O)C2=C(CCN(Cc3ccccc3)C2)N2CCN=C12] | 1.00 |

Table 2: The EC₅₀ values for the excitatory activity of the compounds of the present invention and the control compound ONC201 on HsClpP.

By comparing Compound 1 of the present invention with the compound ONC201, which has entered clinical trial stages, it can be seen that the two compounds differ only in the connecting position of the N atom between 2-methylphenyl and the tricyclic ring. However, the present inventors unexpectedly found that the EC₅₀ of Compound 1 against HsClpP is 2.3 times lower than that of ONC201, suggesting that the compounds of the present invention exhibit superior therapeutic effects against HsClpP-mediated diseases.

Example 3: In vitro anti-tumor proliferation assay of the compounds. The purpose of this experiment was to use CCK-8 to detect the in vitro inhibitory activity of the compounds of the present invention against the proliferation of tumor cells. The main reagents, such as RPMI-1640, DMEM high-glucose culture medium, fetal bovine serum, trypsin, etc., were purchased from Gibco BRL. CCK8 and DMSO were products of Sigma. For in vitro experiment, the test compounds were prepared as 10 mM stock solutions in DMSO and stored at −20° C. in the refrigerator in the dark for later use, then diluted to desired concentration with complete culture medium before use. The human lung cancer cells, colon cancer cells, breast cancer cells, glioblastoma cells, human myelomonocytic leukemia cells, and human Burkitt's lymphoma cells used in this experiment were all purchased from the American ATCC company and preserved in laboratory of the inventors. All the above cell lines were cultured in RPMI-1640 complete medium or DMEM complete medium containing 10% fetal bovine serum, 100 U/mL penicillin, and 100 μg/mL streptomycin under 5% carbon dioxide, 37° C. conditions. Experimental method: When the cells were observed to be in good growth during the cell culture process, the cells were digested and collected by centrifugation. The previous culture medium was discarded, fresh culture medium was added to resuspend the cells, and then the cells were counted. According to the growth rate of different cells, the cell seeding concentration was determined, generally being 3000-5000 cells/well. After determining the seeding concentration, the cell suspension was diluted to desired concentrations with fresh culture medium, and then added to a 96-well plate with 100 μL per well, and 200 μL of PBS was added to the edge wells to prevent evaporation of the culture medium. On the second day, drug treatment was performed. First, the compounds were diluted into a series of gradients with culture medium, and then the drug solution was added to the 96-well plate, with 3 parallel wells for each gradient. Each plate set a blank control group, and ONC201 was used as the positive control group. After 72 hours of drug treatment, the growth status of cells in the 96-well plate was observed with naked eyes, and then the CCK-8 solution was added to each well and then the 96-well plate was incubated in the incubator for 1-2 hours before measuring the absorbance at 450 nm. The inhibition rate at each drug concentration was calculated, and the cell inhibition rate=(OD450 of the blank control group-OD450 of the experimental group)/OD450 of the blank control group ×100%. Then, Graphpad prism software was used for ICso calculation, and the results were shown in Tables 3, 4, and 5.

TABLE 3

| | IC₅₀(uM) | | | | | |
|---|---|---|---|---|---|---|
| Cmpds | SW620 | DLD1 | A549 | MV-4-11 | Raji | A172 |
| 1 | 0.6 | 0.2 | 2 | 0.8 | 1 | 0.5 |
| 18 | 0.5 | 0.15 | 0.2 | 0.05 | 0.1 | 0.2 |
| 19 | 0.15 | 0.2 | 0.4 | 0.05 | 0.1 | 0.15 |
| 20 | 0.14 | 0.5 | 0.5 | 0.025 | 0.1 | 0.1 |
| 21 | 0.06 | 0.025 | 0.05 | 0.006 | 0.025 | 0.03 |
| ONC201 | 9 | 2.5 | 10 | 5 | 2.5 | 2.5 |

Table 3 shows the inhibitory activity of preferred compounds 1, 18-21, and ONC201 against 5 colon cancer cells (SW620, DLD-1), human non-small cell lung cancer cells (A549), human myelomonocytic leukemia cells (MV-4-11), human Burkitt's lymphoma cells (Raji), and human glioblastoma cells (A172).

TABLE 4

| Com- | IC₅₀ ( M) | | | | | |
|---|---|---|---|---|---|---|
| pound | HCT116 | HCT115 | SW620 | HT29 | DLD1 | SW480 |
| 1 | 0.4 | 1.3 | 0.6 | 5.3 | 0.5 | 1.1 |
| 15 | 0.08 | 0.2 | 0.19 | 0.4 | 0.09 | 0.17 |
| 16 | 0.08 | 0.25 | 0.17 | 0.07 | 0.1 | 0.1 |
| 19 | 0.09 | 0.12 | 0.09 | 0.08 | 0.09 | 0.12 |
| 20 | 0.1 | 0.2 | 0.15 | 0.05 | 0.15 | 0.12 |
| 21 | 0.04 | 0.1 | 0.06 | 0.05 | 0.08 | 0.07 |
| 1 | 4.0 | 10.2 | 9.2 | 35.0 | 5.5 | 10 |
| ONC201 | | | | | | |

Table 4 shows the inhibitory activity of preferred compounds 1, 15-16, 19-21, and ONC201 against the proliferation of human colorectal cancer cells (SW620, HCT116, HCT115, HT29,

SW480, DLD1) in vitro.

TABLE 5

| | IC₅₀ | | IC₅₀ | | IC₅₀ | | IC₅₀ |
|---|---|---|---|---|---|---|---|
| Cmpds | (M) | Cmpds | (M) | Cmpds | (M) | Cmpds | (M) |
| 1 | 0.4 | 11 | 0.3 | 17 | 0.5 | 26 | 0.03 |
| 2 | >10 | 12 | 0.2 | 18 | 0.3 | 28 | >10 |
| 5 | >10 | 13 | 1.3 | 19 | 0.1 | 32 | 1.5 |
| 6 | 2.5 | 14 | 1.2 | 20 | 0.1 | 35 | 0.5 |
| 7 | 0.5 | 15 | 0.08 | 21 | 0.04 | 39 | >10 |
| 8 | 0.3 | 16 | 0.08 | 22 | 0.08 | ONC201 | 4 |

Table 5 shows the inhibitory activity of the compounds of the present invention and ONC201 against the proliferation of human colorectal cancer cells (HCT116) in vitro.

In Table 3, compared to the positive control ONC201, the inhibitory activity of the preferred compounds against colorectal cancer cells (SW620, DLD-1), human non-small cell lung cancer cells (A549), human myelomonocytic leukemia cells (MV-4-11), human Burkitt's lymphoma cells (Raji), and human glioblastoma cells (A172) significantly increased. Some preferred compounds exhibited one to three orders of magnitude more active, demonstrating great potential in the development of anti-cancer drugs. In Table 4, the preferred compounds showed significantly better in vitro inhibitory activity against colorectal cancer-related tumor cells compared to the positive control ONC201, highlighting the importance of the invention in the field of colorectal cancer treatment. In Table 5, most compounds exhibited excellent anti-proliferative effects against HCT116 cells, with some compounds showing 1-100 times higher anti-proliferative activity than the positive control compound.

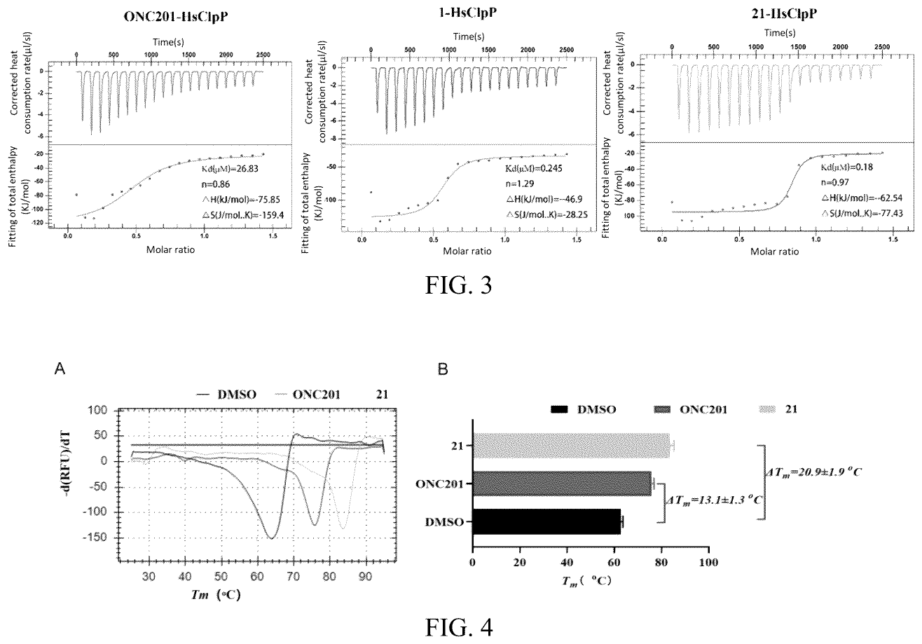

Example 4: Isothermal Titration calorimetry (ITC) Experiment. Isothermal Titration calorimetry experiments can measure the binding affinity between small molecules and proteins. Binding affinity is generally measured and reported through the equilibrium dissociation constant (Kd), where the smaller the Kd value, the higher the binding affinity of the ligand for its target. In the experiment, the loading-samples and proteins were placed in a degassing apparatus at 30° C., with a vacuum degree of 400 mmHg or higher, and degassed for 10 minutes. A 50 mL PBS buffer was prepared, and the sample cell and syringe were rinsed. After rinsing, the liquid in the sample cell was completely removed, and the degassed protein and small molecules were taken out. The sample cell was thoroughly washed with protein, and the syringe was rinsed with the small molecule solution 3-5 times. After washing, 250 μL of protein (0.5 mM) was extracted using the syringe, avoiding the formation of bubbles. Subsequently, 200 μL was injected into the sample cell. 50 μL of the small molecule solution (0.1 mM) was extracted using an injector, again avoiding the formation of bubbles. The titration syringe was loaded onto the instrument, and titration commenced after the instrument was balanced. As shown in FIG. 3, the results of the ITC experiment indicated that the titration of ONC201, 1, and 21 with HsClpP showed AH<0, AS<0, suggesting a specific hydrogen bond formation between the compounds and HsClpP. Moreover, the Kd values indicated that compounds 1 and 21 have a much higher affinity for HsClpP compared to ONC201, with an affinity level increase by up to 2 orders of magnitude.

Example 5: Differential Scanning Fluorimetry (DSF) Experiment. This experiment verified the interaction between compouds of the invention and HsClpP by assessing the impact of ONC201 and the preferred compound 21 on the stability of the HsClpP protein at the same concentration, and compared the strength of the interaction between thecompounds of the invention and ONC201 with HsClpP. 10 μM HsClpP, 5×SSYPRO Orange, and 100 μM compound solutions were separately added to RT-PCR eight-tube strips, with at least 2 duplicate wells for each condition and then incubated at room temperature for 30 min. The reaction system consisted of K₂HPO₄/KH₂PO₄ 50 mM pH 7.6, KCl 100 mM, and 5% glycerol. Detection was carried out using an RT-PCR instrument for fluorescence detection. The melting curve was selected, with the temperature setting to rise from 25° C. to 99° C. within 40 minutes. Fluorescence detection was performed using the SSYPRO Orange channel, and the data were recorded. As shown in FIG. 4, using DMSO as a blank control, the above compounds caused a right shift in the Tm value of the HsClpP protein, indicating a significant impact on the thermal stability of HsClpP. At the same concentration of 100 μM, the change in Tm value caused by compound 21 was greater than that of ONC201, suggesting that compound 21 of the present invention had a superior effect on the thermodynamic stability of the HsClpP protein compared to ONC201, making it an excellent HsClpP regulator.

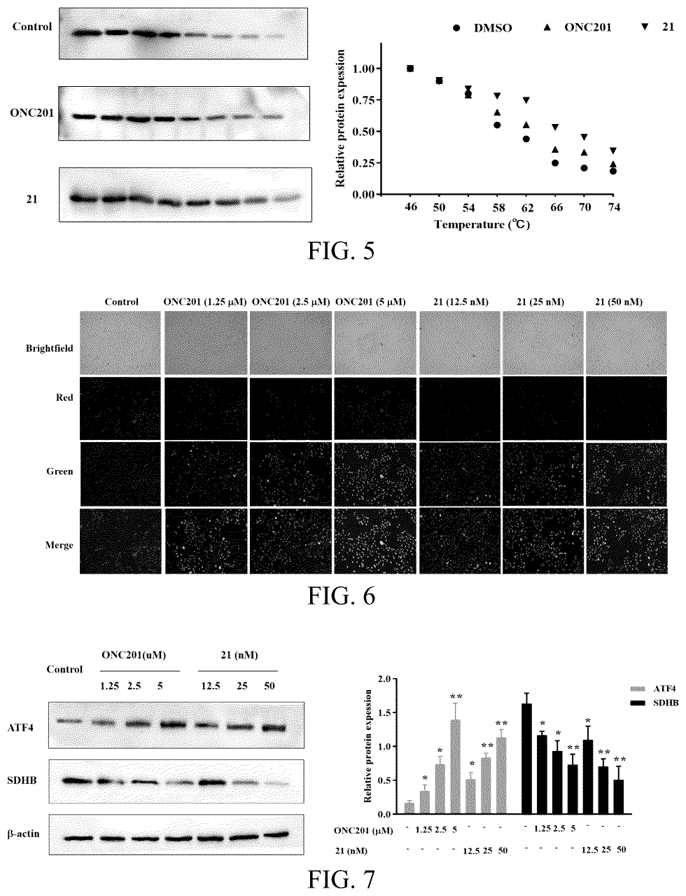

Example 6: Cellular Thermal Shift Assay (CETSA). The purpose of this experiment is to investigate the targeting effect of the preferreed compounds on HsClpP in a cellular environment. The general procedure is as follows: cells are treated with the compound, heated to induce protein denaturation and precipitation, and soluble proteins are separated from cell fragments and precipitates for detection. Proteins not binding to the ligand undergo denaturation and precipitation at high temperatures, while ligand-bound proteins remain in the solution. Quantitative western blotting is then used to analyze the samples. The specific steps are as follows: HCT116 cells in logarithmic growth phase are incubated with 100 μM of compound for 30 minutes, and the cells are collected in a 15 mL BD tube for centrifugation at 2000 rpm for 5 minutes to remove the supernatant, washed twice with 1 mL pre-cooled PBS, transferred to 1.5 mL EP tubes, and centrifuged at 5000 rpm for 10 minutes. Cell lysis buffer RIPA is added, and the cells are ice-bathed for at least 30 minutes. The cells are then broken by ultrasonication until the solution is colorless and transparent, and then centrifuged at 4° C., 13300 rpm, for 10 minutes. The supernatant is equally-divided into 8 portions, with temperature gradients of 46, 50, 54, 58, 62, 66, 70, and 74° C., respectively and each of the eight samples corresponds to one temperature. each samples is heated for 3 minutes using a PCR instrument, and then quickly placed on ice to chill down. The samples are then centrifuged at 4° C., 13300 rpm, for 10 minutes. Western blotting is used to detect changes in the HsClpP protein content in the experimental and control groups. As shown in FIG. 5, when the cell treated with 100 μM of compound 21 was subsequently split, the soluble portion was determined by western blot assay. The results indicate that compound 21 can effectively stabilize the HsClpP protein at different temperatures. In contrast, ONC201 is not as good as compound 21 in maintaining HsClpP stability at the same concentration, suggesting that the preferred compound 21 can enter HCT116 cells, target HsClpP, and enhance its thermal stability. The results of the CETSA experiment further validate the correlation between the anti-tumor activity of the compound and its targeting of HsClpP.

Example 7: Induction of Mitochondrial Membrane Potential by preferred Compound 21. In this study, the membrane potential changes are tested using the tetramethylrhodamine methyl ester (TMRM) probe. As shown in FIG. 6, compared to the positive compound ONC201, the ability of the preferred compound 21 to induce changes in mitochondrial membrane potential is more pronounced and concentration-dependent.

Example 8: Influence of Preferred Compound 21 on the Levels of Mitochondrial Respiratory Chain Complexes and Endoplasmic Reticulum Oxidative Stress Response. Literature studies have indicated that dysfunction of HsClpP reduces the enzymatic activity of Complex II in the cell, with a faster migration rate of the SDHB protein. This may suggest the accumulation of non-functional misfolded SDHB or degradation of SDHB, indicating the importance of SDHB as a substrate for HsClpP function and the role of HsClpP in maintaining oxidative phosphorylation of cellular subpopulation. ATF proteins, as common markers for endoplasmic reticulum oxidative stress, play a crucial role in monitoring endoplasmic reticulum stress. Disruption of HsClpP function activates the unfolded protein response, thereby influencing endoplasmic reticulum stress. In this experiment, at the level of HCT116 cells, the impact of the preferred compound on the levels of SDHB and ATF proteins was evaluated. As shown in FIG. 7, HCT116 cells treated with compound 21 and ONC201 can both effectively and dose-dependently lead to ATF4 accumulation and a reduction in SDHB level. This suggests that the compromised mitochondrial proteolytic function induced by compound 21 may impair mitochondrial respiratory function and oxidative phosphorylation levels.

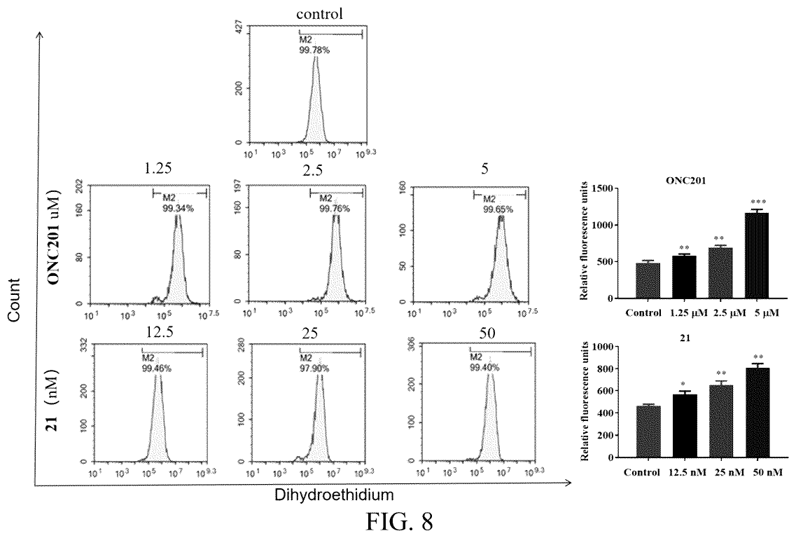

Example 9: Induction of ROS generation by Compound 21. In cancer cells, mitochondrial reactive oxygen species (ROS) are closely associated with tumorigenesis. Disruption of HsClpP function increases the generation of mitochondrial ROS, thereby inducing apoptosis in tumor cells. In this experiment, HCT116 cells were treated with compound 21 at different concentrations , incubated continuously for 48 hours, and then stained with DCFH-DA. Changes in intracellular ROS content were observed under a fluorescence microscope. As shown in FIG. 8, the changes in intracellular ROS content was concentration-dependent with drug, indicating that this compound can induce the generation of ROS.

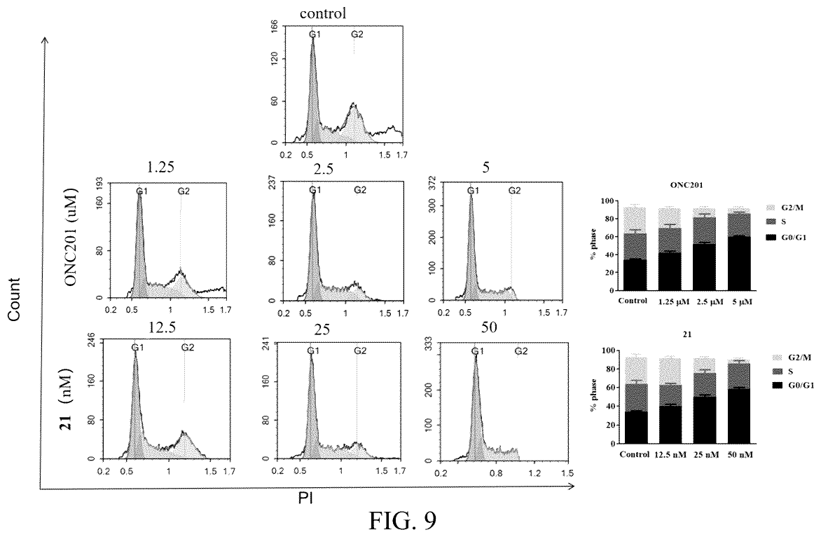

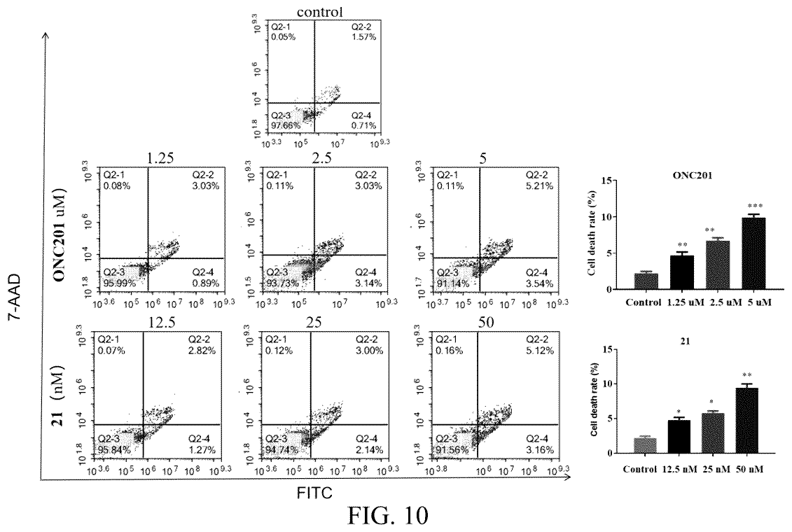

Example 10: Induction of Tumor Cell Cycle Arrest and Apoptosis by Preferred Compound 21. This study employed flow cytometry to investigate the impact of compound 21 at doses of 12.5, 25, and 50 nM on cell cycle arrest and apoptosis in HCT116 cells. As shown in FIG. 9, the control group had a GO/G1 phase cell count of 26%, while 12.5 nM of compound 21 increased the percentage of GO/G1 phase cells to 40%. As the concentration increased, the number of GO/G1 phase cells increased linearly. At a dose of 50 nM, the number of GO/G1 phase cells ultimately reached 55%, indicating that compound 21 can significantly induce GO/G1 phase cell arrest in a concentration dependent manner. When the concentration was 100 times higher than compound 21, ONC201 exhibited a similar effect. It was speculated that compound 21 may promote cancer cell apoptosis by inducing cell cycle arrest. Annexin V/PI staining was used to detect the apoptotic effect induced by compound 21. As shown in FIG. 10, both compound 21 and ONC201 induced apoptosis in a concentration-dependent manner. The apoptotic rate induced by compound 21 at a concentration of 12.5 nM was 4.09%, which was higher than that induced by ONC201 at a concentration of 1.25 μM. Overall, the apoptotic rate induced by compound 21 increased linearly with increasing concentration.

Example 11: In vitro Antitumor Cell Colony Formation Assay of preferred Compound. In addition to being closely associated with the proliferation of tumor cells, the regulation of HsClpP also induces cell cycle arrest and the formation of clones. In this experiment, HCT116 cells were used for a cell monoclonal experiment, and stained with crystal violet. As shown in FIG. 11, the preferred compound 21 at concentrations of 12.5nM, 25nM, and 50nM significantly inhibited the formation of HCT116 mono-clones.

Example 12: In vitro Antitumor Cell Migration Assay of Preferred Compound. Studies have shown that increased expression of HsClpP is crucial for the proliferation and migration of certain cancer cell lines, while functional disruption of HsClpP in cancer cells can inhibit cell migration.

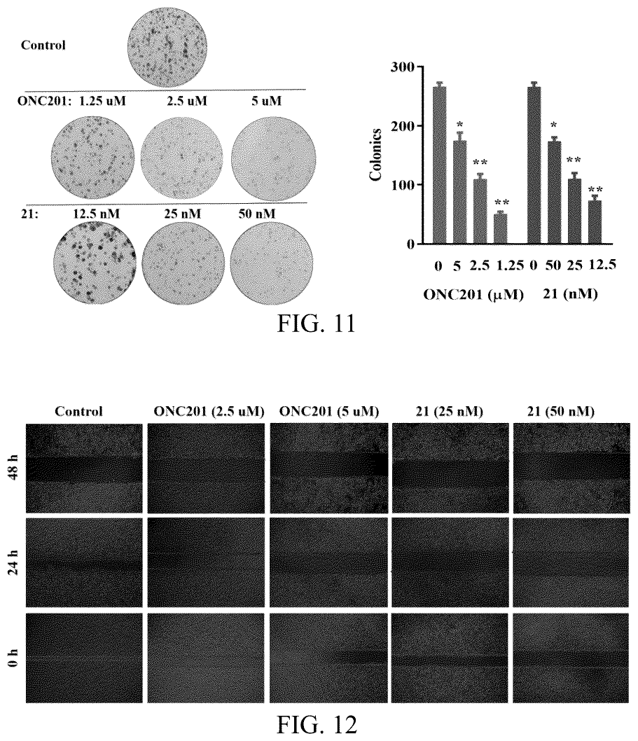

Wound-Healingcan be used to detect the invasive ability of adherent tumor cells. By observing the healing ability of tumor cells towards scratches under different conditions, the impact of the compound on the ability of tumor cell migration can be reflected. As shown in FIG. 12, in this study, after incubating the preferred compound 21 with HCT116 cells for 24 hours, the migration ability of HCT116 cells was significantly inhibited compared to the control group and the ONC201 group.

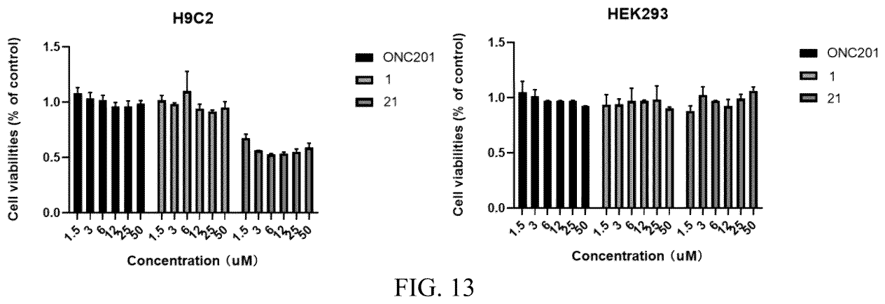

Example 13: In vitro Cytotoxicity Assay of Preferred Compound. The purpose of this experiment was to use CCK-8 to detect the inhibitory activity of preferred compounds on the proliferation of human normal embryonic kidney cells (HEK293) and rat myocardial cells (H9C2) in vitro. The cell culture method, administration method, and detection method were the same as in example 9. As shown in FIG. 13, for HEK293 cells, compounds 1 and 21 showed no significant cytotoxicity, similar to the control ONC201, even at a concentration of up to 50μM. For H9C2 cells, compounds 1, 21, and ONC201 showed no significant inhibitory activity.

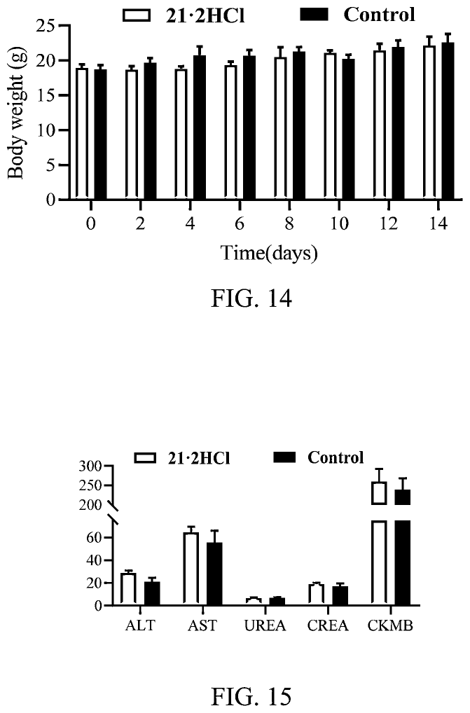

Example 14: Acute In vivo Toxicity of Preferred Compounds in animals. This experiment aimed to evaluate the safety of the hydrochloride salt of the preferred compound 21 by single-dose acute toxicity using male and female BALB/c mice. No adverse reactions were observed after a single gavage dose of 100 mg/kg. Within two weeks after administration of the hydrochloride salt of compound 21, no significant weight loss was observed (FIG. 14). There was no significant difference in blood biochemistry compared to the control group ONC201 hydrochloride salt (FIG.

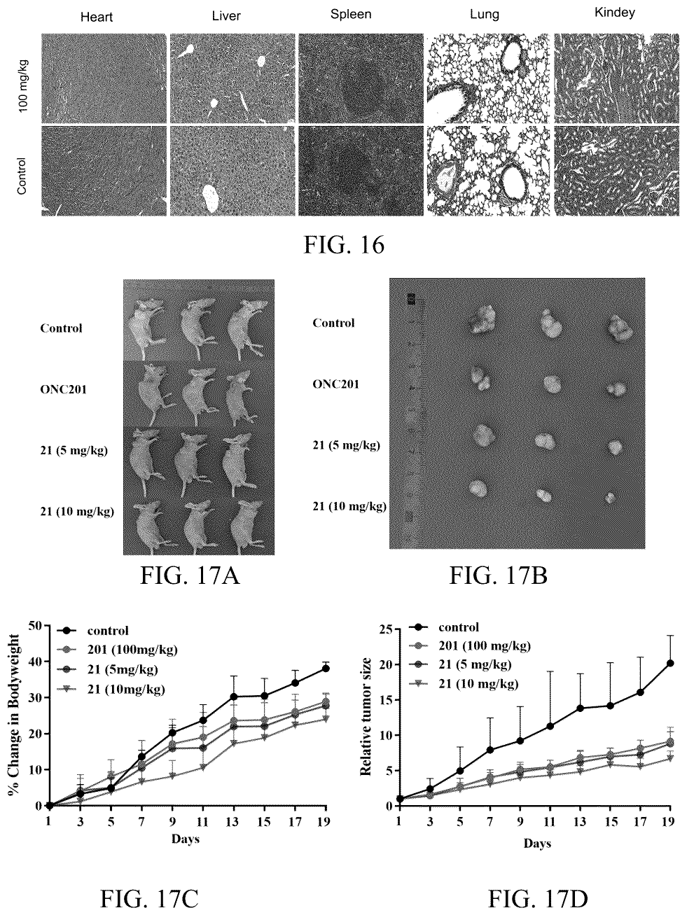

15). In addition, HE staining analysis showed no apparent pathological damage at a dose of 100 mg/kg (FIG. 16).

Example 15: In vivo Antitumor Activity of Preferred Compound in animals. This experiment evaluated the antitumor effects of ONC201 hydrochloride salt and the hydrochloride salt of preferred compound 21 in a xenograft nude mouse model inoculated with HCT116 cells. As shown in FIG. 17, mice were orally administered with 100 mg/kg ONC201 hydrochloride salt twice a week and concurrently received the hydrochloride salt of 21 at doses of 5 mg/kg and 10 mg/kg. After a total of six oral (gavage) doses, oral administration of 100 mg/kg ONC201 hydrochloride salt group showed good inhibition of antitumour activity, with a TGI (tumor growth inhibition) of 57%. In the 21 hydrochloride salt group, tumor growth inhibition was observed, with the corresponding inhibition rates for the 5 mg/kg and 10 mg/kg dose groups being 56% and 67%, respectively.

Those skilled in this field should understand that changes can be made to the exemplary embodiments shown and described above without departing from the broad inventive concept. The present invention is not limited to the exemplary embodiments shown and described but rather covers the spirit and scope of the present invention as defined by the claims.

1. A compound as showed in formula I, : 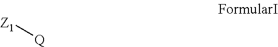
wherein Z₁ is independently selected from H, alkyl, cycloalkyl, cycloalkylalkyl, heterocycloalkyl, heterocycloalkylalkyl, aryl, heteroaryl, arylalkyl, heteroarylalkyl, alkyloxyalkyl, alkoxycarbonyl, arylalkoxy, arylalkylthio, and acyl radical; Q is independently selected from the group consisting of: 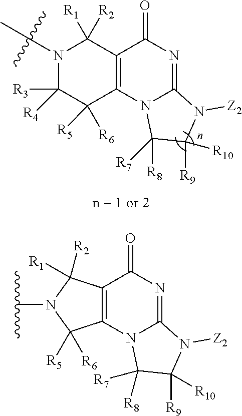
in each formula, R₁ to R₆ are each independently selected from hydrogen, halogen, C3-C6 cycloalkyl, and C1-C6 unsubstituted or substituted alkyl; each of R₇ to R₁₀ is independently selected from hydrogen, halogen, C3-C6 cycloalkyl, and C1-C6 unsubstituted or substituted alkyl; Z₂ is independently selected from alkyl, cycloalkyl, cycloalkylalkyl, heterocycloalkyl, heterocycloalkylalkyl, aryl, heteroaryl, arylalkyl, heteroarylalkyl, alkoxyalkyl, alkoxycarbonyl, arylalkoxy, arylalkylthio, and acyl; and n=1 or 2.

2. The compound according to claim 1, wherein the compound is represented by Formula I-1: 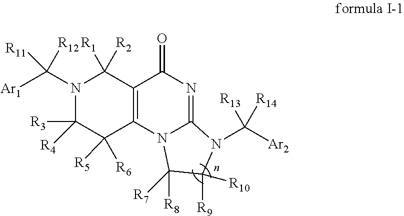
wherein, Ar₁ and Ar₂ are independently selected from aryl, heteroaryl, alkyl, alkenyl, alkynyl, arylalkyl, cycloalkyl, cycloalkylalkyl, and heterocycloalkyl; said aryl, heteroaryl, alkyl, alkenyl, alkynyl, arylalkyl, cycloalkyl, cycloalkylalkyl, or heterocycloalkyl independently have 0-5 (such as 1, 2, 3, or 4) R₁₅ substituents; each R₁₅ is independently selected from halogen, cyano, C1-C6 alkyl, C3-C9 unsubstituted or substituted cycloalkyl, C1-C6 haloalkyl, —CF₃, —NH₂, —NO₂, —SH, —SR₁₆, —OH, C1-C6 unsubstituted or substituted alkoxy, —NR₁₆R₁₇, (C3-C9) cycloalkyl, (C2-C6) alkynyl, (C4-C8) cycloalkenyl, (C4-C8) cycloalkenylalkyl, unsubstituted or substituted aryl, unsubstituted or substituted heteroaryl, —COOH, —COOR₁₆, —OCOOR₁₆, C2-C8 alkenyl, —SO₂OR₁₆, —SO₂NR₁₆R₁₇, —SO₂R₁₆, —NR₁₆SO₂R₁₇, —CONR₁₆R₁₇, —COR₁₆, —NR₁₆COR₁₇; R₁ to R₁₀ are each independently selected from hydrogen, halogen, C3-C6 cycloalkyl, and C1-C6 unsubstituted or substituted alkyl;
each of R₁ to R₁₇ is independently selected from hydrogen, halogen, C1-C3 unsubstituted or substituted alkyl, or R₁₁ and R₁₂ together with the connected C atom form a carbonyl group (C═O);
and n=1 or 2.

3. The compound according to claim 2, wherein Ar₁ and Ar₂ are independently selected from the group consisting of: phenyl, naphthyl, quinolinyl, indolyl, benzofuranyl, pyridyl, thiadiazolyl, thiazolyl, C1-C6 alkyl, C2-C6 alkenyl, C2-C6 alkynyl, C3-C8 cycloalkyl, C3-C8 cycloalkyl-C1-C3 alkylene, phenyl-C1-C3 alkylene, thienyl, and furanyl; preferably, the phenyl, naphthyl, quinolinyl, indolyl, benzofuranyl, pyridyl, thiadiazolyl, thiazolyl, C1-C6 alkyl, C2-C6 alkenyl, C2-C6 alkynyl, C3-C8 cycloalkyl, C3-C8 cycloalkyl-C1-C3 alkylene, phenyl-C1-C3 alkylene, thienyl, and furanyl are optionally and independently substituted with 0-5 R₁₅ substituents, and each R₁₅ is independently selected from the group consisting of: halogen, —OH, cyano, C1-C6 alkyl, C1-C6 alkoxy, C1-C6 haloalkyl, —SO₂C1-C3 alkyl, COOC1-C6 alkyl, and COOH.

4. The compound according to claim 2, wherein, Ar₁ is selected from the group consisting of:
phenyl, phenyl-C1-C3 alkylene, 4-cyanophenyl, 3-cyanophenyl, 2-cyanophenyl, 3-fluorophenyl, 4-fluorophenyl, 2-fluorophenyl, 3,4-difluorophenyl, 2, 4-difluorophenyl, pyridin-2-yl, pyridin-3-yl, pyridin-4-yl, 2-methylphenyl, 3-methylphenyl, 4-methylphenyl, 2-methoxyphenyl, 3-methoxyphenyl, 4-methoxyphenyl, cyclopropyl, cyclobutyl, cyclopentyl, cyclohexyl, furan-3-yl, ethynyl, and C1-C6 alkyl.

5. The compound according to claim 2, wherein, Ar₂ is selected from the group consisting of:
phenyl, 2-methoxyphenyl, 3-methoxyphenyl, 4-methoxyphenyl, 2-methylphenyl, 3-methylphenyl, 4-methylphenyl, 2-trifluoromethylphenyl, 3-trifluoromethylphenyl, 4-trifluoromethylphenyl, 2-fluorophenyl, 2-chlorophenyl, 2-bromophenyl, 3-fluorophenyl, 3-chlorophenyl, 3-bromophenyl, 4-fluorophenyl, 4-chlorophenyl, 4-bromophenyl, 3,4-difluorophenyl, 3,4-dichlorophenyl, 3,4-dibromophenyl, 2, 4-difluorophenyl, 2, 4-dichlorophenyl, 2, 4-dibromophenyl, 2-bromo-3-fluorophenyl, 3-bromo-4-fluorophenyl, 2-chloro-4-fluorophenyl, 4—CH₃SO₂-phenyl, cyclopropyl, cyclobutyl, cyclopentyl, ethynyl, cyclohexyl, C1-C6 alkyl, phenyl-methylene, pyridin-2-yl, pyridin-3-yl, pyridin-4-yl, and 3—CH₃COO-phenyl.

6. The compound according to claim 2, wherein, the compound is shown in Formula I-2: 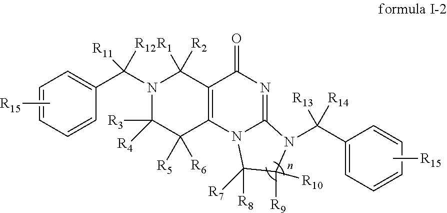
wherein, Ar₁ and Ar₂ are phenyl, each phenyl independently have 0-5 R₁₅ substituents, and each R₁₅ is independently selected from the group consisting of: halogen, cyano, C1-C6 alkyl, C3-C9 substituted or unsubstituted cycloalkyl, C1-C6 halogenated alkyl, —CF₃, —NH₂, —NO₂, —SH, —SR₁₆, —OH, C1-C6 substituted or unsubstituted alkoxy, —NR₁₆R₁₇, (C3-C9) cycloalkyl, (C2-C6) alkynyl, (C4-C8) cycloalkenyl, (C4-C8) cycloalkenylalkyl, substituted or unsubstituted aryl, substituted or unsubstituted heteroaryl, —COOH, —COOR₁₆, —OCOOR₁₆, C2-C6 alkynyl, C2-C8 alkenyl, —SO₂OR₁₆, —SO₂NR₁₆R₁₇, —SO₂R₁₆, —NR₁₆SO₂R₁₇, —CONR₁₆R₁₇), —COR₁₆, —NR₁₆COR_{17;}
R₁ to R₆, R₇ to R₁₀ are each independently selected from hydrogen, halogen, C3-C6 cycloalkyl, and C1-C6 substituted or unsubstituted alkyl;
R₁₁ to R₁₇ are each independently selected from hydrogen, halogen, and C1-C3 substituted or unsubstituted alkyl;
and n=1 or 2.

7. The compound according to claim 6, wherein R₁ to R₁₄ are independently selected from hydrogen, halogen, and C1-C3 substituted or unsubstituted alkyl; R₁₅ is selected from hydrogen, halogen, cyano, —CH₃, and—CF_{3.}

8. The compound according to claim 1, wherein, the compound is selected from: 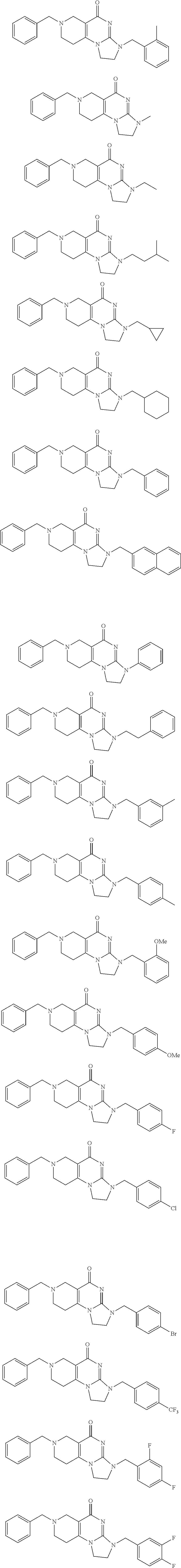 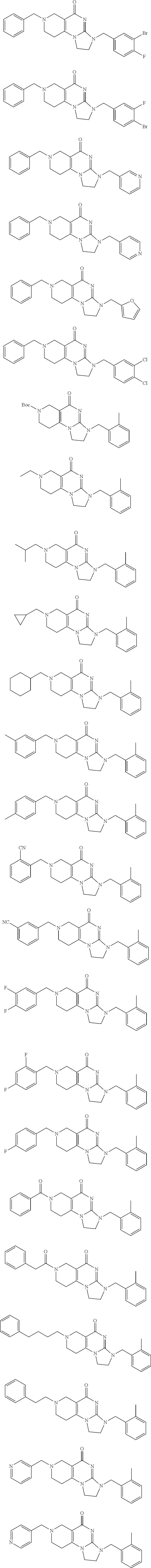 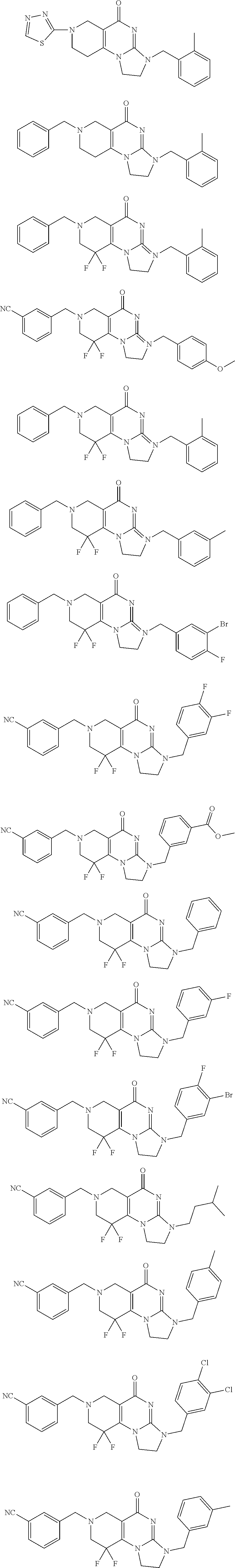 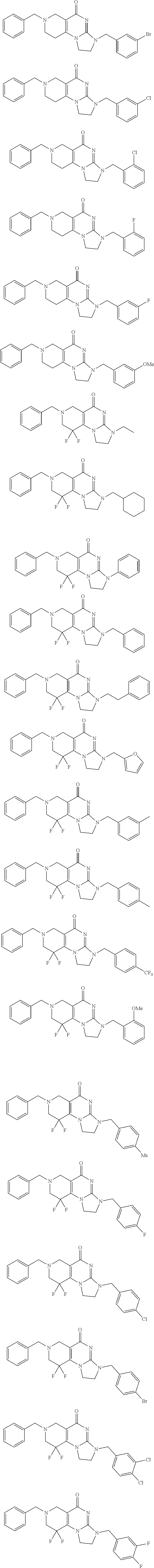 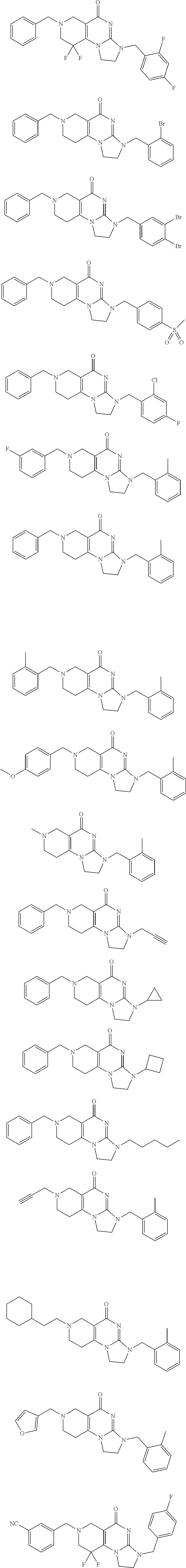 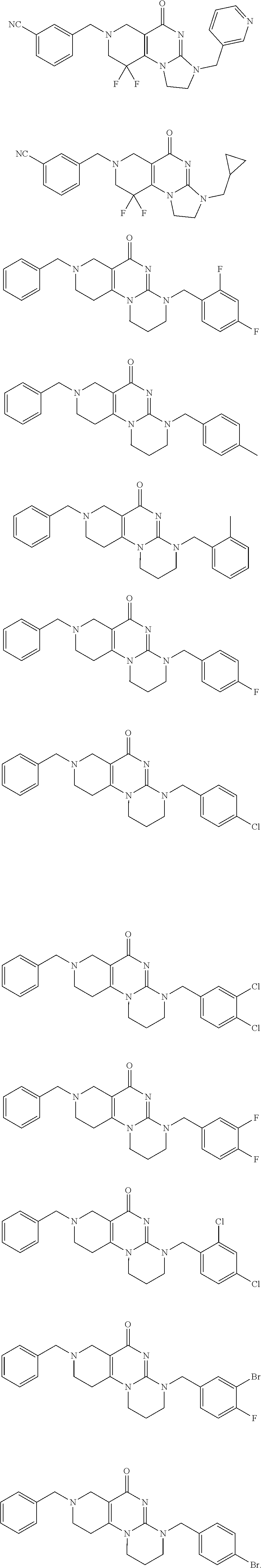
Docket No .: PBA408.0140 Docket No .: PBA408.0140

9. The compound according to claim 1, wherein any one or more atoms of the compound are replaced by their isotopes, preferably deuterium.

10. The compound according to claim 1, further comprising pharmaceutically acceptable salts, hydrates, solvates, or crystal forms of the compound; preferably, the pharmaceutically acceptable salts are pharmaceutically acceptable salts formed by the compound in combination with hydrochloric acid, hydrobromic acid, hydrofluoric acid, sulfuric acid, phosphoric acid, nitric acid, formic acid, acetic acid, propionic acid, oxalic acid, malonic acid, succinic acid, fumaric acid, maleic acid, lactic acid, malic acid, tartaric acid, citric acid, picric acid, methanesulfonic acid, ethanesulfonic acid, hydroxyethanesulfonic acid, p-toluenesulfonic acid, benzenesulfonic acid, naphthalenesulfonic acid, trifluoroacetic acid, or aspartic acid.

11. A pharmaceutical composition, comprising the compound of claim 1 or a pharmaceutically acceptable salt, a hydrate, a solvate, or a crystal form thereof as an active ingredient, and pharmaceutically acceptable excipients.

12. A use of the compound of claim 1, or a pharmaceutically acceptable salt, a hydrate, a solvate, a crystal form thereof, or the pharmaceutical composition containing it, in the preparation of a drug for the prevention and/or treatment of HsClpP-mediated neurological diseases, metabolic syndrome, and tumor-related diseases.

13. A use of the compound of claim 1, or a pharmaceutically acceptable salt, a hydrate, a solvate, a crystal form thereof, or the pharmaceutical composition containing it, in the preparation of a drug for the prevention and/or treatment of tumors, preferably, the tumors are selected from the group consisting of: central nervous system tumors, brain tumors, peripheral nervous system tumors, chromaffin cell tumors, paraganglioma, neuroendocrine tumors, liver cancer, lung cancer, gastric cancer, colon cancer, rectal cancer, pancreatic cancer, breast cancer, prostate cancer, endometrial cancer, hematological malignancies, lymphocytic malignancy, gliomas, myeloid mononuclear leukemia, Burkitt's lymphoma, non-small cell lung cancer, glioblastoma, colorectal cancer, melanoma, ovarian cancer, or combinations thereof.
